(19) United States
(12) Patent Application Publication
(10) Pub. No.: US 2014/0037649 A1
(43) Pub. Date: Feb. 6, 2014

(54) DIAGNOSTIC AND/OR SCREENING AGENTS AND USES THEREFOR

(71) Applicants: Richard Bruce Brandon, Boonah (AU); Mervyn Rees Thomas, Chapel Hill (AU); Glenn Stone, Glenwood (AU)

(72) Inventors: Richard Bruce Brandon, Boonah (AU); Mervyn Rees Thomas, Chapel Hill (AU); Glenn Stone, Glenwood (AU)

(73) Assignee: IMMUNEXPRESS PTY LTD, TOOWONG (AU)

(21) Appl. No.: 13/989,738

(22) PCT Filed: Nov. 24, 2011
(86) PCT No.: PCT/AU11/01540

§ 371 Date: Oct. 17, 2013

Related U.S. Application Data
(60) Provisional application No. 61/417,381, filed on Nov. 26, 2010.

(51) Int. Cl. C12Q 1/68

(52) U.S. Cl. CPC C12Q 1/6876
USPC 424/164.1; 506/9; 435/6.12; 514/1.4

(57) ABSTRACT

Disclosed are methods and apparatus for diagnosis, detection of host response, monitoring, treatment or management of sepsis, infection-negative systemic inflammatory response syndrome (SIRS) and post-surgical inflammation in mammals. More particularly, the present invention discloses marker genes and their splice variant transcripts as well as their expression products, which are useful for distinguishing between sepsis and infection-negative SIRS, including post-surgical inflammation, and to the use of these markers in grading, monitoring, treatment and management of these conditions.

### FIELD OF THE INVENTION

This invention relates generally to methods and apparatus for diagnosis, detection of host response, monitoring, treatment or management of sepsis, infection-negative systemic inflammatory response syndrome (SIRS) and post-surgical inflammation in mammals. More particularly, the present invention relates to marker genes and their splice variant transcripts as well as their expression products that are useful for distinguishing between sepsis and infection-negative SIRS, including post-surgical inflammation, and to the use of these markers in grading, monitoring, treatment and management of these conditions. The invention has practical use in early diagnosis, diagnosis of mild or sub-clinical sepsis or infection-negative SIRS or post-surgical inflammation, in the detection of specific cell immune responses as part of active or progressive disease, in monitoring clinically affected subjects, and in enabling better treatment and management decisions to be made in clinically and sub-clinically affected subjects. Additionally, the invention has practical use in monitoring and grading patients in critical care or intensive care units for sepsis or infection-negative SIRS or post-surgical inflammation, and in predicting clinical outcome.

### BACKGROUND OF THE INVENTION

Systemic Inflammatory Response Syndrome (SIRS) is characterized by fever or hypothermia, leukocytosis or leukopenia, tachypnea and tachycardia. SIRS may have an infectious or non-infectious etiology and is described in association with critical conditions that include pancreatitis, ischemia, multi-trauma and severe tissue injury. Major open surgery is a controlled form of physical insult that results in varying degrees of systemic inflammation. In fact, it has been reported that the occurrence of SIRS following cardiac bypass surgery (Chello et al., 2006, Critical Care Medicine 34(3):660-667), open abdominal aortic repair (Brown et al., 2003, Journal of Vascular Surgery 37(3):600-606) and open colorectal resection (Haga et al., 1997, Critical Care Medicine 25(12):1994-2000) is very common, as well as a major cause of postoperative complications including death. Published findings by Michalopoulos and colleagues indicate 100% of cardiac surgical patients (n=2615; mean age 60.8.7 yrs) in their unit develop SIRS during the first 12 hours of post-operative care (Michalopoulos et al., 2005, Intensive Care Medicine 22(1):S134). Recent research has suggested that because of the amount of cellular damage (necrosis) from major physical injury and trauma, mitochondrial proteins are released into circulation and stimulate damage-associated molecular patterns (DAMPs). This is significant as mitochondria are cellular organelles which were originally derived from bacteria via a process known as evolutionary endosymbiosis. It is these DAMPS that stimulate an acute phase response by the innate immune system that is biologically concordant with pathogen-associated molecular patterns (PAMPs) released during infection (Zhang et al., 2010, Nature 464:104-107).

If infection is suspected in addition to the any of the above SIRS clinical presentations, the term sepsis is applied. Sepsis can be defined as a systemic inflammatory response to infection; typically a Gram negative or Gram positive bacterial or fungal infection. However, microbiological evidence of a circulating pathogen is not necessary to confirm the diagnosis of sepsis. Severe sepsis includes hypotension and evidence of organ dysfunction. When hypotension cannot be managed with intravenous fluids, the diagnosis of septic shock is applied (Bone et al., 1992, Chest 101:1644-55; American College of Chest Physicians/Society of Critical Care Medicine Consensus Conference. Definitions of sepsis and organ failure and guidelines for the use of innovative therapies in sepsis. 1992, Crit Care Med. 20(6):864-874; Bernard et al., (PROWESS Study Group), 2001, N Engl J Med. 344(10):699-709). It was thus recommended at the 1991 Consensus Conference that, when patients are identified as having SIRS or multiple organ dysfunction syndrome (MODS), sequential (i.e., daily or more frequently) risk stratification or probability estimate techniques should be applied to describe the course of the syndrome (Bone et al., 1992, supra; American College of Chest Physicians/Society of Critical Care Medicine Consensus Conference, 1992, supra).

Sepsis is a life-threatening disorder and the leading cause of mortality in the adult intensive care unit (ICU) ranging from between 18-50% (Sundararajan et al., 2005, Crit. Care Med. 33:71-80; Finfer et al., 2004, Care Med. 30:589-596; Martin et al., 2003, N Engl J. Med. 348:1546-1554; Australian Institute of Health & Welfare, Canberra (2006). Mortality over the twentieth century in Australia. Trends and patterns in major causes of death. Mortality Surveillance Series, Number 4, p49). In developed countries, the incidence of sepsis is expected to rise due to aging populations, immune-compromised patients (e.g., patients on chemotherapy, or have had a transplant or are on chronic corticosteroids), increasing longevity of patients with chronic diseases, antimicrobial resistance, especially in younger people, as well as viral illnesses such as AIDS.

Antimicrobial resistance is becoming a significant problem in critical care patient management, particularly with Gram-negative bacilli (Hotchkiss and Donaldson. 2006, Nature Reviews Immunology 6:813-822; Eber et al., 2010, Arch Intern Med. 170(4):374-353). Recent evidence suggests that indiscriminate use of antibiotics has contributed to resistance and hence, guidance on antibiotic treatment duration is now imperative in order to reduce consumption in tertiary care ICU settings (Hanberger et al., 1999, JAMA. 281:61-71).

Approximately 20 million cases of severe sepsis arise globally per annum, and account for up to 135,000 deaths in Europe and 215,000 in the USA (Neuhauser et al., 2003, JAMA. 289:885-888).

While half of these infections are estimated to be community-acquired in the United States, research suggests that the other half relate to hospital acquired infections (HAI), which account for increased hospital in-patient admission by as much as 14 days, at an average cost of $46,000 per patient (Goldmann et al., 1996, JAMA. 275:234-40). Bacterial and fungal sepsis is a significant medical challenge not only in critical care but also for hematology, transplant, medical oncology and post-surgical in-patients.

Sepsis initiates a complex immunologic response that varies over time and is dependent on pre-existing co-morbidities. Although recent research demonstrates that both inflammatory and anti-inflammatory responses are occurring in this condition, during the early host response to microbial invasion, there is generally a hyperinflammatory signal. That is, the majority of the sepsis cases are the product of bacteria and fungi that do not ordinarily cause systemic disease in immunocompetent hosts. The local innate immune mechanisms essentially stimulate the release of cytokines, chemokines, prostanoids and leukotrienes that increase blood flow to local sources of infection and result in an influx of white blood cells. During this processes, toll-like receptors (TLRs) are also activated as part of the innate immune response and have direct anti-microbial activity in addition to influencing the antigen-specific adaptive response. TLRs are a type of pattern recognition receptor that can identify PAMPs as soon as microbes breach dermal or intestinal barriers (Hotchkiss et al., 2009, Nature Medicine. 15(5):496-497). However, weaknesses in the innate host defence and release of endotoxins or other virulence factors can quickly lead to severe sepsis following a strong inflammatory response.

For many decades, the cornerstone of sepsis diagnosis and treatment has been identifying the causative circulating pathogen and quantitating single immune-related blood analytes—medical determinants which are not necessarily specific to sepsis, but routinely conducted to assess the patient's physiological response to the pathogen. Currently, the gold standard for detection of bacteria and fungi is blood culture in microbiological media with the aim of growing the causative organism. This method typically requires between 48-72 hours of incubation before the microbe can be identified and antibiotic sensitivity provided, such that evidence-based treatment can be implemented in comparison to the initial empiric practices. In contrast, it has recently been proposed by Hotchkiss et al. (Adib-Conquy et al, 2009, Thromb Haemast. 101(1):36-47) that the development of sepsis represents the harmful consequences of an exuberant innate immune response. While most patients survive this “hyperinflammatory phase,” it was suggested that what follows is a stage of protracted immunosuppression that is referred to as immunoparalysis (Monneret et al, 2008, Mol Med. 14(1-2):64-78; Wade et al, 2009, Science 326:865-876). This secondary immunosuppression has been characterized by the loss of delayed type hypersensitivity response to positive control antigens, failure to clear the primary infection and development of secondary infections which can include activation of normally latent viruses such as CMV or HHV. Taken together, this implies that current clinical focus should be on enhancing/maintaining immune competence in critically ill patients. Thus, to achieve such a clinical goal there needs to be a method of monitoring the status of the immune system so that immunotherapy can be timed appropriately.

In terms of treatment and management plans; SIRS (also referred to herein as “infection-negative SIRS”) and sepsis are quite different. On initial presentation to the Emergency Department, a patient displaying two or more SIRS criteria will be treated with intravenous glucocorticosteroids (GCS) and antibiotics, even if infection is only suspected. Empiric treatment will continue until positive microbiology findings are known, past medical history is confirmed and/or there has been a positive clinical response to early management. If it is clear, based on clinical presentation and reason for admission, that the SIRS response is related to acute trauma, for example motor vehicle injury or an acute inflammatory condition such as anaphylaxis, the patient will be managed with other intravenous fluids, blood products or adrenaline, where indicated. However, it is important that a patient with a true SIRS response is definitively managed as early as possible so to conserve antibiotic efficacy. Likewise, it is essential that a patient with a bacterial or fungal infection be managed with antibiotics and not steroids so that immune function is not compromised. Differential diagnosis is exponentially more difficult when a patient presents to the Emergency Department with clinically significant changes to vital signs such as heart rate and blood pressure in addition to a fever. These are signs and symptoms of the early stages of infection-negative SIRS and infection-positive SIRS (sepsis), and impossible to delineate the two conditions clinically. However, although the two conditions can be separated based on physiological endpoints, the molecular biology is considered only capable of identifying changes in the chemical signatures that appear when a severe infection is developing.

At the present time, diagnostic practices in clinical pathology are moving toward gene-protein-metabolite targeted pathways, as novel molecular profiles offer the opportunity to assess discrete yet unique changes in multiple biomarkers in a matter of hours, and potentially minutes. The combination of high specificity and sensitivity, low contamination risk and blood collection, as well as processing speed has made molecular techniques, such as quantitative real time PCR (qRT PCR) technology, an efficient alternative in comparison to microbiological culture.

Given that the majority of patients (>80%) admitted to the tertiary care ICU setting have SIRS of varying etiologies, including following major surgery, it is of enormous clinical importance that those patients who have a suspected infection or are at high risk of infection can be identified early and be graded and monitored, in order to initiate evidence-based and goal-orientated medical therapy. This is critical, as the acute management plans for SIRS with and without infection are very different. Dependence on empiric treatment means that some patients may be receiving excessive antibiotics while others are receiving treatment (e.g. corticosteroids) that is immuno-suppressive because a clear site of infection has not been identified. Furthermore, once patients are identified as having sepsis, regular monitoring of the immune system is considered essential for clinicians to modulate therapy dependent on immune system status, the type of infection and multi-organ complications that may be associated with sepsis.

### SUMMARY OF THE INVENTION

The present invention arises from the unexpected discovery that the range of transcripts expressed from certain individual genes in peripheral blood varies between patients with sepsis, patients with infection-negative SIRS (also referred to herein as “inSIRS”) and patients following major surgery. In particular, the present inventors have found that certain exons of individual genes are differentially expressed in peripheral blood between these conditions (also referred to herein as “condition-separating exons”) whilst others from the same genes are not so differential expressed. Based on this discovery, the present inventors have developed various methods and kits, which take advantage of condition-separating exons to detect the presence, absence or risk of development of sepsis, inSIRS and systemic inflammation following major surgery. In certain embodiments, these assays and kits represent a significant advance over prior art assays and kits which have not been able to distinguish between systemic inflammation following major surgery and infection-negative SIRS. Accordingly, in these embodiments, the present invention provides a means to separate these two groups from themselves and from sepsis allowing for qualitative or quantitative grading of inflammatory response as if there were a “continuum” of severity of inflammatory response from post-surgical inflammation through to sepsis.

The present invention thus represents a significant advance over current technologies for the management of sepsis, infection negative SIRS and post-surgical inflammation. In certain advantageous embodiments, it relies upon measuring the level of certain markers in cells, especially circulating leukocytes, of the host. In some embodiments where circulating leukocytes are the subject of analysis, it is proposed that detection of the presence or absence of a host response to sepsis and its sequelae (also referred to herein as “sepsis-related conditions”) will be feasible at very early stages of its progression before extensive tissue damage has occurred.

The present invention addresses the problem of distinguishing between sepsis, infection-negative SIRS and post-surgical inflammation by detecting a host response that may be measured in host cells. Advantageous embodiments involve monitoring the expression of particular gene transcripts in peripheral leukocytes of the immune system, which may be reflected in changing patterns of RNA levels or protein production that correlate with the presence of active disease or response to disease.

Accordingly, in one aspect, the present invention provides methods for assessing whether a subject has, or is at risk of developing, one of a plurality of conditions selected from sepsis, infection-negative SIRS (hereafter referred to as “inSIRS”) and post-surgical inflammation. These methods generally comprise comparing the level of at least one expression product (also referred to herein as an “inflammatory response continuum” (IRC) marker expression product”) of a multi-transcript-producing gene in the subject to the level of a corresponding IRC marker expression product in at least one control subject selected from: a post-surgical inflammation-positive subject, an inSIRS positive subject, a sepsis-positive subject and a normal subject, wherein a difference between the level of the at least one IRC marker expression product and the level of the corresponding IRC marker expression product indicates whether the subject has, or is at risk of developing, one of the conditions, wherein the at least one IRC marker expression product is predetermined as being differentially expressed between at least two of the conditions and wherein at least one other expression product from the multi-transcript producing gene is predetermined as being not so differentially expressed. The at least one ICR marker expression product is suitably selected from an ICR marker transcript or an ICR marker polypeptide.

In some embodiments, the multi-transcript-producing gene is selected from the group consisting of: ankyrin repeat and death domain containing 1A (ANKDD1A) gene, rho 2 (GABRR2) gene, orthodenticle homeobox 1 (OTX1) gene, pannexin 2 (PANX2) gene, rhomboid 5 homolog 2 (Drosophila) (RHBDF2) gene, SLAM family member 7 (SLAMF7) gene, autophagy/beclin-1 regulator 1 (AMBRA1) gene, carboxylesterase 2 (intestine, liver) (CES2) gene, caseinolytic peptidase B homolog (E. coli) (CLPB) gene, homeodomain interacting protein kinase 2 (HIPK2) gene and chromosome 1 open reading frame 91 (C1ORF91) gene, N-deacetylase/N-sulfotransferase (heparan glucosaminyl) 1 (NDST1) gene, solute carrier family 36 (proton/amino acid symporter) (member 1 (SLC36A1) gene, ADAM metallopeptidase domain 19 (meltrin beta) (ADAM19) gene, cullin 7 (CUL7) gene, thyroglobulin (TG) gene, programmed cell death 1 ligand 2 (PDCD1LG2) gene, glutamate receptor (ionotropic (N-methyl D-aspartate-like 1A (GRINL1A) gene, mahogunin (ring finger 1 (MGRN1) gene, syntrophin (beta 2 (dystrophin-associated protein A 1 (59 kDa (basic component 2) (SNTB2) gene, cyclin-dependent kinase 5 (regulatory subunit 1 (p35) (CDK5R1) gene, glucosidase (alpha; acid (GAA) gene, katanin p60 subunit A-like 2 (KATNAL2) gene, carcinoembryonic antigen-related cell adhesion molecule 4 (CEACAM4) gene, zinc finger protein 335 (ZNF335) gene, aspartate beta-hydroxylase domain containing 2 (ASPHD2) gene, acidic repeat containing (ACRC) gene, butyrophilin-like 3/butyrophilin-like 8 (BTNL3, BTLN8) gene, Moloney leukemia virus 10 homolog (mouse) (MOVE)) gene, mediator complex subunit 12-like (MED12L) gene, kelch-like 6 (Drosophila) (KLHL6) gene, PDZ and LIM domain 5 (PDLIM5) gene, UDP-N-acetyl-alpha-D-galactosamine:polypeptide N-acetylgalactosaminyltransferase 10 (GALNT10) gene, secernin 1 (SCRN1) gene, vesicular (overexpressed in cancer (prosurvival protein 1 (VOPP1, RP11-289I10.2) gene, FK506 binding protein 9, 63 kDa (FKBP9, FKBP9, FKBP9L, AC091812.2) gene, kinesin family member 27 (KIF27) gene, piwi-like 4 (Drosophila) (PIWIL4) gene, telomerase-associated protein 1 (TEP1) gene, GTP cyclohydrolase 1, (GCH1) gene, proline rich 11, (PRR11) gene, cadherin 2, type 1, N-cadherin (neuronal) (CDH2) gene, protein phosphatase 1B-like (FLJ40125, AC138534.1) (PPMIN) gene, related RAS viral (r-ras) oncogene homolog, (RRAS) gene, dolichyl-diphosphooligosaccharide-protein glycosyltransferase, (DDOST) gene, anterior pharynx defective 1 homolog A (C. elegans) (APH1A) gene, tubulin tyrosine ligase (TTL) gene, testis expressed 261, (TEX261) gene, coenzyme Q2 homolog, prenyltransferase (yeast) (COQ2) gene, FCH and double SH3 domains 1, (FCHSD1) gene, BCL2-antagonist/killer 1, (BAK1) gene, solute carrier family 25 (mitochondrial carrier; phosphate carrier) member 25, (SLC25A25) gene, RELT tumor necrosis factor receptor, (RELT) gene, acid phosphatase 2, lysosomal, (ACP2) gene, TBC1 domain family, member 2B, (TBC1D2B) gene, Fanconi anemia, complementation group A, (FANCA) gene, solute carrier family 39 (metal ion transporter) member 11, (SLC39A11) gene.

In some embodiments, the methods comprise comparing the level of at least one IRC marker transcript to the level of a corresponding IRC marker transcript, wherein the IRC marker transcript is selected from the group consisting of: (a) a polynucleotide comprising a nucleotide sequence that shares at least 70% (or at least 71% to at least 99% and all integer percentages in between) sequence identity with the sequence set forth in any one of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513 or 515, or a complement thereof; (b) a polynucleotide comprising a nucleotide sequence that encodes a polypeptide comprising the amino acid sequence set forth in any one of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98; 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514 or 516; (c) a polynucleotide comprising a nucleotide sequence that encodes a polypeptide that shares at least 70% (or at least 71% to at least 99% and all integer percentages in between) sequence similarity or identity with at least a portion of the sequence set forth in SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514 or 516; (d) a polynucleotide expression product comprising a nucleotide sequence that hybridizes to the sequence of (a), (b), (c) or a complement thereof, under at least medium or high stringency conditions.

In some embodiments, the methods comprise comparing the level of at least one IRC marker polypeptide to the level of a corresponding IRC marker polypeptide, wherein the IRC marker polypeptide is selected from the group consisting of: (i) a polypeptide comprising the amino acid sequence set forth in any one of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514 or 516; and (ii) a polypeptide comprising an amino acid sequence that shares at least 70% (or at least 71% to at least 99% and all integer percentages in between) sequence similarity or identity with the sequence set forth in any one of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260; 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514 or 516.

In some embodiments, the methods comprise: (1) measuring in a biological sample obtained from the subject the level of the at least one IRC marker expression product and (2) comparing the measured level of each IRC marker expression product to the level of a corresponding IRC marker expression product in a reference sample obtained from the at least one control subject. In illustrative examples of this type, the methods comprise assessing whether the subject has, or is at risk of developing, one of the plurality of conditions when the measured level of the or each IRC marker expression product is different than the measured level of the or each corresponding IRC marker expression product. In specific embodiments, the level of an individual IRC marker expression product is at least 110%, 120%, 130%, 140%, 150%, 160%, 170%, 180%, 190%, 200%, 300%, 400%, 500%, 600%, 700%, 800%, 900% or 1000%, or no more than about 95%, 90%, 80%, 70%, 60%, 50%, 40%, 30%, 20%, 10%, 5%, 4%, 3%, 2%, 1%, 0.5%, 0.1%, 0.01%, 0.001% or 0.0001% of the level of an individual corresponding IRC expression product, which is hereafter referred to as “differential expression.”

In some embodiments, the presence or risk of development of sepsis is determined by detecting in the subject a decrease in the level of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 IRC marker expression products from a multi-transcript-producing gene selected from the group consisting of: KIF27, OTX1, CDK5R1, FKBP9, CDH2, ADAM19, BTNL3/8 and PANX2 (hereafter referred to as “LIST A”), as compared to the level of a corresponding IRC marker expression product(s) in a post-surgical inflammation-positive control subject. In some embodiments, the presence or risk of development of post-surgical inflammation is determined by detecting in the subject an increase in the level of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 IRC marker expression product(s) from at least one multi-transcript-producing gene selected from the group consisting of KIF27, OTX1, CDK5R1, FKBP9, CDH2, ADAM19, BTNL3/8 and PANX2(i.e., LIST A), as compared to the level of a corresponding IRC marker expression product in a sepsis control subject. In illustrative examples of these embodiments, the KIF27 IRC marker expression product comprises a nucleotide sequence corresponding to an exon selected from KIF27 exon 4 and exon 7, or an amino acid sequence encoded by that exon. Representative KIF27 IRC transcripts are set forth in SEQ ID NO: 1, 3, 5, 7 and 9 and representative KIF27 IRC polypeptides are set forth in SEQ ID NO: 2, 4, 6, 8, and 10. In other illustrative examples, the OTX1 IRC marker expression product comprises a nucleotide sequence corresponding to OTX1 exon 5 or an amino acid sequence encoded by that exon. Representative OTX1 IRC transcripts are set forth in SEQ ID NO: 11 and 13 and representative OTX1 IRC polypeptides are set forth in SEQ ID NO: 12 and 14. In still other illustrative examples, the CDK5R1 IRC marker expression product comprises a nucleotide sequence corresponding to CDK5R1 exon 2, or an amino acid sequence encoded by that exon. A representative CDK5R1 IRC transcript is set forth in SEQ ID NO: 15 and a representative CDK5R1 IRC polypeptide is set forth in SEQ ID NO: 16. In still other illustrative examples, the FKBP9 IRC marker expression product comprises a nucleotide sequence corresponding to FKBP9 exon 10, or amino acid sequence(s) encoded by that exon. A representative FKBP9 IRC transcript is set forth in SEQ ID NO: 17 and a representative FKBP9 IRC polypeptide is set forth in SEQ ID NO: 18. In still other illustrative examples, the CDH2 IRC marker expression product comprises a nucleotide sequence corresponding to CDH2 exon 10, or an amino acid sequence encoded by that exon. Representative CDH2 IRC transcripts are set forth in SEQ ID NO: 19 and 21, and representative CDH2 IRC polypeptides are set forth in SEQ ID NO: 20 and 22. In still other illustrative examples, the ADAM19 IRC marker expression product comprises a nucleotide sequence corresponding to ADAM19 exon 10, or an amino acid sequence encoded by that exon. Representative ADAM19 IRC transcripts are set forth in SEQ ID NO: 23, 25, 27 and 29, and representative ADAM19 IRC polypeptides are set forth in SEQ ID NO: 24, 26, 28 and 30. In still other illustrative examples, the BTNL8/3 IRC marker expression product comprises a nucleotide sequence corresponding to BTNL8/3 exon 6, or an amino acid sequence encoded by that exon. Representative BTNL8/3 IRC transcripts are set forth in SEQ ID NO: 31, 33, 35, 37, 39 and 41, and representative BTNL8/3 IRC polypeptides are set forth in SEQ ID NO: 32, 34, 36, 38, 40 and 42. In other illustrative examples, the PANX2 IRC marker expression product comprises a nucleotide sequence corresponding to an exon selected from PANX2 exon 1 and exon 2, or an amino acid sequence encoded by that exon. Illustrative PANX2 IRC transcripts are set forth in SEQ ID NO: 43, 45 and 47 and illustrative PANX2 IRC polypeptides are set forth in SEQ ID NO: 44, 46 and 48. Information on each gene in LIST A exhibiting splice variation and ability to determine the presence or risk of sepsis versus post-surgical inflammation, the corresponding sequence numbers, log fold changes (and direction), T adjusted P value, relevant exon number and number of possible exons in the gene, is presented in Table 7.

In some embodiments, the presence or risk of development of sepsis is determined by detecting in the subject an increase in the level of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157 or 158 IRC marker expression product(s) from at least one multi-transcript-producing gene selected from the group consisting of: PDLIM5, SCRN1, ASPHD2, VOPP1, ACRC, GALNT10, AC1385341, MED12L, RHBDF2, KLHL6, TEP1, PIWIL6, PRR1, RRAS, TG, ANKDD1A, GABRR2, MOV10, SLAMF7, PDCDILG2 and GCH1 (hereafter referred to as “LIST B”), as compared to the level of a corresponding IRC marker expression product in a post-surgical-positive subject control subject. In some embodiments, the presence or risk of development of post-surgical inflammation is determined by detecting in the subject a decrease in the level of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157 or 158 IRC marker expression product(s) from at least one multi-transcript-producing gene selected from the group consisting of: PDLIM5, SCRN1, ASPHD2, VOPP1, ACRC, GALNT10, AC1385341, MED12L, RHBDF2, KLHL6, TEP1, PIWIL6, PRR1, RRAS, TG, ANKDD1A, GABRR2, MOV10, SLAMF7, PDCDILG2 and GCH1 (i.e., LIST B), as compared to the level of a corresponding IRC marker expression product in a sepsis control subject. In illustrative examples of these embodiments, the PDLIM5 IRC marker expression product comprises a nucleotide sequence corresponding to PDLIM5 exon 5 or an amino acid sequence encoded by that exon. A non-limiting PDLIM5 IRC transcript is set forth in SEQ ID NO: 49 and a non-limiting PDLIM5 IRC polypeptide is set forth in SEQ ID NO: 50. In still other illustrative examples, the SCRN1 IRC marker expression product comprises a nucleotide sequence corresponding to SCRN1 exon 5 or an amino acid sequence encoded by that exon. Representative SCRN1 IRC transcripts are set forth in SEQ ID NO: 51, 53, 55, 57, 59, 61 and 63, and representative SCRN1 IRC polypeptides are set forth in SEQ ID NO: 52, 54, 56, 58, 60, 62 and 64. In still other illustrative examples, the ASPHD2 IRC marker expression product comprises a nucleotide sequence corresponding to ASPHD2 exon 4 or an amino acid sequence encoded by that exon. Representative ASPHD2 IRC transcripts are set forth in SEQ ID NO: 65, 67 and 69, and representative ASPHD2 IRC polypeptides are set forth in SEQ ID NO: 66, 68 and 70. In still other illustrative examples, the VOPP1 IRC marker expression product comprises a nucleotide sequence corresponding to VOPP1 exon 3 or an amino acid sequence encoded by that exon. Representative VOPP1 IRC transcripts are set forth in SEQ ID NO: 71, 73, 75, 77, 79, 81, 83, 85, 87, 89, 91 and 93, and representative VOPP1 IRC polypeptides are set forth in SEQ ID NO: 72, 74, 76, 78, 80, 82, 84, 86, 88, 90, 92 and 94. In still other illustrative examples, the ACRC IRC marker expression product comprises a nucleotide sequence corresponding to one or both exons selected from ACRC exons 3 and 5, or amino acid sequence(s) encoded by one or both of those exons. Non-limiting ACRC IRC transcripts are set forth in SEQ ID NO: 95 and 97, and non-limiting ACRC IRC polypeptides are set forth in SEQ ID NO: 96 and 98. In still other illustrative examples, the GALNT10 IRC marker expression product comprises a nucleotide sequence corresponding to GALNT10 exon 6 or an amino acid sequence encoded by that exon. Representative GALNT10 IRC transcripts are set forth in SEQ ID NO: 99 and 101, and representative GALNT10 IRC polypeptides are set forth in SEQ ID NO: 100 and 102. In still other illustrative examples, the AC1385341 IRC marker expression product comprises a nucleotide sequence corresponding to AC1385341 exon 3 or an amino acid sequence encoded by that exon. Representative AC1385341 IRC transcripts are set forth in SEQ ID NO: 103, 105, 107, 109, 111, 113, 115, 117, 119, 121 and 123, and representative AC1385341 IRC polypeptides are set forth in SEQ ID NO: 104, 106, 108, 110, 112, 114, 116, 118, 120, 122 and 124. In still other illustrative examples, the MED12L IRC marker expression product comprises a nucleotide sequence corresponding to MED12L exon 17 or an amino acid sequence encoded by that exon. Representative MED12L IRC transcripts are set forth in SEQ ID NO: 125 and 127, and representative MED12L IRC polypeptides are set forth in SEQ ID NO: 126 and 128. In still other illustrative examples, the RHBDF2 IRC marker expression product comprises a nucleotide sequence corresponding to an exon selected from RHBDF2 exons 6, 9, 10, 11, 14, 17, 18 or 19, or an amino acid sequence encoded by that exon. Representative RHBDF2 IRC transcripts are set forth in SEQ ID NO: 129, 131 and 133 and representative RHBDF2 IRC polypeptides are set forth in SEQ ID NO: 130, 132 and 134. In still other illustrative examples, the KLHL6 IRC marker expression product comprises a nucleotide sequence corresponding to KLHL6 exon 7 or an amino acid sequence encoded by that exon. A representative KLHL6 IRC transcript is set forth in SEQ ID. NO: 135, and a representative KLHL6 IRC polypeptide is set forth in SEQ ID NO: 136. In other illustrative examples, the TEP1 IRC marker expression product comprises a nucleotide sequence corresponding to TEP1 exon 49, or an amino acid sequence encoded by that exon. Non-limiting TEP1 IRC transcripts are set forth in SEQ ID NO: 137 and 139, and non-limiting TEP1 IRC polypeptides are set forth in SEQ ID NO: 138 and 140. In still other illustrative examples, the PIWIL6 IRC marker expression product comprises a nucleotide sequence corresponding to one or both exons selected from PIWIL6 exons 2 and 14, or amino acid sequence(s) encoded by one or both of those exons. Non-limiting PIWIL6 IRC transcripts are set forth in SEQ ID NO: 141 and 143, and non-limiting PIWIL6 IRC polypeptides are set forth in SEQ ID NO: 142 and 144. In still other illustrative examples, the PRR11 IRC marker expression product comprises a nucleotide sequence corresponding to one or both exons selected from PRR11 exons 4 and 5, or amino acid sequence(s) encoded by one or both of those exons. A non-limiting PRR11 IRC transcript is set forth in SEQ ID NO: 145, and a non-limiting PRR11 IRC polypeptide is set forth in SEQ ID NO: 146. In still other illustrative examples, the RRAS IRC marker expression product comprises a nucleotide sequence corresponding to RRAS exon 1 or an amino acid sequence encoded by that exon. A representative BRAS IRC transcript is set forth in SEQ ID NO: 147, and a representative RRAS IRC polypeptide is set forth in SEQ ID NO: 148. In other illustrative examples, the TG IRC marker expression product comprises a nucleotide sequence corresponding to TG exon 6, or an amino acid sequence encoded by that exon. Non-limiting TG IRC transcripts are set forth in SEQ ID NO: 149 and 151, and non-limiting TG IRC polypeptides are set forth in SEQ ID NO: 150 and 152. In other illustrative examples, the ANKDD1A IRC marker expression product comprises a nucleotide sequence corresponding to ANKDD1A exon 7 or an amino acid sequence encoded by that exon. Non-limiting ANKDD1A IRC transcripts are set forth in SEQ ID NO: 153, 155, 157, 159 and 161 and non-limiting ANKDD1A IRC polypeptides are set forth in SEQ ID NO:154, 156, 158, 160 and 162. In other illustrative examples, the GABRR2 IRC marker expression product comprises a nucleotide sequence corresponding to an exon selected from GABRR2 exons 7, 8 or 9 or an amino acid sequence encoded by that exon. Illustrative GABRR2 IRC transcripts are set forth in SEQ ID NO: 163 and 165 and illustrative GABRR2 IRC polypeptides are set forth in SEQ ID NO: 164 and 166. In still other illustrative examples, the MOV10 IRC marker expression product comprises a nucleotide sequence corresponding to MOV10 exon 6 or an amino acid sequence encoded by that exon. Representative MOV10 IRC transcripts are set forth in SEQ ID NO: 167, 169, 171, 173, 175 and 177, and representative MOV10 IRC polypeptides are set forth in SEQ ID NO: 168, 170, 172, 174, 176 and 178. In still other illustrative examples, the SLAMF7 IRC marker expression product comprises a nucleotide sequence corresponding to an exon selected from SLAMF7 exons 2, 3, 4 or 5, or an amino acid sequence encoded by that exon. Non-limiting SLAMF7 IRC transcripts are set forth in SEQ ID NO: 179, 181, 183, 185, 187, 189, 191 and 193 and non-limiting SLAMF7 IRC polypeptides are set forth in SEQ ID NO: 180, 182, 184, 186, 188, 190, 192, and 194. In still other illustrative examples, the PDCILG2 IRC marker expression product comprises a nucleotide sequence corresponding to one or both exons selected from PDCILG2 exons 1 and 2, or amino acid sequence(s) encoded by one or both of those exons. Non-limiting PDCILG2 IRC transcripts are set forth in SEQ ID NO: 195 and 197, and non-limiting PDCILG2 IRC polypeptides are set forth in SEQ ID NO: 196 and 198. In still other illustrative examples, the GCH1 IRC marker expression product comprises a nucleotide sequence corresponding to GCH1 exon 2 or an amino acid sequence encoded by that exon. Representative GCH1 IRC transcripts are set forth in SEQ ID NO: 199, 201, 203 and 205, and representative GCH1 IRC polypeptides are set forth in SEQ ID NO: 1200, 202, 204 and 206. Information on each gene in LIST B exhibiting splice variation and ability to determine the presence or risk of sepsis versus post-surgical inflammation, the corresponding sequence numbers, log fold changes (and direction), T adjusted P value, relevant exon number and number of possible exons in the gene, is presented in Table 7.

In some embodiments, the presence or risk of development of sepsis is determined by detecting in the subject an increase in the level of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155 or 156 IRC marker expression product(s) from at least one multi-transcript-producing gene selected from the group consisting of: RELT, ACP2, FCHSD1, CLPB, SLC39A 1, TBC1D2B, APH1A, DDOST, BAK1, SLC25A25A, COQ2, FANCA, PIWIL4, ZNF335, TEX261, GABRR2, VOPP1, TTL, CES2, GALNT10, C1ORF91, AMBRA1 and SCRN1 (hereafter referred to as “LIST C”), as compared to the level of a corresponding IRC marker expression product in an inSIRS-positive control subject. In some embodiments, the presence or risk of development of inSIRS is determined by detecting in the subject a decrease in the level of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155 or 156 IRC marker expression(s) product from at least one multi-transcript-producing gene selected from the group consisting of: RELT, ACP2, FCHSD1, CLPB, SLC39A1, TBC1D2B, APH1A, DDOST, BAK1, SLC25A25A, COQ2, FANCA, PIWIL4, ZNF335, TEX261, GABRR2, VOPP1, TTL, CES2, GALNT10, C1ORF91, AMBRA1 and SCRN1 (i.e., LIST C), as compared to the level of the corresponding IRC marker expression product in a sepsis-positive control subject. In illustrative examples of these embodiments, the RELT IRC marker expression product comprises a nucleotide sequence corresponding to RELT exon 4 or an amino acid sequence encoded by that exon. Illustrative RELT IRC transcripts are set forth in SEQ ID NO: 307 and 209 and illustrative RELT IRC polypeptides are set forth in SEQ ID NO: 208 and 210. In other illustrative examples, the ACP2 IRC marker expression product comprises a nucleotide sequence corresponding to ACP2 exon 7 or an amino acid sequence encoded by that exon. A non-limiting ACP2 IRC transcript is set forth in SEQ ID NO: 211 and a non-limiting ACP2 IRC polypeptide is set forth in SEQ ID NO: 212. In still other illustrative examples, the FCHSD1 IRC marker expression product comprises a nucleotide sequence corresponding to FCHSD1 exon 14 or an amino acid sequence encoded by that exon. Illustrative FCHSD1 IRC transcripts are set forth in SEQ ID NO: 213 and 215 and illustrative FCHSD1 IRC polypeptides are set forth in SEQ ID NO: 214 and 216. In still other illustrative examples, the CLPB IRC marker expression product comprises a nucleotide sequence corresponding to CLPB exon 10 or an amino acid sequence encoded by that exon. Representative CLPB IRC transcripts are set forth in SEQ ID NO: 217, 219 and 221 and representative CLPB IRC polypeptides are set forth in SEQ ID NO: 218, 220 and 222. In other illustrative examples, the SLC39A11 IRC marker expression product comprises a nucleotide sequence corresponding to SLC39A11 exon 2 or an amino acid sequence encoded by that exon. A non-limiting SLC39A11 IRC transcript is set forth in SEQ ID NO: 223 and a non-limiting SLC39A11 IRC polypeptide is set forth in SEQ ID NO: 224. In other illustrative examples, the TBC1D2B IRC marker expression product comprises a nucleotide sequence corresponding to TBC1D2B exon 13 or an amino acid sequence encoded by that exon. Illustrative TBC1D2B IRC transcripts are set forth in SEQ ID NO: 225, 227 and 229 and illustrative TBC1D2B IRC polypeptides are set forth in SEQ ID NO: 226, 228 and 230. In still other illustrative examples, the APH1A IRC marker expression product comprises a nucleotide sequence corresponding to APH1A exon 1 or an amino acid sequence encoded by that exon. Illustrative APH1A IRC transcripts are set forth in SEQ ID NO: 231, 233, 235, 237, 239 and 241 and illustrative APH1A IRC polypeptides are set forth in SEQ ID NO: 232, 234, 236, 238, 240 and 242. In other illustrative examples, the DDOST IRC marker expression product comprises a nucleotide sequence corresponding to DDOST exon 2 or an amino acid sequence encoded by that exon. A non-limiting DDOST IRC transcript is set forth in SEQ ID NO: 243 and a non-limiting DDOST IRC polypeptide is set forth in SEQ ID NO: 244. In still other illustrative examples, the BAK1 IRC marker expression product comprises a nucleotide sequence corresponding to BAK1 exon 7 or an amino acid sequence encoded by that exon. Illustrative BAK1 IRC transcripts are set forth in SEQ ID NO: 245 and 247 and illustrative BAK1 IRC polypeptides are set forth in SEQ ID NO: 246 and 248. In still other illustrative examples, the SLC25A25A IRC marker expression product comprises a nucleotide sequence corresponding to SLC25A25A exon 10 or an amino acid sequence encoded by that exon. Illustrative SLC25A25A IRC transcripts are set forth in SEQ ID NO: 249, 251, 253, 255, 257, 259 and 261 and illustrative. SLC25A25A IRC polypeptides are set forth in SEQ ID NO: 250, 252, 254, 256, 258, 260 and 262. In still other illustrative examples, the COQ1 IRC marker expression product comprises a nucleotide sequence corresponding to COQ1 exon 1 or an amino acid sequence encoded by that exon. Illustrative COQ1 IRC transcripts are set forth in SEQ ID NO: 263, 265 and 267 and illustrative COQ1 IRC polypeptides are set forth in SEQ ID NO: 264, 266 and 268. In still other illustrative examples, the FANCA IRC marker expression product comprises a nucleotide sequence corresponding to FANCA exon 35 or an amino acid sequence encoded by that exon. Illustrative FANCA IRC transcripts are set forth in SEQ ID NO: 269 and 271 and illustrative FANCA IRC polypeptides are set forth in SEQ ID NO: 270 and 272. In other illustrative examples, the PIWIL4 IRC marker expression product comprises a nucleotide sequence corresponding to one or both exons selected from PIWIL4 exons 2 and 14, or amino acid(s) sequence encoded by one or both of those exons. Non-limiting PIWIL4 IRC transcripts are set forth in SEQ ID NO: 273 and 275 and non-limiting PIWIL4 IRC polypeptides are set forth in SEQ ID NO: 274 and 276. In still other illustrative examples, the ZNF335 IRC marker expression product comprises a nucleotide sequence corresponding to ZNF335 exon 5 or an amino acid sequence encoded by that exon. Illustrative ZNF335 IRC transcripts are set forth in SEQ ID NO: 277, 279 and 281 and illustrative ZNF335 IRC polypeptides are set forth in SEQ ID NO: 278, 280 and 282. In still other illustrative examples, the TEX261 IRC marker expression product comprises a nucleotide sequence corresponding to TEX261 exon 3 or an amino acid sequence encoded by that exon. Illustrative TEX261 IRC transcripts are set forth in SEQ ID NO: 283 and 285 and illustrative TEX261 IRC polypeptides are set forth in SEQ ID NO: 284 and 286. In other illustrative examples, the GABRR2 IRC marker expression product comprises a nucleotide sequence corresponding to 1, 2 or each of the exons selected from GABRR2 exons 7, 8 and 9, or amino acid sequence(s) encoded by 1, 2 or each of those exons. Non-limiting GABRR2 IRC transcripts are set forth in SEQ ID NO: 287 and 289 and non-limiting GABRR2 IRC polypeptides are set forth in SEQ ID NO: 288 and 290. In still other illustrative examples, the VOPP1 IRC marker expression product comprises a nucleotide sequence corresponding to VOPP1 exon 3 or an amino acid sequence encoded by that exon. Illustrative VOPP1 IRC transcripts are set forth in SEQ ID NO: 291, 293, 295, 297, 299, 301, 303, 305, 307, 309, 311 and 313 and illustrative VOPP1 IRC polypeptides are set forth in SEQ ID NO: 292, 294, 296, 298, 300, 302, 304, 306, 308, 310, 312 and 314. In other illustrative examples, the TTL IRC marker expression product comprises a nucleotide sequence corresponding to TTL exon 7 or an amino acid sequence encoded by that exon. A non-limiting TTL IRC transcript is set forth in SEQ ID NO: 315 and a non-limiting TTL IRC polypeptide is set forth in SEQ ID NO: 316. In other illustrative examples, the CES2 IRC marker expression product comprises a nucleotide sequence corresponding to CES2 exon 1 or an amino acid sequence encoded by that exon. Illustrative CES2 IRC transcripts are set forth in SEQ ID NO: 317 and 319 and illustrative CES2 IRC polypeptides are set forth in SEQ ID NO: 318 and 320. In still other illustrative examples, the GALNT10 IRC marker expression product comprises a nucleotide sequence corresponding to GALNT10 exon 6 or an amino acid sequence encoded by that exon. Illustrative GALNT10 IRC transcripts are set forth in SEQ ID NO: 321 and 323 and illustrative GALNT10 IRC polypeptides are set forth in SEQ ID NO: 322 and 324. In still other illustrative examples, the C1ORF91 IRC marker expression product comprises a nucleotide sequence corresponding to C1ORF91 exon 2 or an amino acid sequence encoded by that exon. Illustrative C1ORF91 IRC transcripts are set forth in SEQ ID NO: 325, 327, 329, 331, 333 and 335 and illustrative C1ORF91 IRC polypeptides are set forth in SEQ ID NO: 326, 328, 330, 332, 334 and 336. In other illustrative examples, the AMBRA1 IRC marker expression product comprises a nucleotide sequence corresponding to an exon selected from AMBRA1 exons 2 and 4, or an amino acid sequence encoded by that exon. Non-limiting AMBRA1 IRC transcripts are set forth in SEQ ID NO: 337, 339, 341, 343, 345 and 347 and non-limiting AMBRA1 IRC polypeptides are set forth in SEQ ID NO: 338, 340, 342, 344, 346 and 348. In still other illustrative examples, the SCRN1 IRC marker expression product comprises a nucleotide sequence corresponding to SCRN1 exon 5 or an amino acid sequence encoded by that exon. Illustrative SCRN1 IRC transcripts are set forth in SEQ ID NO: 349, 351, 353, 355, 357, 359 and 361 and illustrative SCRN1 IRC polypeptides are set forth in SEQ ID NO: 350, 352, 354, 356, 358, 360 and 362. Information on each gene in LIST C exhibiting splice variation and ability to determine the presence or risk of sepsis versus post-surgical inflammation, the corresponding sequence numbers, log fold changes (and direction), T adjusted P value, relevant exon number and number of possible exons in the gene, is presented in Table 8.

In some embodiments, the presence or risk of development of sepsis is determined by detecting in the subject an decrease in the level of at least 1, 2, 3, 4, 5, 6, 7, 8, 9, 10, 11, 12, 13, 14, 15, 16, 17, 18, 19 or 20 IRC marker expression product(s) from at least one multi-transcript-producing gene selected from the group consisting of: GRINL1A and KATNAL2 (hereafter referred to as “LIST D”), as compared to the level of a corresponding IRC marker expression product in an inSIRS-positive control subject. In some embodiments, the presence or risk of development of inSIRS is determined by detecting in the subject a increase in the level of at least 1, 2, 3, 4, 5, 6, 7, 8, 9, 10, 11, 12, 13, 14, 15, 16, 17, 18, 19 or 20 IRC marker expression(s) product from at least one multi-transcript-producing gene selected from the group consisting of: GRINL1A and KATNAL2 (i.e., LIST D), as compared to the level of the corresponding IRC marker expression product in a sepsis-positive control subject. In illustrative examples of these embodiments, the GRINL1 IRC marker expression product comprises a nucleotide sequence corresponding to an exon selected from GRINL1 exon 5, or an amino acid sequence encoded by that exon. Non-limiting GRINL1 IRC transcripts are set forth in SEQ ID NO: 363, 365, 367, 369, 371, 373, 375 and 377 and non-limiting GRINL1 IRC polypeptides are set forth in SEQ ID NO:364, 366, 368, 370, 372, 374, 376 and 378. In other illustrative examples, the KATNAL2 IRC marker expression product comprises a nucleotide sequence corresponding to KATNAL2 exon 3 or an amino acid sequence encoded by that exon. Illustrative KATNAL2 IRC transcripts are set forth in SEQ ID NO: 379 and 381 and illustrative KATNAL2 IRC polypeptides are set forth in SEQ ID NO: 380 and 382. Information on each gene in LIST D exhibiting splice variation and ability to determine the presence or risk of sepsis versus post-surgical inflammation, the corresponding sequence numbers, log fold changes (and direction), T adjusted P value, relevant exon number and number of possible exons in the gene, is presented in Table 8.

In some embodiments, the presence or risk of development of inSIRS is determined by detecting in the subject an increase in the level of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38, IRC marker expression product(s) from at least one multi-transcript-producing gene selected from the group consisting of: PDCD1LG2, KATNAL2, GRINL1A, ACRC, TG, and ASPHD2 (hereafter referred to as “LIST E”), as compared to the level of a corresponding IRC marker expression product in a post-surgical inflammation-positive control subject. In other embodiments, the presence or risk of development of post-surgical inflammation is determined by detecting in the subject a decrease in the level of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 IRC marker expression product(s) from at least one multi-transcript-producing gene selected from the group consisting of: PDCD1LG2, KATNAL2, GRINL1A, ACRC, TG, and ASPHD2 (i.e., LIST E), as compared to the level of a corresponding IRC marker expression product in an inSIRS-positive control subject. In illustrative examples of these embodiments, the PDCD1LG2 IRC marker expression product comprises a nucleotide sequence corresponding to PDCD1LG2 exon 1, 2 or an amino acid sequence encoded by those exons. Non-limiting PDCD1LG2 IRC transcripts are set forth in SEQ ID NO: 383 and 385 and non-limiting PDCD1LG21 IRC polypeptides are set forth in SEQ ID NO: 384 and 386. In other illustrative examples, the KATNAL2 IRC marker expression product comprises a nucleotide sequence corresponding to KATNAL2 exon 3 or an amino acid sequence encoded by that exon. Illustrative KATNAL2 IRC transcripts are set forth in SEQ ID NO: 387 and 389 and illustrative KATNAL2 IRC polypeptides are set forth in SEQ ID NO: 388 and 390. In other illustrative examples, the GRINL1 IRC marker expression product comprises a nucleotide sequence corresponding to an exon selected from GRINL1 exon 5, or an amino acid sequence encoded by that exon. Non-limiting GRINL1 IRC transcripts are set forth in SEQ ID NO: 391, 393, 395, 397, 399, 401, 403 and 405 and non-limiting GRINL1 IRC polypeptides are set forth in SEQ ID NO:392, 394, 396, 398, 400, 402, 404 and 406. n still other illustrative examples, the ACRC IRC marker expression product comprises a nucleotide sequence corresponding to one or both exons selected from ACRC exons 3 and 5, or amino acid sequence(s) encoded by one or both of those exons. Non-limiting ACRC IRC transcripts are set forth in SEQ ID NO: 407 and 409, and non-limiting ACRC IRC polypeptides are set forth in SEQ ID NO: 408 and 410. In other illustrative examples, the TG IRC marker expression product comprises a nucleotide sequence corresponding to TG exon 6, or an amino acid sequence encoded by that exon. Non-limiting TG IRC transcripts are set forth in SEQ ID NO: 411 and 413, and non-limiting TG IRC polypeptides are set forth in SEQ ID NO: 412 and 414. n still other illustrative examples, the ASPHD2 IRC marker expression product comprises a nucleotide sequence corresponding to ASPHD2 exon 4 or an amino acid sequence encoded by that exon. Representative ASPHD2 IRC transcripts are set forth in SEQ ID NO: 415, 417 and 419, and representative ASPHD2 IRC polypeptides are set forth in SEQ ID NO: 416, 418 and 420. Information on each gene in LIST E exhibiting splice variation and ability to determine the presence or risk of sepsis versus post-surgical inflammation, the corresponding sequence numbers, log fold changes (and direction), T adjusted P value, relevant exon number and number of possible exons in the gene, is presented in Table 9.

In some embodiments, the presence or risk of development of inSIRS is determined by detecting in the subject a decrease in the level of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95 or 96 IRC marker expression product(s) from at least one multi-transcript-producing gene selected from the group consisting of CUL7, BTNL8/3, PANX2, C1ORF91, ZNF335, MGRN1, GAA, CDK5R1, SNTB2, CLPB, ADAM19, SLC36A1, FKBP9, NDST1, HIPK2 and CEACAM4 (hereafter referred to as “LIST F”) as compared to the level of the corresponding IRC marker gene(s) in a post-surgical inflammation-positive control subject. In other embodiments, the presence or risk of development of post-surgical inflammation is determined by detecting in the subject an increase in the level of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95 or 96 IRC marker expression product(s) from at least one multi-transcript-producing gene selected from the group consisting of: CUL7, BTNL8/3, PANX2, C1ORF91, ZNF335, MGRN1, GAA, CDK5R1, SNTB2, CLPB, ADAM19, SLC36A1, FKBP9, NDST1, HIPK2 and CEACAM4 (i.e., LIST F) as compared to the level of the corresponding IRC marker gene(s) in an inSIRS-positive control subject. In non-limiting examples of these embodiments, the CUL7 IRC marker expression product comprises a nucleotide sequence corresponding to CUL7 exon 5 or an amino acid sequence encoded by that exon. An illustrative CUL7 IRC transcript is set forth in SEQ ID NO: 421 and an illustrative CUL7 IRC polypeptide is set forth in SEQ ID NO: 422. In illustrative examples, the HIPK2 IRC marker expression product comprises a nucleotide sequence corresponding to HIPK2 exon 11 or an amino acid sequence encoded by that exon. In still other illustrative examples, the BTNL8/3 IRC marker expression product comprises a nucleotide sequence corresponding to BTNL8/3 exon 6, or an amino acid sequence encoded by that exon. Representative BTNL8/3 IRC transcripts are set forth in SEQ ID NO: 423, 425, 427, 429, 431 and 433, and representative BTNL8/3 IRC polypeptides are set forth in SEQ ID NO: 424, 426, 428, 430, 432 and 434. In other illustrative examples, the PANX2 IRC marker expression product comprises a nucleotide sequence corresponding to an exon selected from PANX2 exon 1 and exon 2, or an amino acid sequence encoded by that exon. Illustrative PANX2 IRC transcripts are set forth in SEQ ID NO: 435, 437 and 439 and illustrative PANX2 IRC polypeptides are set forth In SEQ ID NO: 436, 438 and 440. In still other illustrative examples, the C1 ORF91 IRC marker expression product comprises a nucleotide sequence corresponding to C1ORF91 exon 2 or an amino acid sequence encoded by that exon. Illustrative C1ORF91 IRC transcripts are set forth in SEQ ID NO: 441, 443, 445, 447, 449 and 451 and illustrative C1ORF91 IRC polypeptides are set forth in SEQ ID NO: 442, 444, 446, 448, 450 and 452. In still other illustrative examples, the ZNF335 IRC marker expression product comprises a nucleotide sequence corresponding to ZNF335 exon 5 or an amino acid sequence encoded by that exon. Illustrative ZNF335 IRC transcripts are set forth in SEQ ID NO: 453, 455 and 457 and illustrative ZNF335 IRC polypeptides are set forth in SEQ ID NO: 454, 456 and 458. In still other illustrative examples, the MGRN1 IRC marker expression product comprises a nucleotide sequence corresponding to MGRN1 exon 4 or an amino acid sequence encoded by that exon. Illustrative MGRN1 IRC transcripts are set forth in SEQ ID NO: 459, 461 and 463 and illustrative MGRN1 IRC polypeptides are set forth in SEQ ID NO: 460, 462 and 464. In still other illustrative examples, the GAA IRC marker expression product comprises a nucleotide sequence corresponding to GAA exon 3 or an amino acid sequence encoded by that exon. Illustrative GAA IRC transcripts are set forth in SEQ ID NO: 465, 467 and 469 and illustrative GAA IRC polypeptides are set forth in SEQ ID NO: 466, 468 and 470. In still other illustrative examples, the CDK5R1 IRC marker expression product comprises a nucleotide sequence corresponding to CDK5R1 exon 2 or an amino acid sequence encoded by that exon. An illustrative CDK5R1 IRC transcript is set forth in SEQ ID NO: 471, and an illustrative CDK5R1 IRC polypeptide is set forth in SEQ ID NO: 472. In still other illustrative examples, the SNTB2 IRC marker expression product comprises a nucleotide sequence corresponding to SNTB2 exon 4 or an amino acid sequence encoded by that exon. An illustrative SNTB2 IRC transcript is set forth in SEQ ID NO: 473, and an illustrative SNTB2 IRC polypeptide is set forth in SEQ ID NO: 474. In still other illustrative examples, the CLPB IRC marker expression product comprises a nucleotide sequence corresponding to CLPB exon 10, or an amino acid sequence encoded by that exon. Representative CLPB IRC transcripts are set forth in SEQ ID NO: 475, 477 and 479 and representative CLPB IRC polypeptides are set forth in SEQ ID NO: 476, 478 and 480. In still other illustrative examples, the ADAM19 IRC marker expression product comprises a nucleotide sequence corresponding to ADAM19 exon 10, or an amino acid sequence encoded by that exon. Representative ADAM19 IRC transcripts are set forth in SEQ ID NO: 481, 483, 485 and 487, and representative ADAM19 IRC polypeptides are set forth in SEQ ID NO: 482, 484, 486 and 488. In still other illustrative examples, the SLC36A1 IRC marker expression product comprises a nucleotide sequence corresponding to SLC36A1 exon 5, or an amino acid sequence encoded by that exon. Representative SLC36A1 IRC transcripts are set forth in SEQ ID NO: 489, 491, 493 and 495, and representative SLC36A1 IRC polypeptides are set forth in SEQ ID NO: 490, 492, 494 and 496. In still other illustrative examples, the FKBP9 IRC marker expression product comprises a nucleotide sequence corresponding to FKBP9 exon 10, or amino acid sequence(s) encoded by that exon. Representative FKBP9 IRC transcripts are set forth in SEQ ID NO: 497 and 499 and representative FKBP9 IRC polypeptides are set forth in SEQ ID NO: 498 and 500. In other illustrative examples, the CEACAM4 IRC marker expression product comprises a nucleotide sequence corresponding to 1, 2 or each of the exons selected from CEACAM4 exon 5, exon 7 and exon 23, or amino acid sequence(s) encoded by 1, 2 each of those exons. Illustrative CEACAM4 IRC transcripts are set forth in SEQ ID NO: 501 and 503, and illustrative CEACAM4 IRC polypeptides are set forth in SEQ ID NO: 502 and 504. Illustrative HIPK2 IRC transcripts are set forth in SEQ ID NO: 505, 507, 509, and 511 and illustrative HIPK2 IRC polypeptides are set forth in SEQ ID NO: 506, 508, 510 and 512. Information on each gene in LIST F exhibiting splice variation and ability to determine the presence or risk of sepsis versus post-surgical inflammation, the corresponding sequence numbers, log fold changes (and direction), T adjusted P value, relevant exon number and number of possible exons in the gene, is presented in Table 9.

In some embodiments, the methods comprise measuring the level of at least 1, 2, 3, 4, 5, 6, 7, 8, 9, 10, 11, 12, 13, 14, 15, 16, 17, 18, 19, 20, 21, 22, 23 or 24 individual IRC expression products of each of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 or 57 multi-transcript-producing genes (also referred to herein as “IRC multi-transcript-producing genes”). For example, the methods may comprise measuring the level of 1, 2, 3, 4, 5, 6, 7, 8, 9, 10, 11 or 12 IRC marker polynucleotides from an IRC multi-transcript-producing gene selected from: ANKDD1A, GABRR2, OTX1, PANX2, RHBDF2, SLAMF7, AMBRA1, CES2, CLPB, HIPK2, C1ORF91, NDST1, SLC36A1, ADAM19, CUL7, TG, PDCD1LG2, GRINL1A, MGRN1, SNTB2, CDK5R1, GAA, KATNAL2, CEACAM4, ZNF335, ASPHD2, ACRC, BTNL8, MOV10, MED12L, KLHL6, PDLIM5, GALNT10, SCRN1, VOPP1, FKBP9, KIF27, PIWIL4, TEP1, GCH1, PRR11, CDH2, PPM1N, RRAS, DDOST, APH1A, TTL, TEX261, COQ2, FCHSD1, BAK1, SLC25A25, RELT, ACP2, TBC1D2B, FANCA or SLC39A11, either alone or in combination with as much as 1, 2, 3, 4, 5, 6, 7, 8, 9, 10, 11 or 12 individual IRC marker polynucleotides from each of 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 19, 18, 17, 16, 15, 14, 13, 12, 11, 10, 9, 8, 7, 6, 5, 4, 3 or 2 IRC multi-transcript-producing genes or from 1 other IRC multi-transcript-producing gene. In other embodiments, the methods comprise measuring the level of 1, 2, 3, 4, 5, 6, 7, 8, 9, 10, 11 or 12 IRC marker polypeptides from an IRC multi-transcript-producing gene as defined herein, either alone or in combination with as much as 1, 2, 3, 4, 5, 6, 7, 8, 9, 10, 11 or 12 individual IRC marker polypeptides expressed from each of 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 19, 18, 17, 16, 15, 14, 13, 12, 11, 10, 9, 8, 7, 6, 5, 4, 3, 2 other IRC multi-transcript-producing genes or from 1 other IRC multi-transcript-producing gene.

In illustrative examples of this type, the methods further comprise detecting the level of at least one IRC marker expression product from two or more of LISTS A, B, C, D, E and F. In specific embodiments, the methods comprise detecting the level of at least one IRC marker expression product from one of the lists and the level of at least one different IRC marker expression product from another of the lists. In illustrative examples of this type, the methods comprise detecting the level of at least one IRC marker expression product from LIST A and the level of at least one other IRC marker expression product from LIST B. In other illustrative examples, the methods comprise detecting the level of at least one IRC marker expression product from LIST A and the level of at least one other IRC marker expression product from LIST C. In still other illustrative examples, the methods comprise detecting the level of at least one IRC marker expression product from LIST A and the level of at least one other IRC marker expression product from LIST D. In still other illustrative examples, the methods comprise detecting the level of at least one IRC marker expression product from LIST A and the level of at least one other IRC marker expression product from LIST E. In still other illustrative examples, the methods comprise detecting the level of at least one IRC marker expression product from LIST A and the level of at least one other IRC marker expression product from LIST F. In other illustrative examples, the methods comprise detecting the level of at least one IRC marker expression product from LIST B and the level of at least one other IRC marker expression product from LIST C. In still other illustrative examples, the methods comprise detecting the level of at least one IRC marker expression product from LIST B and the level of at least one other IRC marker expression product from LIST D. In still other illustrative examples, the methods comprise detecting the level of at least one IRC marker expression product from LIST B and the level of at least one other IRC marker expression product from LIST E. In still other illustrative examples, the methods comprise detecting the level of at least one IRC marker expression product from LIST B and the level of at least one other IRC marker expression product from LIST F. In other illustrative examples, the methods comprise detecting the level of at least one IRC marker expression product from LIST C and the level of at least one other IRC marker expression product from LIST D. In still other illustrative examples, the methods comprise detecting the level of at least one IRC marker expression product from LIST C and the level of at least one other IRC marker expression product from LIST E. In still other illustrative examples, the methods comprise detecting the level of at least one IRC marker expression product from LIST C and the level of at least one other IRC marker expression product from LIST F. In still other illustrative examples, the methods comprise detecting the level of at least one IRC marker expression product from LIST D and the level of at least one other IRC marker expression product from LIST E. In still other illustrative examples, the methods comprise detecting the level of at least one IRC marker expression product from LIST D and the level of at least one other IRC marker expression product from LIST F. In other illustrative examples, the methods comprise detecting the level of at least one IRC marker expression product from LIST E and the level of at least one other IRC marker expression product from LIST F.

In other embodiments, the methods comprise detecting the level of at least one IRC marker expression product from each of three lists selected from LISTS A, B, C, D, E and F. In illustrative examples of this type, the methods comprise detecting the level of at least one IRC marker expression product from LIST A and the level of at least one other IRC marker expression product from LIST B and the level of at least one other IRC marker expression product from LIST C. In other illustrative examples, the methods comprise detecting the level of at least one IRC marker expression product from LIST A and the level of at least one other IRC marker expression product from LIST B and the level of at least one other IRC marker expression product from LIST D. In still other illustrative examples, the methods comprise detecting the level of at least one IRC marker expression product from LIST A and the level of at least one other IRC marker expression product from LIST B and the level of at least one other IRC marker expression product from LIST E. In still other illustrative examples, the methods comprise detecting the level of at least one IRC marker expression product from LIST A and the level of at least one other IRC marker expression product from LIST B and the level of at least one other IRC marker expression product from LIST F. In other illustrative examples, the methods comprise detecting the level of at least one IRC marker expression product from LIST B and the level of at least one other IRC marker expression product from LIST C and the level of at least one other IRC marker expression product from LIST D. In still other illustrative examples, the methods comprise detecting the level of at least one IRC marker expression product from LIST B and the level of at least one other IRC marker expression product from LIST C and the level of at least one other IRC marker expression product from LIST E. In still other illustrative examples, the methods comprise detecting the level of at least one IRC marker expression product from LIST B and the level of at least one other IRC marker expression product from LIST C and the level of at least one other IRC marker expression product from LIST F. In other illustrative examples, the methods comprise detecting the level of at least one IRC marker expression product from LIST C and the level of at least one other IRC marker expression product from LIST D and the level of at least one other IRC marker expression product from LIST E. In still other illustrative examples, the methods comprise detecting the level of at least one IRC marker expression product from LIST C and the level of at least one other IRC marker expression product from LIST D and the level of at least one other IRC marker expression product from LIST F. In other illustrative examples, the methods comprise detecting the level of at least one IRC marker expression product from LIST D and the level of at least one other IRC marker expression product from LIST E and the level of at least one other IRC marker expression product from LIST F.

In still other embodiments, the methods comprise detecting the level of at least one IRC marker expression product from each of four lists selected from LISTS A, B, C, D, E and F. In illustrative examples of this type, the methods comprise detecting the level of at least one IRC marker expression product from LIST A and the level of at least one other IRC marker expression product from LIST B and the level of at least one other IRC marker expression product from LIST C and the level of at least one other IRC marker expression product from LIST D. In other illustrative examples, the methods comprise detecting the level of at least one IRC marker expression product from LIST A and the level of at least one other IRC marker expression product from LIST B and the level of at least one other IRC marker expression product from LIST C and the level of at least one other IRC marker expression product from LIST E. In other illustrative examples, the methods comprise detecting the level of at least one IRC marker expression product from LIST A and the level of at least one other IRC marker expression product from LIST B and the level of at least one other IRC marker expression product from LIST C and the level of at least one other IRC marker expression product from LIST F. In other illustrative examples, the methods comprise detecting the level of at least one IRC marker expression product from LIST A and the level of at least one other IRC marker expression product from LIST C and the level of at least one other IRC marker expression product from LIST D and the level of at least one other IRC marker expression product from LIST E. In other illustrative examples, the methods comprise detecting the level of at least one IRC marker expression product from LIST A and the level of at least one other IRC marker expression product from LIST C and the level of at least one other IRC marker expression product from LIST D and the level of at least one other IRC marker expression product from LIST F. In other illustrative examples, the methods comprise detecting the level of at least one IRC marker expression product from LIST A and the level of at least one other IRC marker expression product from LIST D and the level of at least one other IRC marker expression product from LIST E and the level of at least one other IRC marker expression product from LIST F. In still other illustrative examples, the methods comprise detecting the level of at least one IRC marker expression product from LIST B and the level of at least one other IRC marker expression product from LIST C and the level of at least one other IRC marker expression product from LIST D and the level of at least one other IRC marker expression product from LIST E. In still other illustrative examples, the methods comprise detecting the level of at least one IRC marker expression product from LIST B and the level of at least one other IRC marker expression product from LIST C and the level of at least one other IRC marker expression product from LIST D and the level of at least one other IRC marker expression product from LIST F. In other illustrative examples, the methods comprise detecting the level of at least one IRC marker expression product from LIST C and the level of at least one other IRC marker expression product from LIST D and the level of at least one other IRC marker expression product from LIST E and the level of at least one other IRC marker expression product from LIST F. In other illustrative examples, the methods comprise detecting the level of at least one IRC marker expression product from LIST A and the level of at least one other IRC marker expression product from LIST B and the level of at least one other IRC marker expression product from LIST D and the level of at least one other IRC marker expression product from LIST E.

In still other embodiments, the methods comprise detecting the level of at least one IRC marker expression product from each of five lists selected from LISTS A, B, C, D, E and F. In illustrative examples of this type, the methods comprise detecting the level of at least one IRC marker expression product from LIST A and the level of at least one other IRC marker expression product from LIST B and the level of at least one other IRC marker expression product from LIST C and the level of at least one other IRC marker expression product from LIST D and the level of at least one other IRC marker expression product from LIST E. In other illustrative example's, the methods comprise detecting the level of at least one IRC marker expression product from LIST A and the level of at least one other IRC marker expression product from LIST B and the level of at least one other IRC marker expression product from LIST C and the level of at least one other IRC marker expression product from LIST D and the level of at least one other IRC marker expression product from LIST F. In other illustrative examples, the methods comprise detecting the level of at least one IRC marker expression product from LIST A and the level of at least one other IRC marker expression product from LIST C and the level of at least one other IRC marker expression product from LIST D and the level of at least one other IRC marker expression product from LIST E and the level of at least one other IRC marker expression product from LIST F. In other illustrative examples, the methods comprise detecting the level of at least one IRC marker expression product from LIST B and the level of at least one other IRC marker expression product from LIST C and the level of at least one other IRC marker expression product from LIST D and the level of at least one other IRC marker expression product from LIST E and the level of at least one other IRC marker expression product from LIST F. In other illustrative examples, the methods comprise detecting the level of at least one IRC marker expression product from LIST B and the level of at least one other IRC marker expression product from LIST D and the level of at least one other IRC marker expression product from LIST E and the level of at least one other IRC marker expression product from LIST F and the level of at least one other IRC marker expression product from LIST A.

In still other embodiments, the methods comprise detecting the level of at least one IRC marker expression product from each of LISTS A, B, C, D, E and F.

In some embodiments, the methods further comprise diagnosing the absence of sepsis, inSIRS or post surgical inflammation when the measured level or functional activity of the or each IRC expression product is the same as or similar to the measured level or functional activity of the or each corresponding expression product when the control subject is a normal subject. In these embodiments, the measured level or functional activity of an individual IRC expression product varies from the measured level or functional activity of an individual corresponding expression product by no more than about 20%, 18%, 16%, 14%, 12%, 10%, 9%, 8%, 7%, 6%, 5%, 4%, 3%, 2%, 1% or 0.1%, which is hereafter referred to as “normal expression.”

In certain embodiments, a panel of IRC marker expression products is selected to distinguish sepsis from inSIRS, sepsis from post-surgical inflammation, sepsis from normal, inSIRS from post-surgical inflammation, inSIRS from normal or post-surgical from normal with at least about 70%, 80%, 85%, 90% or 95% sensitivity, suitably in combination with at least about 70% 80%, 85%, 90% or 95% specificity. In some embodiments, both the sensitivity and specificity are at least about 75%, 80%, 85%, 90% or 95%.

Advantageously, the biological sample comprises blood, especially peripheral blood, which suitably includes leukocytes. Suitably, the expression product is selected from a RNA molecule or a polypeptide. In some embodiments, the expression product is the same as the corresponding expression product. In other embodiments, the expression product is a variant (e.g., an allelic variant) of the corresponding expression product.

In certain embodiments, the expression product or corresponding expression product is a target RNA (e.g., mRNA) or a DNA copy of the target RNA whose level is measured using at least one nucleic acid probe that hybridists under at least low, medium, or high stringency conditions to the target RNA or to the DNA copy, wherein the nucleic acid probe comprises at least 15 contiguous nucleotides of an IRC marker polynucleotide. In these embodiments, the measured level or abundance of the target RNA or its DNA copy is normalized to the level or abundance of a reference RNA or a DNA copy of the reference RNA that is present in the same sample. Suitably, the nucleic acid probe is immobilized on a solid or semi-solid support. In illustrative examples of this type, the nucleic acid probe forms part of a spatial array of nucleic acid probes. In some embodiments, the level of nucleic acid probe that is bound to the target RNA or to the DNA copy is measured by hybridization (e.g., using a nucleic acid array). In other embodiments, the level of nucleic acid probe that is bound to the target RNA or to the DNA copy is measured by nucleic acid amplification (e.g., using a polymerase chain reaction (PCR)). In still other embodiments, the level of nucleic acid probe that is bound to the target RNA or to the DNA copy is measured by nuclease protection assay.

In other embodiments, the expression product or corresponding expression product is a target polypeptide whose level is measured using at least one antigen-binding molecule that is immuno-interactive with the target polypeptide. In these embodiments, the measured level of the target polypeptide is normalized to the level of a reference polypeptide that is present in the same sample. Suitably, the antigen-binding molecule is immobilized on a solid or semi-solid support. In illustrative examples of this type, the antigen-binding molecule forms part of a spatial array of antigen-binding molecule. In some embodiments, the level of antigen-binding molecule that is bound to the target polypeptide is measured by immunoassay (e.g., using an ELISA).

In still other embodiments, the expression product or corresponding expression product is a target polypeptide whose level is measured using at least one substrate for the target polypeptide with which it reacts to produce a reaction product. In these embodiments, the measured functional activity of the target polypeptide is normalized to the functional activity of a reference polypeptide that is present in the same sample.

In some embodiments, a system is used to perform the diagnostic methods as broadly described above, which suitably comprises at least one end station coupled to a base station. The base station is suitably caused (a) to receive subject data from the end station via a communications network, wherein the subject data represents parameter values corresponding to the measured or normalized level or functional activity of at least one expression product in the biological sample, and (b) to compare the subject data with predetermined data representing the measured or normalized level or functional activity of at least one corresponding expression product in the reference sample to thereby determine any difference in the level or functional activity of the expression product in the biological sample as compared to the level or functional activity of the corresponding expression product in the reference sample. Desirably, the base station is further caused to provide a diagnosis for the presence, absence or degree of post-surgical inflammation, inSIRS or sepsis. In these embodiments, the base station may be further caused to transfer an indication of the diagnosis to the end station via the communications network.

In another aspect, the invention contemplates use of the methods broadly described above in monitoring, treating or managing post-surgical inflammation or conditions that can lead to sepsis or inSIRS, illustrative examples of which include retained placenta, meningitis, endometriosis, shock, toxic shock (i.e., a sequelae to tampon use), gastroenteritis, appendicitis, ulcerative colitis, Crohn's disease, inflammatory bowel disease, acid gut syndrome, liver failure and cirrhosis, failure of colostrum transfer in neonates, ischemia (in any organ), bacteremia, infections within body cavities such as the peritoneal, pericardial, thecal, and pleural cavities, burns, severe wounds, excessive exercise or stress, hemodialysis, conditions involving intolerable pain (e.g., pancreatitis, kidney stones), surgical operations, and non-healing lesions. For these applications, the diagnostic methods of the invention are typically used at a frequency that is effective to monitor the early development of sepsis, inSIRS or post-surgical inflammation to thereby enable early therapeutic intervention and treatment of those conditions. In illustrative examples, the diagnostic methods are used at least at 1, 2, 3, 4, 5, 6, 7, 8, 9, 10, 11, 12, 13, 14, 15, 16, 17, 18, 19, 20, 21, 22, 23 or 24 hour intervals or at least 1, 2, 3, 4, 5 or 6 day intervals, or at least weekly, fortnightly or monthly.

Thus, in a related aspect, the present invention provides methods for treating, preventing or inhibiting the development of at least one condition selected from sepsis, inSIRS or post-surgical inflammation in a subject. These methods generally comprise:
comparing the level of at least one IRC expression product of a multi-transcript-producing gene in the subject to the level of a corresponding IRC marker expression product in at least one control subject selected from: a post-surgical inflammation-positive subject, an inSIRS positive subject, and a sepsis-positive subject, wherein a difference between the level of the at least one IRC marker expression product and the level of the corresponding IRC marker expression product indicates whether the subject has, or is at risk of developing, one of the conditions, wherein the at least one IRC marker expression product is predetermined as being differentially expressed between at least two of the conditions and wherein at least one other expression product from the multi-transcript producing gene is predetermined as being not so differentially expressed; and
administering to the subject, on the basis that the subject tests positive for sepsis, an effective amount of an agent that treats or ameliorates the symptoms or reverses or inhibits the development of sepsis, or
administering to the subject, on the basis that the subject tests positive for inSIRS, an effective amount of an agent that treats or ameliorates the symptoms or reverses or inhibits the development of inSIRS; or
administering to the subject, on the basis that the subject tests positive for post-surgical inflammation, an effective amount of an agent that treats or ameliorates the symptoms or reverses or inhibits the development of post-surgical inflammation.

Representative examples of sepsis treatments or agents include but are not limited to, antibiotics, intravenous fluids, vasoactives, palliative support for damaged or distressed organs (e.g. oxygen for respiratory distress, fluids for hypovolemia) and close monitoring of vital organs.

Non-limiting examples of such inSIRS treatments or agents include but are not limited to, antibiotics, steroids, intravenous fluids, glucocorticoids, vasoactives, palliative support for damaged or distressed organs (e.g. oxygen for respiratory distress, fluids for hypovolemia) and close monitoring of vital organs.

Illustrative examples of such post-surgical inflammation treatments or agents include but are not limited to, antibiotics, intravenous fluids, anti-inflammatory agents and immunomodulatory agents.

Still another aspect of the present invention provides the use of at least one IRC marker polynucleotide as broadly described above, or at least one IRC marker polypeptide as broadly described above, or at least one probe comprising or consisting essentially of a nucleic acid sequence which corresponds or is complementary to at least a portion of a nucleotide sequence encoding a IRC marker polypeptide as broadly described above, or the use of at least one antigen-binding molecule that is immuno-interactive with a IRC marker polypeptide as broadly described above, in the manufacture of a kit for assessing or diagnosing the presence or risk of development of, or distinguishing between, sepsis, inSIRS and post-surgical inflammation.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

The term “differential expression,” as used herein to describe the expression of an IRC expression product of a multi-transcript-producing gene, refers to the overexpression (up-regulation) or underexpression (down-regulation) of the IRC marker expression product (e.g., transcript or polypeptide) relative to the level of expression of a corresponding IRC marker expression product in a control subject as defined herein, and encompasses a higher or lower level of a IRC marker expression product (e.g., transcript or polypeptide) in a tissue sample or body fluid relative to a reference sample. In certain embodiments, an IRC marker expression product is differentially expressed if the level of the IRC marker expression product in a biological sample obtained from a test subject is at least 110%, 120%, 130%, 140%, 150%, 160%, 170%, 180%, 190%, 200%, 300%, 400%, 500%, 600%, 700%, 800%, 900% or 1000%, or no more than about 95%, 90%, 80%, 70%, 60%, 50%, 40%, 30%, 20%, 10%, 5%, 4%, 3%, 2%, 1%, 0.5%, 0.1%, 0.01%, 0.001% or 0.0001% of the level of expression of a corresponding IRC marker gene expression product in a reference sample obtained from a control subject as defined herein.

By “about” is meant a measurement, quantity, level, value, number, frequency, percentage, dimension, size, amount, weight or length that varies by as much as 10, 9, 8, 7, 6, 5, 4, 3, 2 or 1% to a reference measurement, quantity, level, value, number, frequency, percentage, dimension, size, amount, weight or length.

The term “amplicon” refers to a target sequence for amplification, and/or the amplification products of a target sequence for amplification. In certain other embodiments an “amplicon” may include the sequence of probes or primers used in amplification.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

As used herein, the term “binds specifically,” “specifically immuno-interactive” and the like when referring to an antigen-binding molecule refers to a binding reaction which is determinative of the presence of an antigen in the presence of a heterogeneous population of proteins and other biologics. Thus, under designated immunoassay conditions, the specified antigen-binding molecules bind to a particular antigen and do not bind in a significant amount to other proteins or antigens present in the sample. Specific binding to an antigen under such conditions may require an antigen-binding molecule that is selected for its specificity for a particular antigen. For example, antigen-binding molecules can be raised to a selected protein antigen, which bind to that antigen but not to other proteins present in a sample. A variety of immunoassay formats may be used to select antigen-binding molecules specifically immuno-interactive with a particular protein. For example, solid-phase ELISA immunoassays are routinely used to select monoclonal antibodies specifically immuno-interactive with a protein. See Harlow and Lane (1988) Antibodies, A Laboratory Manual, Cold Spring Harbor Publications, New York, for a description of immunoassay formats and conditions that can be used to determine specific immunoreactivity.

The term “biological sample” as used herein refers to a sample that may be extracted, untreated, treated, diluted or concentrated from an animal. The biological sample may include a biological fluid such as whole blood, serum, plasma, saliva, urine, sweat, ascitic fluid, peritoneal fluid, synovial fluid, amniotic fluid, cerebrospinal fluid, tissue biopsy, and the like. In certain embodiments, the biological sample is blood, especially peripheral blood.

Throughout this specification, unless the context requires otherwise, the words “comprise,” “comprises” and “comprising” will be understood to imply the inclusion of a stated step or element or group of steps or elements but not the exclusion of any other step or element or group of steps or elements:

By “corresponds to” or “corresponding to” is meant a polynucleotide (a) having a nucleotide sequence that is substantially identical or complementary to all or a portion of a reference polynucleotide sequence or (b) encoding an amino acid sequence identical to an amino acid sequence in a peptide or protein. This phrase also includes within its scope a peptide or polypeptide having an amino acid sequence that is substantially identical to a sequence of amino acids in a reference peptide or protein.

By “effective amount”, in the context of treating or preventing a condition is meant the administration of that amount of active to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

The terms “expression” or “gene expression” refer to production of RNA message or translation of RNA message into proteins or polypeptides, or both. Detection of either types of gene expression in use of any of the methods described herein is encompassed by the present invention.

By “expression vector” is meant any autonomous genetic element capable of directing the transcription of a polynucleotide contained within the vector and suitably the synthesis of a peptide or polypeptide encoded by the polynucleotide. Such expression vectors are known to practitioners in the art.

As used herein, the term “functional activity” generally refers to the ability of a molecule (e.g., a transcript or polypeptide) to perform its designated function including a biological, enzymatic, or therapeutic function. In certain embodiments, the functional activity of a molecule corresponds to its specific activity as determined by any suitable assay known in the art.

The term “gene” as used herein refers to any and all discrete coding regions of the cell's genome, as well as associated non-coding and regulatory regions. The gene is also intended to mean the open reading frame encoding specific polypeptides, introns, and adjacent 5′ and 3′ non-coding nucleotide sequences involved in the regulation of expression. In this regard, the gene may further comprise control signals such as promoters, enhancers, termination and/or polyadenylation signals that are naturally associated with a given gene, or heterologous control signals. The DNA sequences may be cDNA or genomic DNA or a fragment thereof. The gene may be introduced into an appropriate vector for extrachromosomal maintenance or for integration into the host.

By “high density polynucleotide arrays” and the like is meant those arrays that contain at least 400 different features per cm².

The phrase “high discrimination hybridization conditions” refers to hybridization conditions in which single base mismatch may be determined.

By “housekeeping gene” is meant a gene that is expressed in virtually all cells since it is fundamental to the any cell's functions (e.g., essential proteins and RNA molecules).

“Hybridization” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA, U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently.

The phrase “hybridizing specifically to” and the like refer to the binding, duplexing, or hybridizing of a molecule only to a particular nucleotide sequence under stringent conditions when that sequence is present in a complex mixture (e.g., total cellular) DNA or RNA.

Reference herein to “immuno-interactive” includes reference to any interaction, reaction, or other form of association between molecules and in particular where one of the molecules is, or mimics, a component of the immune system.

By “isolated” is meant material that is substantially or essentially free from components that normally accompany it in its native state. For example, an “isolated polynucleotide,” as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell, i.e., it is not associated with in vivo substances.

As used herein, a “naturally-occurring” nucleic acid molecule refers to a RNA or DNA molecule having a nucleotide sequence that occurs in nature. For example a naturally-occurring nucleic acid molecule can encode a protein that occurs in nature.

By “obtained” is meant to come into possession. Biological or reference samples so obtained include, for example, nucleic acid extracts or polypeptide extracts isolated or derived from a particular source. For instance, the extract may be isolated directly from a biological fluid or tissue of a subject.

The term “oligonucleotide” as used herein refers to a polymer composed of a multiplicity of nucleotide residues (deoxyribonucleotides or ribonucleotides, or related structural variants or synthetic analogues thereof, including nucleotides with modified or substituted sugar groups and the like) linked via phosphodiester bonds (or related structural variants or synthetic analogues thereof). Thus, while the term “oligonucleotide” typically refers to a nucleotide polymer in which the nucleotide residues and linkages between them are naturally-occurring, it will be understood that the term also includes within its scope various analogues including, but not restricted to, peptide nucleic acids (PNAs), phosphorothioate, phosphorodithioate, phosphoroselenoate, phosphorodiselenoate, phosphoroanilothioate, phosphoraniladate, phosphoroamidate, methyl phosphonates, 2-O-methyl ribonucleic acids, and the like. The exact size of the molecule can vary depending on the particular application. Oligonucleotides are a polynucleotide subset with 200 bases or fewer in length. Preferably, oligonucleotides are 10 to 60 bases in length and most preferably 12, 13, 14, 15, 16, 17, 18, 19, or 20 to 40 bases in length. Oligonucleotides are usually single stranded, e.g., for probes; although oligonucleotides may be double stranded, e.g., for use in the construction of a variant nucleic acid sequence. Oligonucleotides of the invention can be either sense or antisense oligonucleotides.

The term “oligonucleotide array” refers to a substrate having oligonucleotide probes with different known sequences deposited at discrete known locations associated with its surface. For example, the substrate can be in the form of a two dimensional substrate as described in U.S. Pat. No. 5,424,186. Such substrate may be used to synthesize two-dimensional spatially addressed oligonucleotide (matrix) arrays. Alternatively, the substrate may be characterized in that it forms a tubular array in which a two dimensional planar sheet is rolled into a three-dimensional tubular configuration. The substrate may also be in the form of a microsphere or bead connected to the surface of an optic fiber as, for example, disclosed by Chee et al. in WO 00/39587. Oligonucleotide arrays have at least two different features and a density of at least 400 features per cm². In certain embodiments, the arrays can have a density of about 500, at least one thousand, at least 10 thousand, at least 100 thousand, at least one million or at least 10 million features per cm². For example, the substrate may be silicon or glass and can have the thickness of a glass microscope slide or a glass cover slip, or may be composed of other synthetic polymers. Substrates that are transparent to light are useful when the method of performing an assay on the substrate involves optical detection. The term also refers to a probe array and the substrate to which it is attached that form part of a wafer.

The term “operably connected” or “operably linked” as used herein means placing a structural gene under the regulatory control of a promoter, which then controls the transcription and optionally translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting; i.e. the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting; i.e., the genes from which it is derived.

The term “pathogen” is used herein in its broadest sense to refer to an organism or an infectious agent whose infection of cells of viable animal tissue elicits a disease response.

The term “polynucleotide” or “nucleic acid” as used herein designates mRNA, RNA, cRNA, cDNA or DNA. The term typically refers to a polymeric form of nucleotides of at least 10 bases in length, either ribonucleotides or deoxynucleotides or a modified form of either type of nucleotide. The term includes single and double stranded forms of DNA.

The terms “polynucleotide variant” and “variant” refer to polynucleotides displaying substantial sequence identity with a reference polynucleotide sequence or polynucleotides that hybridize with a reference sequence under stringent conditions that are defined hereinafter. These terms also encompass polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains a biological function or activity of the reference polynucleotide. The terms “polynucleotide variant” and “variant” also include naturally-occurring allelic variants.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally-occurring amino acid, such as a chemical analogue of a corresponding naturally-occurring amino acid, as well as to naturally-occurring amino acid polymers.

The term “polypeptide variant” refers to polypeptides which are distinguished from a reference polypeptide by the addition, deletion or substitution of at least one amino acid residue. In certain embodiments, one or more amino acid residues of a reference polypeptide are replaced by different amino acids. It is well understood in the art that some amino acids may be changed to others with broadly similar properties without changing the nature of the activity of the polypeptide (conservative substitutions) as described hereinafter.

As used herein, “post-surgical inflammation” refers to a condition arising due to an immune response to a stimulus relating to a surgical insult. Post-surgical inflammation can be local or systemic and is often characterized by swelling, fever, pain and/or redness. Inflammation involves the movement of fluid and cells (e.g., white blood cells or leukocytes, neutrophils, monocytes and T- and B-cells) into the affected area, site or tissue. Excessive, misdirected and/or inappropriate immune inflammatory responses resulting from surgery can lead to SIRS and to damage of normal, healthy body tissues.

By “primer” is meant an oligonucleotide which, when paired with a strand of DNA, is capable of initiating the synthesis of a primer extension product in the presence of a suitable polymerizing agent. The primer is preferably single-stranded for maximum efficiency in amplification but can alternatively be double-stranded. A primer must be sufficiently long to prime the synthesis of extension products in the presence of the polymerization agent. The length of the primer depends on many factors, including application, temperature to be employed, template reaction conditions, other reagents, and source of primers. For example, depending on the complexity of the target sequence, the primer may be at least about 5, 6, 7, 8, 9, 10, 11, 12, 13, 14, 15, 16, 17, 18, 19, 20, 21, 22, 23, 24, 25, 26, 27, 28, 29, 30, 35, 40, 50, 75, 100, 150, 200, 300, 400, 500, to one base shorter in length than the template sequence at the 3′ end of the primer to allow extension of a nucleic acid chain, though the 5′ end of the primer may extend in length beyond the 3′ end of the template sequence. In certain embodiments, primers can be large polynucleotides, such as from about 35 nucleotides to several kilobases or more. Primers can be selected to be “substantially complementary” to the sequence on the template to which it is designed to hybridize and serve as a site for the initiation of synthesis. By “substantially complementary”, it is meant that the primer is sufficiently complementary to hybridize with a target polynucleotide. Desirably, the primer contains no mismatches with the template to which it is designed to hybridize but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridize therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another polynucleotide, often called the “target polynucleotide”, through complementary base pairing. Probes can bind target polynucleotides lacking complete sequence complementarity with the probe, depending on the stringency of the hybridization conditions. Probes can be labeled directly or indirectly and include primers within their scope.

The term “recombinant polynucleotide” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably linked to the nucleotide sequence.

By “recombinant polypeptide” is meant a polypeptide made using recombinant techniques, i.e., through the expression of a recombinant or synthetic polynucleotide.

By “regulatory element” or “regulatory sequence” is meant nucleic acid sequences (e.g., DNA) necessary for expression of an operably linked coding sequence in a particular host cell. The regulatory sequences that are suitable for prokaryotic cells for example, include a promoter, and optionally a cis-acting sequence such as an operator sequence and a ribosome binding site. Control sequences that are suitable for eukaryotic cells include promoters, polyadenylation signals, transcriptional enhancers, translational enhancers, leader or trailing sequences that modulate mRNA stability, as well as targeting sequences that target a product encoded by a transcribed polynucleotide to an intracellular compartment within a cell or to the extracellular environment.

As used herein, “sepsis” is defined as SIRS with a presumed or confirmed systemic infectious process. Confirmation of infectious process can be determined using microbiological culture or isolation of the infectious agent. From an immunological perspective, sepsis may be seen as a systemic response to systemic live microorganisms or systemic infection.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, H is, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, Calif., USA) using standard defaults as used in the reference manual accompanying the software.

“Similarity” refers to the percentage number of amino acids that are identical or constitute conservative substitutions as defined in Table A infra. Similarity may be determined using sequence comparison programs such as GAP (Deveraux et al. 1984, Nucleic Acids Research 12, 387-395). In this way, sequences of a similar or substantially different length to those cited herein might be compared by insertion of gaps into the alignment, such gaps being determined, for example, by the comparison algorithm used by GAP.

Terms used to describe sequence relationships between two or more polynucleotides or polypeptides include “reference sequence,” “comparison window,” “sequence identity,” “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

The terms “subject” or “individual” or “patient”, used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, primates, avians, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes). A preferred subject is an equine animal in need of treatment or prophylaxis of sepsis. However, it will be understood that the aforementioned terms do not imply that symptoms are present.

The phrase “substantially similar affinities” refers herein to target sequences having similar strengths of detectable hybridization to their complementary or substantially complementary oligonucleotide probes under a chosen set of stringent conditions.

“Systemic Inflammatory Response Syndrome (SIRS),” as used herein, refers to a clinical response arising from a non-specific insult with two or more of the following measurable clinical characteristics; a body temperature greater than 38° C. or less than 36° C., a heart rate greater than 90 beats per minute, a respiratory rate greater than 20 per minute, a white blood cell count (total leukocytes) greater than 12,000 per mm³ or less than 4,000 per mm³, or a band neutrophil percentage greater than 10%. From an immunological perspective, it may be seen as representing a systemic response to insult (e.g., major surgery) or systemic inflammation. As used herein, therefore, “infection-negative SIRS (inSIRS)” includes the clinical response noted above but in the absence of a systemic infectious process.

The term “template” as used herein refers to a nucleic acid that is used in the creation of a complementary nucleic acid strand to the “template” strand. The template may be either RNA and/or DNA, and the complementary strand may also be RNA and/or DNA. In certain embodiments, the complementary strand may comprise all or part of the complementary sequence to the “template,” and/or may include mutations so that it is not an exact, complementary strand to the “template”. Strands that are not exactly complementary to the template strand may hybridize specifically to the template strand in detection assays described here, as well as other assays known in the art, and such complementary strands that can be used in detection assays are part of the invention.

The term “transformation” means alteration of the genotype of an organism, for example a bacterium, yeast, mammal, avian, reptile, fish or plant, by the introduction of a foreign or endogenous nucleic acid.

The term “treat” is meant to include both therapeutic and prophylactic treatment.

By “vector” is meant a polynucleotide molecule, suitably a DNA molecule derived, for example, from a plasmid, bacteriophage, yeast, virus, mammal, avian, reptile or fish into which a polynucleotide can be inserted or cloned. A vector preferably contains one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector can also include a selection marker such as an antibiotic resistance gene that can be used for selection of suitable transformants. Examples of such resistance genes are known to those of skill in the art.

The terms “wild-type” and “normal” are used interchangeably to refer to the phenotype that is characteristic of most of the members of the species occurring naturally and contrast for example with the phenotype of a mutant.

### 2. Abbreviations

The following abbreviations are used throughout the application:

nt=nucleotide

nts=nucleotides

aa=amino acid(s)

kb=kilobase(s) or kilobase pair(s)

kDa=kilodalton(s)

d=day

h=hour

s=seconds

### 3. Markers of Sepsis, inSIRS and Post-Surgical Inflammation and Uses Therefor

The present invention is predicated in part on the identification of 235 genes that show evidence of splice variation in which only particular splice variants of individual genes differ in expression between sepsis-positive patients, inSIRS-positive patients and post-surgical patients. Of these 235 multi-transcript-producing genes, only a limited number (57) were found to express specific splice variants, which comprise “condition-separating exons” and which are useful as classifiers to distinguish between these patient groups. These multi-transcript-producing genes are listed in Table 1.

Thus, in accordance with the present invention, specific splice variants of the above multi-transcript-producing genes and their polypeptide products provide a means for separating sepsis, inSIRS and post-surgical inflammation, allowing for qualitative or quantitative grading of inflammatory response as if there were a “continuum” of severity of inflammatory response from post-surgical inflammation through to sepsis. These markers are thus designated herein “inflammatory response continuum” or “IRC” marker expression products, which are listed in Table 2, 3 and 4.

Accordingly, the IRC marker expression products of the present invention are useful in methods for diagnosis, detection of host response, determining degree of host response, monitoring, treatment or management of, or distinguishing between, infection-negative systemic inflammatory response syndrome (inSIRS) and sepsis as well as post-surgical inflammation in mammals. More particularly, the present invention relates to the use of specific expression products from a multi-transcript-producing gene for distinguishing between inSIRS and sepsis and post-surgical inflammation.

In specific embodiments, the IRC markers are in the form of RNA molecules of specified sequences, or polypeptides transcribed from these RNA molecules in cells, especially in blood cells, and more especially in peripheral blood cells, of subjects with or susceptible to sepsis/inSIRS/post-surgical inflammation, are disclosed. These markers are indicators of sepsis/inSIRS/post-surgical inflammation and, when differentially expressed as compared to their expression in control subjects selected from sepsis-positive subjects, inSIRS-positive subjects, post-surgical inflammation positive subjects and normal subjects or subjects that do not have any of these conditions, they distinguish between, and are diagnostic for the presence or absence of, those conditions in tested subjects. Such markers provide considerable advantages over the prior art in this field. In certain advantageous embodiments where leukocytes (e.g., peripheral blood cells) are used for the analysis, it is possible to diagnose sepsis before serum antibodies to endotoxin, or endotoxemia-causing agents are detected.

It will be apparent that the nucleic acid sequences disclosed herein (also referred to herein as “IRC marker polynucleotides”) will find utility in a variety of applications in detection, diagnosis, prognosis and treatment of sepsis, inSIRS and post-surgical inflammation. Examples of such applications within the scope of the present disclosure include amplification of IRC marker polynucleotides using specific primers, detection of IRC marker polynucleotides by hybridization with oligonucleotide probes, incorporation of isolated nucleic acids into vectors, expression of vector-incorporated nucleic acids as RNA and protein, and development of immunological/detection/diagnostic/prognostic reagents corresponding to marker encoded products.

The identified IRC marker polynucleotides may in turn be used to design specific oligonucleotide probes and primers. Such probes and primers may be of any length that would specifically hybridize to the identified IRC marker polynucleotides and may be at least about 10, 11, 12, 13, 14, 15, 16, 17, 18, 19, 20, 21, 22, 23, 24, 25, 26, 27, 28, 29, 30, 35, 40, 50, 75, 100, 150, 200, 300, 400, 500 nucleotides in length and in the case of probes, up to the full-length of the sequences of one or more of condition-separating exons contained in a IRC marker polynucleotide or up to the full-length of an IRC marker polynucleotide as identified herein. Probes may, also include additional sequence at their 5′ and/or 3′ ends so that they extent beyond the target sequence with which they hybridize.

When used in combination with nucleic acid amplification procedures, these probes and primers enable the rapid analysis of biological samples (e.g., peripheral blood samples) for detecting or quantifying IRC marker polynucleotides (e.g., transcripts). Such procedures include any method or technique known in the art or described herein for duplicating or increasing the number of copies or amount of a target nucleic acid or its complement.

One of ordinary skill in the art could select segments from the identified IRC marker polynucleotides and their encoded polypeptide products (also referred to herein as “IRC marker polypeptides”) for use in the different detection, diagnostic, or prognostic methods, vector constructs, antigen-binding molecule production, kit, and/or any of the embodiments described herein as part of the present invention. Representative sequences that are desirable for use in the invention are those set forth in SEQ ID NO: 1-88 (see Tables 2, 3 and 4).

### 4. Nucleic Acid Molecules of the Invention

As described in the Examples and in Tables 1-4, the present disclosure provides IRC marker polynucleotides comprising condition-separating exons from 57 multi-transcript-producing genes selected from ANKDD1A, GABRR2, OTX1, PANX2, RHBDF2, SLAMF7, AMBRA1, CES2, CLPB, HIPK2, C1ORF91, NDST1, SLC36A1, ADAM19, CUL7, TG, PDCD1LG2, GRINL1A, MGRN1, SNTB2, CDK5R1, GAA, KATNAL2, CEACAM4, ZNF335, ASPHD2, ACRC, BTNL8, MOV10, MED12L, KLHL6, PDLIM5, GALNT10, SCRN1, VOPP1, FKBP9, KIF27, PIWIL4, TEP1, GCH1, PRR11, CDH2, PPM1N, RRAS, DDOST, APH1A, TTL, TEX261, COQ2, FCHSD1, BAK1, SLC25A25, RELT, ACP2, TBC1D2B, FANCA or SLC39A11. Representative IRC marker polynucleotides have been identified by exon array analysis of blood obtained from patients with clinical evidence of sepsis or inSIRS or post-surgical inflammation and these are set forth in SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513 or 515. These sequences, which are presented in Tables 2-4, are diagnostic for the presence or absence of sepsis or inSIRS or post-surgical inflammation.

In accordance with the present invention, the sequences of isolated nucleic acids disclosed herein find utility inter alia as hybridization probes or amplification primers. In certain embodiments, these probes and primers represent oligonucleotides, which are of sufficient length to provide specific hybridization to a RNA or DNA sample extracted from the biological sample. The sequences typically will be about 10-20 nucleotides, but may be longer. Longer sequences, e.g., of about 30, 40, 50, 100, 500 and even up to the full-length of condition-separating exons or of the IRC marker polynucleotides, are desirable for certain embodiments.

Nucleic acid molecules having contiguous stretches of about 10, 15, 17, 20, 30, 40, 50, 60, 75 or 100 or 500 nucleotides of a sequence set forth in any one of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513 or 515 are contemplated. Molecules that are complementary to the above mentioned sequences and that bind to these sequences under high stringency conditions are also contemplated. These probes are useful in a variety of hybridization embodiments, such as Southern and northern blotting. In some cases, it is contemplated that probes may be used that hybridize to multiple target sequences without compromising their ability to effectively diagnose the presence or absence or distinguish between sepsis, inSIRS and post-surgical inflammation. In general, it is contemplated that the hybridization probes described herein are useful both as reagents in solution hybridization, as in PCR, for detection of expression of corresponding genes, as well as in embodiments employing a solid phase.

Various probes and primers may be designed around the disclosed nucleotide sequences. For example, in certain embodiments, the sequences used to design probes and primers may include repetitive stretches of adenine nucleotides (poly-A tails) normally attached at the ends of the RNA for the identified marker genes. In other embodiments, probes and primers may be specifically designed to not include these or other segments from the identified marker genes, as one of ordinary skilled in the art may deem certain segments more suitable for use in the detection methods disclosed. In any event, the choice of primer or probe sequences for a selected application is within the realm of the ordinary skilled practitioner. Illustrative primer/probe sequences for detection of IRC marker polynucleotides are presented in Table 5.

Primers may be provided in double-stranded or single-stranded form, although the single-stranded form is desirable. Probes, while perhaps capable of priming, are designed to bind to a target DNA or RNA and need not be used in an amplification process. In certain embodiments, the probes or primers are labeled with radioactive species ³²P, ¹⁴C, ³⁵S, ³H, or other label), with a fluorophore (e.g., rhodamine, fluorescein) or with a chemillumiscent label (e.g., luciferase).

The present invention provides substantially full-length cDNA sequences that are useful as markers of sepsis, inSIRS and post-surgical inflammation. It will be understood, however, that the present disclosure is not limited to these disclosed sequences and is intended particularly to encompass at least isolated nucleic acids that are hybridizable to nucleic acids comprising the disclosed sequences or that are variants of these nucleic acids. For example, a nucleic acid of partial sequence may be used to identify a structurally-related gene or the full-length genomic or cDNA clone from which it is derived. Methods for generating cDNA and genomic libraries which may be used as a target for the above-described probes are known in the art (see, for example, Sambrook et al., 1989, supra and Ausubel et al., 1994, supra). All such nucleic acids as well as the specific nucleic acid molecules disclosed herein are collectively referred to as “IRC marker polynucleotides.” Additionally, the present invention includes within its scope isolated or purified polypeptide products of IRC marker polynucleotides.

As such, the present invention encompasses isolated or substantially purified nucleic acid or protein compositions. An “isolated” or “purified” nucleic acid molecule or protein, or biologically active portion thereof, is substantially or essentially free from components that normally accompany or interact with the nucleic acid molecule or protein as found in its naturally occurring environment. Thus, an isolated or purified polynucleotide or polypeptide is substantially free of other cellular material, or culture medium when produced by recombinant techniques, or substantially free of chemical precursors or other chemicals when chemically synthesized. Suitably, an “isolated” polynucleotide is free of sequences (especially protein encoding sequences) that naturally flank the polynucleotide (i.e., sequences located at the 5′ and 3′ ends of the polynucleotide) in the genomic DNA of the organism from which the polynucleotide was derived. For example, in various embodiments, an isolated IRC marker polynucleotide can contain less than about 5 kb, 4 kb, 3 kb, 2 kb, 1 kb, 0.5 kb, or 0.1 kb of nucleotide sequences that naturally flank the polynucleotide in genomic DNA of the cell from which the polynucleotide was derived. A polypeptide that is substantially free of cellular material includes preparations of protein having less than about 30%, 20%, 10%, 5%, (by dry weight) of contaminating protein. When the IRC marker polypeptide is recombinantly produced, culture medium suitably represents less than about 30%, 20%, 10%, or 5% (by dry weight) of chemical precursors or non-protein-of-interest chemicals.

The invention also contemplates variants of the IRC marker polynucleotides. Nucleic acid variants can be naturally-occurring, such as allelic variants (same locus), homologues (different locus), and orthologues (different organism) or can be non naturally-occurring. Naturally occurring variants such as these can be identified with the use of well-known molecular biology techniques, as, for example, with polymerase chain reaction (PCR) and hybridization techniques as known in the art. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product). For nucleotide sequences, conservative variants include those sequences that, because of the degeneracy of the genetic code, encode the amino acid sequence of one of the IRC marker polypeptides of the invention. Variant nucleotide sequences also include synthetically derived nucleotide sequences, such as those generated, for example, by using site-directed mutagenesis but which still encode an IRC marker polypeptide of the invention. Generally, variants of a particular nucleotide sequence of the invention will have at least about 70%, 75%, 80%, 85%, desirably about 90%, 91%, 92%, 93%, 94% to 95% or more, and more suitably about 96%, 97%, 98%, 99% or more sequence identity to that particular nucleotide sequence as determined by sequence alignment programs described elsewhere herein using default parameters.

The IRC marker polynucleotides of the invention can be used to isolate corresponding sequences and alleles from other organisms, particularly other mammals. Methods are readily available in the art for the hybridization of nucleic acid sequences. Coding sequences from other organisms may be isolated according to well known techniques based on their sequence identity with the coding sequences set forth herein. In these techniques all or part of the known coding sequence is used as a probe which selectively hybridizes to other IRC marker polynucleotide coding sequences present in a population of cloned cDNA fragments (i.e., cDNA libraries) from a chosen organism. Accordingly, the present invention also contemplates polynucleotides that hybridize to the IRC marker polynucleotide sequences, or to their complements, under stringency conditions described below. As used herein, the term “hybridizes under low stringency, medium stringency, high stringency, or very high stringency conditions” describes conditions for hybridization and washing. Guidance for performing hybridization reactions can be found in Ausubel et al., (1998, supra), Sections 6.3.1-6.3.6. Aqueous and non-aqueous methods are described in that reference and either can be used. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at room temperature. One embodiment of low stringency conditions includes hybridization in 6× sodium chloride/sodium citrate (SSC) at about 45° C., followed by two washes in 0.2×SSC, 0.1% SDS at least at 50° C. (the temperature of the washes can be increased to 55° C. for low stringency conditions). Medium stringency conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridization at 65° C., and (i) 2×SSC, 0.1% SDS; or (ii) 0.5% BSA, 1 mM EDTA, 40 mM NaHPO₄ (pH 7.2), 5% SDS for washing at 60-65° C. One embodiment of medium stringency conditions includes hybridizing in 6×SSC at about 45° C., followed by one or more washes in 0.2×SSC, 0.1% SDS at 60° C. High stringency conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridization at 65° C., and (i) 0.2×SSC, 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridizing in 6×SSC at about 45° C., followed by one or more washes in 0.2×SSC, 0.1% SDS at 65° C.

In certain embodiments, an IRC marker polynucleotide of the invention is encoded by a polynucleotide that hybridizes to a disclosed nucleotide sequence (and suitably comprises a condition-separating exon as defined herein) under very high stringency conditions. One embodiment of very high stringency conditions includes hybridizing 0.5 M sodium phosphate, 7% SDS at 65° C., followed by one or more washes at 0.2×SSC, 1% SDS at 65° C.

Other stringency conditions are well known in the art and a skilled addressee will recognize that various factors can be manipulated to optimize the specificity of the hybridization. Optimization of the stringency of the final washes can serve to ensure a high degree of hybridization. For detailed examples, see Ausubel et al., supra at pages 2.10.1 to 2.10.16 and Sambrook et al. (1989, supra) at sections 1.101 to 1.104.

While stringent washes are typically carried out at temperatures from about 42° C. to 68° C., one skilled in the art will appreciate that other temperatures may be suitable for stringent conditions. Maximum hybridization rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel et al., supra at page 2.10.8). In general, the Tₘ of a perfectly matched duplex of DNA may be predicted as an approximation by the formula:

Tₘ=81.5+16.6(log₁₀ M)+0.41(% G+C)−0.63(% formamide)−(600/length)

wherein: M is the concentration of Na⁺, preferably in the range of 0.01 molar to 0.4 molar; % G+C is the sum of guanosine and cytosine bases as a percentage of the total number of bases, within the range between 30% and 75% G+C; % formamide is the percent formamide concentration by volume; length is the number of base pairs in the DNA duplex. The Tₘ of a duplex DNA decreases by approximately 1° C. with every increase of 1% in the number of randomly mismatched base pairs. Washing is generally carried out at Tₘ−15° C. for high stringency, or Tₘ−30° C. for moderate stringency.

In one example of a hybridization procedure, a membrane (e.g., a nitrocellulose membrane or a nylon membrane) containing immobilized DNA is hybridized overnight at 42° C. in a hybridization buffer (50% deionized formamide, 5×SSC, 5×Denhardt's solution (0.1% ficoll, 0.1% polyvinylpyrrolidone and 0.1% bovine serum albumin), 0.1% SDS and 200 mg/mL denatured salmon sperm DNA) containing labeled probe. The membrane is then subjected to two sequential medium stringency washes (i.e., 2×SSC, 0.1% SDS for 15 min at 45° C., followed by 2×SSC, 0.1% SDS for 15 min at 50° C.), followed by two sequential higher stringency washes (i.e., 0.2×SSC, 0.1% SDS for 12 min at 55° C. followed by 0.2×SSC and 0.1% SDS solution for 12 min at 65-68° C.

### 5. Polypeptides of the Invention

The present invention also contemplates the use of full-length polypeptides encoded by the IRC marker polynucleotides of the invention as well as their fragments, which are referred to collectively herein as “IRC marker polypeptides” for use as positive controls in the methods of the invention. Fragments of full-length IRC marker polypeptides include amino acid sequences encoded by condition-separating exons as defined herein and may comprise 6, 8, 10, 12, 14, 16, 18, 20, 25, 30, 40, 50, 60 amino acid residues in length. For example, fragments contemplated by the present invention are at least 6 and desirably at least 8 amino acid residues in length, which can elicit an immune response in an animal for the production of antigen-binding molecules that are immuno-interactive with an IRC marker polypeptide of the invention. Such antigen-binding molecules can be used to screen vertebrate animals, especially mammals, for structurally and/or functionally related IRC marker polypeptides: Fragments of a full-length IRC marker polypeptide include peptides comprising amino acid sequences sufficiently similar to or derived from the amino acid sequences of a (putative) full-length IRC marker polypeptide, for example, the amino acid sequences shown in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514 or 516, which include less amino acids than a full-length IRC marker polypeptide. A fragment of a full-length IRC marker polypeptide can be a polypeptide which is, for example, 5, 6, 7, 8, 9, 10, 11, 12, 13, 14, 15, 16, 17, 18, 19, 20, 21, 22, 23, 24, 25, 30, 40, 50, 60, 70, 80, 90, 100, 120, 150, 300, 400, 500, 600, 700, 800, 900 or 1000, or even at least about 2000 or 3000, or more amino acid residues in length.

The present invention also contemplates detecting variant IRC marker polypeptides, which comprise an amino acid sequence encoded by a condition-separating exon or variant thereof, in the methods of the invention. “Variant” polypeptides include proteins derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active, that is, they continue to possess the desired biological activity of the native protein. Such variants may result from, for example, genetic polymorphism or from human manipulation. Variants of an IRC marker polypeptide will have at least 40%, 50%, 60%, 70%, generally at least 75%, 80%, 85%, preferably about 90% to 95% or more, and more preferably about 98% or more sequence similarity with the amino acid sequence for a reference IRC polypeptide as determined by sequence alignment programs described elsewhere herein using default parameters. A variant of an IRC polypeptide of the invention may differ from that protein generally by as much 200, 100, 50 or 20 amino acid residues or suitably by as few as 1-15 amino acid residues, as few as 1-10, such as 6-10, as few as 5, as few as 4, 3, 2, or even 1 amino acid residue.

Variant IRC marker polypeptides may contain conservative amino acid substitutions at various locations along their sequence, as compared to a reference IRC marker amino add sequence. A “conservative amino acid substitution” is one in which the amino acid residue is replaced with an amino acid residue having a similar side chain. Families of amino acid residues having similar side chains have been defined in the art, which can be generally sub-classified as follows:

Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residues are charged at physiological pH and, therefore, include amino acids having acidic or basic side chains (i.e., glutamic acid, aspartic acid, arginine, lysine and histidine).

Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.

Neutral/polar: The residues are not charged at physiological pH, but the residue is not sufficiently repelled by aqueous solutions so that it would seek inner positions in the conformation of a peptide in which it is contained when the peptide is in aqueous medium. Amino acids having a neutral/polar side chain include asparagine, glutamine, cysteine, histidine, serine and threonine.

This description also characterizes certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices (e.g., PAM120 matrix and PAM250 matrix as disclosed for example by Dayhoff et al. (1978) A model of evolutionary change in proteins. Matrices for determining distance relationships In M. O. Dayhoff, (ed.), Atlas of protein sequence and structure, Vol. 5, pp. 345-358, National Biomedical Research Foundation, Washington D.C.; and by Gonnet et al., 1992, Science 256(5062): 144301445), however, include proline in the same group as glycine, serine, alanine and threonine. Accordingly, for the purposes of the present invention, proline is classified as a “small” amino acid.

The degree of attraction or repulsion required for classification as polar or nonpolar is arbitrary and, therefore, amino acids specifically contemplated by the invention have been classified as one or the other. Most amino acids not specifically named can be classified on the basis of known behavior.

Amino acid residues can be further sub-classified as cyclic or noncyclic, and aromatic or nonaromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small residues are, of course, always nonaromatic. Dependent on their structural properties, amino acid residues may fall in two or more classes. For the naturally-occurring protein amino acids, sub-classification according to the this scheme is presented in the Table 6.

Accordingly, the present invention also contemplates variants of the reference IRC marker polypeptide sequences or their fragments, wherein the variants are distinguished from the reference sequence by the addition, deletion, or substitution of one or more amino acid residues. In general, variants will display at least about 70, 75, 80, 85, 90, 91, 92, 93, 94, 95, 96, 97, 98, 99% similarity to a reference IRC marker polypeptide sequence as, for example, set forth in any one of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514 or 516. Desirably, variants will have at least 70, 75, 80, 85, 90, 91, 92, 93, 94, 95, 96, 97, 98, 99% sequence identity to a reference IRC marker polypeptide sequence as, for example, set forth in any one of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514 or 516. Moreover, sequences differing from the native or reference sequences by the addition, deletion, or substitution of 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 300, 500 or more amino acids but which comprise an amino acid sequence encoded by a condition-separating exon as defined herein, are contemplated. IRC marker polypeptides also include polypeptides that are encoded by polynucleotides that hybridize under stringency conditions as defined herein, especially high stringency conditions, to the IRC marker polynucleotide sequences of the invention, or to the non-coding strand thereof, as described above, which comprise condition-separating exons.

In some embodiments, variant polypeptides differ from an IRC marker sequence by at least one but by less than 50, 40, 30, 20, 15, 10, 8, 6, 5, 4, 3 or 2 amino acid residue(s). In other embodiments, variant polypeptides differ from the corresponding sequence in any one of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490; 492, 494, 496, 498, 500, 502, 504, 506, 508, 510, 512, 514 or 516 by at least 1% but less than 20%, 15%, 10% or 5% of the residues. (If this comparison requires alignment the sequences should be aligned for maximum similarity.

In other embodiments, a variant IRC polypeptide includes an amino acid sequence having at least about 50%, 55%, 60%, 65%, 70%, 75%, 80%, 85%, 90%, 91%, 92%, 93%, 94% 95%, 96%, 97%, 98% or more similarity to a corresponding sequence of an IRC marker polypeptide as, for example, set forth in any one of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514 or 516, and which comprise an amino acid sequence encoded by a condition-separating exon.

IRC marker polypeptides of the invention may be prepared by any suitable procedure known to those of skill in the art. For example, the polypeptides may be prepared by a procedure including the steps of: (a) preparing a chimeric construct comprising a nucleotide sequence that encodes at least a portion of an IRC marker polynucleotide and that is operably linked to a regulatory element; (b) introducing the chimeric construct into a host cell; (c) culturing the host cell to express the IRC marker polypeptide; and (d) isolating the IRC marker polypeptide from the host cell. In illustrative examples, the nucleotide sequence encodes at least a portion of the sequence set forth in any one of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514 or 516, or a variant thereof.

The chimeric construct is typically in the form of an expression vector, which is suitably selected from self-replicating extra-chromosomal vectors (e.g., plasmids) and vectors that integrate into a host genome.

The regulatory element will generally be appropriate for the host cell employed for expression of the IRC marker polynucleotide. Numerous types of expression vectors and regulatory elements are known in the art for a variety of host cells. Illustrative elements of this type include, but are not restricted to, promoter sequences (e.g., constitutive or inducible promoters which may be naturally occurring or combine elements of more than one promoter), leader or signal sequences, ribosomal binding sites, transcriptional start and stop sequences, translational start and termination sequences, and enhancer or activator sequences.

In some embodiments, the expression vector comprises a selectable marker gene to permit the selection of transformed host cells. Selectable marker genes are well known in the art and will vary with the host cell employed.

The expression vector may also include a fusion partner (typically provided by the expression vector) so that the IRC marker polypeptide is produced as a fusion polypeptide with the fusion partner.

The chimeric constructs of the invention are introduced into a host by any suitable means including “transduction” and “transfection”, which are art recognized as meaning the introduction of a nucleic acid, for example, an expression vector, into a recipient cell by nucleic acid-mediated gene transfer. “Transformation,” however, refers to a process in which a host's genotype is changed as a result of the cellular uptake of exogenous DNA or RNA, and, for example, the transformed cell comprises the expression system of the invention. There are many methods for introducing chimeric constructs into cells. Typically, the method employed will depend on the choice of host cell. Technology for introduction of chimeric constructs into host cells is well known to those of skill in the art. Four general classes of methods for delivering nucleic acid molecules into cells have been described: (1) chemical methods such as calcium phosphate precipitation, polyethylene glycol (PEG)-mediated precipitation and lipofection; (2) physical methods such as microinjection, electroporation, acceleration methods and vacuum infiltration; (3) vector based methods such as bacterial and viral vector-mediated transformation; and (4) receptor-mediated. Transformation techniques that fall within these and other classes are well known to workers in the art, and new techniques are continually becoming known. The particular choice of a transformation technology will be determined by its efficiency to transform certain host species as well as the experience and preference of the person practicing the invention with a particular methodology of choice. It will be apparent to the skilled person that the particular choice of a transformation system to introduce a chimeric construct into cells is not essential to or a limitation of the invention, provided it achieves an acceptable level of nucleic acid transfer.

Recombinant IRC marker polypeptides may be produced by culturing a host cell transformed with a chimeric construct. The conditions appropriate for expression of the IRC marker polynucleotide will vary with the choice of expression vector and the host cell and are easily ascertained by one skilled in the art through routine experimentation. Suitable host cells for expression may be prokaryotic or eukaryotic. An illustrative host cell for expression of a polypeptide of the invention is a bacterium. The bacterium used may be Escherichia coli. Alternatively, the host cell may be a yeast cell or an insect cell such as, for example, SF9 cells that may be utilized with a baculovirus expression system.

Recombinant IRC marker polypeptides or their fragments that comprise an amino acid sequence encoded by a condition-separating exon, as well as variants thereof, can be conveniently prepared using standard protocols as described for example in Sambrook, et al., (1989, supra), in particular Sections 16 and 17; Ausubel et al., (1994, supra), in particular Chapters 10 and 16; and Coligan et al., CURRENT PROTOCOLS IN PROTEIN SCIENCE (John Wiley & Sons, Inc. 1995-1997), in particular Chapters 1, 5 and 6. Alternatively, the IRC marker polypeptides may be synthesized by chemical synthesis, e.g., using solution synthesis or solid phase synthesis as described, for example, in Chapter 9 of Atherton and Shephard (supra) and in Roberge et al (1995, Science 269: 202).

### 6. Thresholds

In some embodiments, the methods comprise comparing the level or functional activity of individual expression products to one or more preselected or threshold levels or functional activities. Thresholds may be selected that provide an acceptable ability to predict diagnosis, prognostic risk, treatment success, etc. In illustrative examples, receiver operating characteristic (ROC) curves are calculated by plotting the value of a variable versus its relative frequency in two populations (called arbitrarily, for example, “sepsis” and “inSIRS,” “sepsis” and “post-surgical inflammation,” “sepsis” and “normal,” “inSIRS” and “post-surgical inflammation,” “inSIRS” and “normal,” “post-surgical inflammation” and “normal,” or simply “disease” and “normal” or “low risk” and “high risk”).

For any particular IRC marker expression product, a distribution of expression product levels or functional activities for subjects with and without a disease will likely overlap. Under such conditions, a test does not absolutely distinguish “disease” and “normal” with 100% accuracy, and the area of overlap indicates where the test cannot distinguish “disease” and “normal.” A threshold is selected, above which (or below which, depending on how an IRC marker expression product changes with the disease or prognosis) the test is considered to be “positive” and below which the test is considered to be “negative.” The area under the ROC curve is a measure of the probability that the perceived measurement will allow correct identification of a condition (see, e.g., Hanley et al., Radiology 143: 29-36 (1982). Alternatively, or in addition, thresholds may be established by obtaining an earlier marker gene expression product result from the same patient, to which later results may be compared. In these embodiments, the individual in effect acts as their own “control group.” In markers that increase with disease severity or prognostic risk, an increase over time in the same patient can indicate a worsening of disease or a failure of a treatment regimen, while a decrease over time can indicate remission of disease or success of a treatment regimen.

In certain embodiments, a panel of IRC marker expression products is selected to distinguish any pair of groups selected from “sepsis” and “inSIRS,” “sepsis” and “post-surgical inflammation,” “sepsis” and “normal,” “inSIRS” and “post-surgical inflammation,” “inSIRS” and “normal,” “post-surgical inflammation” and “normal,” “disease” and “normal” or “low risk” and “high risk” with at least about 70%, 80%, 85%, 90% or 95% sensitivity, suitably in combination with at least about 70% 80%, 85%, 90% or 95% specificity. In some embodiments, both the sensitivity and specificity are at least about 75%, 80%, 85%, 90% or 95%.

In some embodiments, a positive likelihood ratio, negative likelihood ratio, odds ratio, or hazard ratio is used as a measure of the ability of the methods of the present invention to predict disease, prognostic risk, or treatment outcome. In the case of a positive likelihood ratio, a value of 1 indicates that a positive result is equally likely among subjects in both the diseased group (e.g., one of sepsis, inSIRS or post-surgical inflammation) and control group (e.g., one of sepsis, inSIRS or post-surgical inflammation, which is other than the diseased group, or normal); a value greater than 1 indicates that a positive result is more likely in the diseased group; and a value less than 1 indicates that a positive result is more likely in the control group. In the case of a negative likelihood ratio, a value of 1 indicates that a negative result is equally likely among subjects in both groups; a value greater than 1 indicates that a negative result is more likely in the diseased group; and a value less than 1 indicates that a negative result is more likely in the control group. In certain embodiments, IRC markers and/or IRC marker panels are selected to exhibit a positive or negative likelihood ratio of at least about 1.5 or more or about 0.67 or less, at least about 2 or more or about 0.5 or less, at least about 5 or more or about 0.2 or less, at least about 10 or more or about 0.1 or less, or at least about 20 or more or about 0.05 or less.

In the case of an odds ratio, a value of 1 indicates that a positive result is equally likely among subjects in both the diseased and control groups; a value greater than 1 indicates that a positive result is more likely in the diseased group; and a value less than 1 indicates that a positive result is more likely in the control group. In certain embodiments, IRC markers and/or IRC marker panels are selected to exhibit an odds ratio of at least about 2 or more or about 0.5 or less, at least about 3 or more or about 0.33 or less, at least about 4 or more or about 0.25 or less, at least about 5 or more or about 0.2 or less, or at least about 10 or more or about 0.1 or less.

In the case of a hazard ratio, a value of 1 indicates that the relative risk is equal in both the diseased and control groups; a value greater than 1 indicates that the risk is greater in the diseased group; and a value less than 1 indicates that the risk is greater in the control group. In certain embodiments, IRC markers and/or IRC marker panels are selected to exhibit a hazard ratio of at least about 1.1 or more or about 0.91 or less, at least about 1.25 or more or about 0.8 or less, at least about 1.5 or more or about 0.67 or less, at least about 2 or more or about 0.5 or less, or at least about 2.5 or more or about 0.4 or less.

In some cases, multiple thresholds may be determined in so-called “tertile,” “quartile,” or “quintile” analyses. In these methods, the “diseased” and “control groups” (or “high risk” and “low risk”) groups are considered together as a single population, and are divided into 3, 4, or 5 (or more) “bins” having equal numbers of individuals. The boundary between two of these “bins” may be considered “thresholds.” A risk (of a particular diagnosis or prognosis for example) can be assigned based on which “bin” a test subject falls into.

In other embodiments, particular thresholds for the IRC marker(s) measured are not relied upon to determine if the marker level(s) obtained from a subject are correlated to a particular diagnosis or prognosis, For example, a temporal change in the marker(s) can be used to rule in or out one or more particular diagnoses and/or prognoses. Alternatively, IRC marker(s) are correlated to a condition, disease, prognosis, etc., by the presence or absence of the IRC marker(s) in a particular assay format. In the case of IRC marker panels, the present invention may utilize an evaluation of the entire profile of IRC markers to provide a single result value (e.g., a “panel response” value expressed either as a numeric score or as a percentage risk). In such embodiments, an increase, decrease, or other change (e.g., slope over time) in a certain subset of IRC markers may be sufficient to indicate a particular condition or future outcome in one patient, while an increase, decrease, or other change in a different subset of IRC markers may be sufficient to indicate the same or a different condition or outcome in another patient.

### 7. Methods of Detecting Aberrant IRC Marker Gene Expression

The present invention is predicated in part on the discovery that subjects with clinical evidence of sepsis, inSIRS and post-surgical inflammation have aberrant expression of certain genes (referred to herein as “IRC marker genes”) whose transcripts include, but are not limited to: SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99; 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513 or 515, as compared to one or more control subjects selected from normal (i.e., healthy) subjects, sepsis-negative subjects, inSIRS-negative subjects, post surgical inflammation-negative subjects, sepsis-negative, inSIRS-negative subjects, sepsis-negative, post surgical inflammation-negative subjects, inSIRS-negative, post surgical inflammation-negative subjects, sepsis-positive subjects, inSIRS-positive subjects and post-surgical inflammation-positive subject. In some embodiments, at least two subjects forming a control or reference population are used for the comparison. For example, the control or reference populations may be chosen from individuals who do not have post-surgical inflammation (“post-surgical inflammation-negative”), from individuals who do not have inSIRS (“inSIRS-negative”), from individuals who do not have inSIRS but who are suffering from an infectious process, from individuals who are suffering from post-surgical inflammation without the presence of inSIRS or sepsis (“post-surgical inflammation-positive”), from individuals who are suffering from inSIRS without the presence of sepsis (“inSIRS-positive”), from individuals who are suffering from the onset of sepsis, from individuals who are sepsis-positive and suffering from one of the stages in the progression of sepsis, or from individuals with a physiological trauma that increases the risk of developing sepsis. The control or reference populations may be post-surgical inflammation-positive and are subsequently diagnosed with inSIRS using conventional techniques. For example, a population of post-surgical inflammation-positive patients used to generate the reference profile may be diagnosed with inSIRS about 24, 48, 72, 96 or more hours after biological samples are taken from them for the purposes of generating a reference IRC marker profile. In some embodiments, the population of post-surgical inflammation-positive individuals is diagnosed with inSIRS using conventional techniques about 0-36 hours, about 36-60 hours, about 60-84 hours, or about 84-108 hours after the biological samples are taken. If the marker profile is indicative of inSIRS or one of its stages of progression, a clinician may begin treatment prior to the manifestation of clinical symptoms.

In other embodiments, the control or reference populations may be inSIRS-positive and are subsequently diagnosed with sepsis using convention techniques. For example, a population of inSIRS-positive patients used to generate the reference profile may be diagnosed with sepsis about 24, 48, 72, 96 or more hours after biological samples are taken from them for the purposes of generating a reference IRC marker profile. In some embodiments, the population of inSIRS-positive individuals is diagnosed with sepsis using conventional techniques about 0-36 hours, about 36-60 hours, about 60-84 hours, or about 84-108 hours after the biological samples are taken. If the marker profile is indicative of sepsis or one of its stages of progression, a clinician may begin treatment prior to the manifestation of clinical symptoms of sepsis. Treatment typically will involve examining the patient to determine the source of the infection. Once locating the source, the clinician typically will obtain cultures from the site of the infection, suitably before beginning relevant empirical antimicrobial therapy and perhaps additional adjunctive therapeutic measures, such as draining an abscess or removing an infected catheter.

In accordance with the present invention, comparing the level of at one IRC marker expression product in a subject to the level of a corresponding IRC marker expression product in a control subject selected for example from a normal subject, a sepsis-positive subject, an inSIRS-positive subjects and a post-surgical inflammation-positive subject indicates whether the subject under test is normal or has or is at risk of developing post-surgical inflammation, inSIRS or sepsis.

Accordingly, in certain embodiments, the invention features a method for diagnosing the presence or absence of a plurality of conditions selected from post-surgical inflammation, inSIRS or sepsis, or for distinguishing between those conditions in a subject by detecting differential expression of an IRC marker expression product between a test subject and a control subject. Accordingly, in order to make such diagnoses, it is desirable to qualitatively or quantitatively determine the levels of IRC marker transcripts or the level or functional activity of IRC marker polypeptides. In some embodiments, the presence or absence of post-surgical inflammation, inSIRS or sepsis, or differentiation between post-surgical inflammation, inSIRS and sepsis, is determined when an IRC marker expression product is expressed at a detectably lower level in a biological sample obtained from the test subject than the level at which a corresponding IRC expression product is expressed in a reference sample obtained from a control subject. In other embodiments, the presence or absence of post-surgical inflammation, inSIRS or sepsis, or differentiation between post-surgical inflammation, inSIRS and sepsis, is determined when na IRC marker expression product is expressed at a detectably higher level in a biological sample obtained from the test subject than the level at which a corresponding IRC expression product is expressed in a reference sample obtained from a control subject. Generally, such diagnoses are made when the level or functional activity of an IRC marker expression product in the biological sample varies from the level or functional activity of a corresponding IRC marker expression product in the reference sample by at least 10%, 20%, 30%, 40%, 50%, 60%, 70%, 80%, 90%, 92%, 94%, 96%, 97%, 98% or 99%, or even by at least about 99.5%, 99.9%, 99.95%, 99.99%, 99.995% or 99.999%, or even by at least about 100%, 200%, 300%, 400%, 500%, 600%, 700%, 800%, 900% or 1000%. The corresponding IRC marker expression product is generally selected from the same IRC marker expression product that is present in the biological sample, an IRC expression product expressed from a variant gene (e.g., an homologous or orthologous gene) including an allelic variant, or a splice variant or protein product thereof. In some embodiments, the method comprises measuring the level of 1, 2, 3, 4, 5, 6, 7, 8, 9, 10, 11, 12, 13, 14, 15, 16, 17, 18, 19, 20, 21, 22, 23 or 24 IRC marker expression products from an IRC multi-transcript-producing gene selected from ANKDD1A, GABRR2, OTX1, PANX2, RHBDF2, SLAMF7, AMBRA1, CES2, CLPB, HIPK2, C1ORF91, NDST1, SLC36A1, ADAM19, CUL7, TG, PDCD1LG2, GRINL1A, MGRN1, SNTB2, CDK5R1, GAA, KATNAL2, CEACAM4, ZNF335, ASPHD2, ACRC, BTNL8, MOV10, MED12L, KLHL6, PDLIM5, GALNT10, SCRN1, VOPP1, FKBP9, KIF27, PIWIL4, TEP1, GCH1, PRR11, CDH2, PPM1N, RRAS, DDOST, APH1A, TTL, TEX261, COQ2, FCHSD1, BAK1, SLC25A25, RELT, ACP2, TBC1D2B, FANCA or SLC39A11, either alone or in combination with as much as 1, 2, 3, 4, 5, 6, 7, 8, 9, 10, 11, 12, 13, 14, 15, 16, 17, 18, 19, 20, 21, 22, 23 or 24 individual IRC marker expression products from each of 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 19, 18, 17, 16, 15, 14, 13, 12, 11, 10, 9, 8, 7, 6, 5, 4, 3 or 2 IRC multi-transcript-producing genes or from 1 other IRC multi-transcript-producing gene.

In other embodiments, the methods comprise measuring the level of one or more IRC marker polypeptides from at least one IRC multi-transcript-producing gene as defined herein, either alone or in combination with as much as 1, 2, 3, 4, 5, 6, 7, 8, 9, 10, 11 or 12 individual IRC marker polypeptides expressed from 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 19, 18, 17, 16, 15, 14, 13, 12, 11, 10, 9, 8, 7, 6, 5, 4, 3, 2 or 1 other IRC multi-transcript-producing gene(s).

Generally, the biological sample contains blood, especially peripheral blood, or a fraction or extract thereof. Typically, the biological sample comprises blood cells such as mature, immature and developing leukocytes, including lymphocytes, polymorphonuclear leukocytes, neutrophils, monocytes, reticulocytes, basophils, coelomocytes, hemocytes, eosinophils, megakaryocytes, macrophages, dendritic cells natural killer cells, or fraction of such cells (e.g., a nucleic acid or protein fraction). In specific embodiments, the biological sample comprises leukocytes including peripheral blood mononuclear cells (PBMC).

7.1 Nucleic Acid-Based Diagnostics

Nucleic acid used in polynucleotide-based assays can be isolated from cells contained in the biological sample, according to standard methodologies (Sambrook, et al., 1989, supra; and Ausubel et al., 1994, supra). The nucleic acid is typically fractionated (e.g., poly A⁺ RNA) or whole cell RNA. Where RNA is used as the subject of detection, it may be desired to convert the RNA to a complementary DNA. In some embodiments, the nucleic acid is amplified by a template-dependent nucleic acid amplification technique. A number of template dependent processes are available to amplify the IRC marker sequences present in a given template sample. An exemplary nucleic acid amplification technique is the polymerase chain reaction (referred to as PCR) which is described in detail in U.S. Pat. Nos. 4,683,195, 4,683,202 and 4,800,159, Ausubel et al. (supra), and in Innis et al., (“PCR Protocols”, Academic Press, Inc., San Diego Calif., 1990). Briefly, in PCR, two primer sequences are prepared that are complementary to regions on opposite complementary strands of the marker sequence. An excess of deoxynucleoside triphosphates are added to a reaction mixture along with a DNA polymerase, e.g., Taq polymerase. If a cognate IRC marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated. A reverse transcriptase PCR amplification procedure may be performed in order to quantify the amount of mRNA amplified. Methods of reverse transcribing RNA into cDNA are well known and described in Sambrook et al., 1989, supra. Alternative methods for reverse transcription utilize thermostable, RNA-dependent DNA polymerases. These methods are described in WO 90/07641. Polymerase chain reaction methodologies are well known in the art.

In certain advantageous embodiments, the template-dependent amplification involves quantification of transcripts in real-time. For example, RNA or DNA may be quantified using the Real-Time PCR technique (Higuchi, 1992, et al., Biotechnology 10: 413-417). By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundance of the specific mRNA from which the target sequence was derived can be determined for the respective tissues or cells. This direct proportionality between the concentration of the PCR products and the relative mRNA abundance is only true in the linear range of the PCR reaction. The final concentration of the target DNA in the plateau portion of the curve is determined by the availability of reagents in the reaction mix and is independent of the original concentration of target DNA. In specific embodiments, multiplexed, tandem PCR (MT-PCR) is employed, which uses a two-step process for gene expression profiling from small quantities of RNA or DNA, as described for example in US Pat. Appl. Pub. No. 20070190540. In the first step, RNA is converted into cDNA and amplified using multiplexed gene specific primers. In the second step each individual gene is quantitated by real time PCR.

Another method for amplification is the ligase chain reaction (“LCR”), disclosed in EPO No. 320 308. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Qβ Replicase, described in PCT Application No. PCT/US87/00880, may also be used. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′α-thio-triphosphates in one strand of a restriction site may also be useful in the amplification of nucleic acids in the present invention, Walker et al., (1992, Proc. Natl. Acad. Sci. U.S.A 89: 392-396).

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification method described in GB Application No. 2 202 328, and in PCT Application No. PCT/US89/01025, may be used. In the former application, “modified” primers are used in a PCR-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labelled probe signals the presence of the target sequence.

Other nucleic acid amplification procedures include transcription-based amplification systems (TAS), including nucleic acid sequence based amplification (NASBA) and 3SR (Kwoh et al., 1989, Proc. Natl. Acad. Sci. U.S.A., 86: 1173; Gingeras et al., PCT Application WO 88/10315). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by an RNA polymerase such as T7 or SP6. In an isothermal cyclic reaction, the RNAs are reverse transcribed into single stranded DNA, which is then converted to double stranded DNA, and then transcribed once again with an RNA polymerase such as T7 or SP6. The resulting products, whether truncated or complete, indicate target specific sequences.

Vincent and Kong disclose a method termed helicase-dependent isothermal DNA amplification (HDA) (Vincent and Kong, EMBO Reports, 5(8):795-800, 2004). This method uses DNA helicase to separate DNA strands and hence does not require thermal cycling. The entire reaction can be carried out at one temperature and this method should have broad application to point-of-care DNA diagnostics.

Davey et al., EPO No. 329 822 disclose a nucleic acid amplification process involving cyclically synthesizing single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al. in PCT Application WO 89/06700 disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, M. A., In: “PCR Protocols: A Guide to Methods and Applications”, Academic Press, N.Y., 1990; Ohara et al., 1989, Proc. Natl. Acad. Sci. U.S.A., 86: 5673-567).

Methods based on ligation of two (or more) oligonucleotides in the presence of nucleic acid having the sequence of the resulting “di-oligonucleotide”, thereby amplifying the di-oligonucleotide, may also be used for amplifying target nucleic acid sequences. Wu et al., (1989, Genomics 4: 560).

Depending on the format, the IRC marker nucleic acid of interest is identified in the sample directly using a template-dependent amplification as described, for example, above, or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994, J Macromol. Sci. Pure, Appl. Chem., A31(1): 1355-1376).

In some embodiments, amplification products or “amplicons” are visualized in order to confirm amplification of the IRC marker sequences. One typical visualization method involves staining of a gel with ethidium bromide and visualization under UV light. Alternatively, if the amplification products are integrally labeled with radio- or fluorometrically-labelled nucleotides, the amplification products can then be exposed to x-ray film or visualized under the appropriate stimulating spectra, following separation. In some embodiments, visualization is achieved indirectly. Following separation of amplification products, a labeled nucleic acid probe is brought into contact with the amplified IRC marker sequence. The probe is suitably conjugated to a chromophore but may be radiolabeled. Alternatively, the probe is conjugated to a binding partner, such as an antigen-binding molecule, or biotin, and the other member of the binding pair carries a detectable moiety or reporter molecule. The techniques involved are well known to those of skill in the art and can be found in many standard texts on molecular protocols (e.g., see Sambrook et al., 1989, supra and Ausubel et al. 1994, supra). For example, chromophore or radiolabel probes or primers identify the target during or following amplification.

In certain embodiments, target nucleic acids are quantified using blotting techniques, which are well known to those of skill in the art. Southern blotting involves the use of DNA as a target, whereas Northern blotting involves the use of RNA as a target. Each provide different types of information, although cDNA blotting is analogous, in many aspects, to blotting or RNA species. Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter. Subsequently, the blotted target is incubated with a probe (usually labeled) under conditions that promote denaturation and rehybridization. Because the probe is designed to base pair with the target, the probe will bind a portion of the target sequence under renaturing conditions. Unbound probe is then removed, and detection is accomplished as described above.

Following detection/quantification, one may compare the results seen in a given subject with a control reaction or a statistically significant reference group or population of control subjects as defined herein. In this way, it is possible to correlate the amount of a IRC marker nucleic acid detected with the progression or severity of the disease.

Also contemplated are genotyping methods and allelic discrimination methods and technologies such as those described by Kristensen et al. (Biotechniques 30(2): 318-322), including the use of single nucleotide polymorphism analysis, high performance liquid chromatography, TaqMan®, liquid chromatography, and mass spectrometry.

Also contemplated are biochip-based technologies such as those described by Hacia et al. (1996, Nature Genetics 14: 441-447) and Shoemaker et al. (1996, Nature Genetics 14: 450-456). Briefly, these techniques involve quantitative methods for analysing large numbers of genes rapidly and accurately. By tagging genes with oligonucleotides or using fixed probe arrays, one can employ biochip technology to segregate target molecules as high density arrays and screen these molecules on the basis of hybridization. See also Pease et al. (1994, Proc. Natl. Acad. Sci. U.S.A. 91: 5022-5026); Fodor et al. (1991, Science 251: 767-773). Briefly, nucleic acid probes to IRC marker polynucleotides are made and attached to biochips to be used in screening and diagnostic methods, as outlined herein. The nucleic acid probes attached to the biochip are designed to be substantially complementary to specific expressed IRC marker nucleic acids, i.e., the target sequence (either the target sequence of the sample or to other probe sequences, for example in sandwich assays), such that hybridization of the target sequence and the probes of the present invention occurs. This complementarity need not be perfect; there may be any number of base pair mismatches which will interfere with hybridization between the target sequence and the nucleic acid probes of the present invention. However, if the number of mismatches is so great that no hybridization can occur under even the least stringent of hybridization conditions, the sequence is not a complementary target sequence. In certain embodiments, more than one probe per sequence is used, with either overlapping probes or probes to different sections of the target being used. That is, two, three, four or more probes, with three being desirable, are used to build in a redundancy for a particular target. The probes can be overlapping (i.e. have some sequence in common), or separate.

As will be appreciated by those of ordinary skill in the art, nucleic acids can be attached to or immobilized on a solid support in a wide variety of ways. By “immobilized” and grammatical equivalents herein is meant the association or binding between the nucleic acid probe and the solid support is sufficient to be stable under the conditions of binding, washing, analysis, and removal as outlined below. The binding can be covalent or non-covalent. By “non-covalent binding” and grammatical equivalents herein is meant one or more of either electrostatic, hydrophilic, and hydrophobic interactions. Included in non-covalent binding is the covalent attachment of a molecule, such as, streptavidin to the support and the non-covalent binding of the biotinylated probe to the streptavidin. By “covalent binding” and grammatical equivalents herein is meant that the two moieties, the solid support and the probe, are attached by at least one bond, including sigma bonds, pi bonds and coordination bonds. Covalent bonds can be formed directly between the probe and the solid support or can be formed by a cross linker or by inclusion of a specific reactive group on either the solid support or the probe or both molecules. Immobilization may also involve a combination of covalent and non-covalent interactions.

In general, the probes are attached to the biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid or semi-solid substrate or solid support. By “substrate” or “solid support” is meant any material that can be modified to contain discrete individual sites appropriate for the attachment or association of the nucleic acid probes and is amenable to at least one detection method. As will be appreciated by practitioners in the art, the number of possible substrates are very large, and include, but are not limited to, glass and modified or functionalised glass, plastics (including acrylics, polystyrene and copolymers of styrene and other materials, polypropylene, polyethylene, polybutylene, polyurethanes, Teflon™, etc.), polysaccharides, nylon or nitrocellulose, resins, silica or silica-based materials including silicon and modified silicon, carbon, metals, inorganic glasses, plastics, etc. In general, the substrates allow optical detection and do not appreciably fluorescese.

Generally the substrate is planar, although as will be appreciated by those of skill in the art, other configurations of substrates may be used as well. For example, the probes may be placed on the inside surface of a tube, for flow-through sample analysis to minimize sample volume. Similarly, the substrate may be flexible, such as a flexible foam, including closed cell foams made of particular plastics.

In certain embodiments, oligonucleotides probes are synthesized on the substrate, as is known in the art. For example, photoactivation techniques utilizing photopolymerisation compounds and techniques can be used. In an illustrative example, the nucleic acids are synthesized in situ, using well known photolithographic techniques, such as those described in WO 95/25116; WO 95/35505; U.S. Pat. Nos. 5,700,637 and 5,445,934; and references cited within; these methods of attachment form the basis of the Affymetrix GeneChip™ technology.

In an illustrative biochip analysis, oligonucleotide probes on the biochip are exposed to or contacted with a nucleic acid sample suspected of containing one or more IRC marker polynucleotides under conditions favoring specific hybridization. Sample extracts of DNA or RNA, either single or double-stranded, may be prepared from fluid suspensions of biological materials, or by grinding biological materials, or following a cell lysis step which includes, but is not limited to, lysis effected by treatment with SDS (or other detergents), osmotic shock, guanidinium isothiocyanate and lysozyme. Suitable DNA, which may be used in the method of the invention, includes cDNA. Such DNA may be prepared by any one of a number of commonly used protocols as for example described in Ausubel, et al., 1994, supra, and Sambrook, et al., et al., 1989, supra.

Suitable RNA, which may be used in the method of the invention, includes messenger RNA, complementary RNA transcribed from DNA (cRNA) or genomic or subgenomic RNA. Such RNA may be prepared using standard protocols as for example described in the relevant sections of Ausubel, et al. 1994, supra and Sambrook, et al. 1989, supra).

cDNA may be fragmented, for example, by sonication or by treatment with restriction endonucleases. Suitably, cDNA is fragmented such that resultant DNA fragments are of a length greater than the length of the immobilized oligonucleotide probe(s) but small enough to allow rapid access thereto under suitable hybridization conditions. Alternatively, fragments of cDNA may be selected and amplified using a suitable nucleotide amplification technique, as described for example above, involving appropriate random or specific primers.

Usually the target IRC marker polynucleotides are detectably labeled so that their hybridization to individual probes can be determined. The target polynucleotides are typically detectably labeled with a reporter molecule illustrative examples of which include chromogens, catalysts, enzymes, fluorochromes, chemiluminescent molecules, bioluminescent molecules, lanthanide ions (e.g., Eu³⁴), a radioisotope and a direct visual label. In the case of a direct visual label, use may be made of a colloidal metallic or non-metallic particle, a dye particle, an enzyme or a substrate, an organic polymer, a latex particle, a liposome, or other vesicle containing a signal producing substance and the like. Illustrative labels of this type include large colloids, for example, metal colloids such as those from gold, selenium, silver, tin and titanium oxide. In some embodiments in which an enzyme is used as a direct visual label, biotinylated bases are incorporated into a target polynucleotide. Hybridization is detected by incubation with streptavidin-reporter molecules.

Suitable fluorochromes include, but are not limited to, fluorescein isothiocyanate (FITC), tetramethylrhodamine isothiocyanate (TRITC), R-Phycoerythrin (RPE), and Texas Red. Other exemplary fluorochromes include those discussed by Dower et al. (International Publication WO 93/06121). Reference also may be made to the fluorochromes described in U.S. Pat. Nos. 5,573,909 (Singer et al), 5,326,692 (Brinkley et al). Alternatively, reference may be made to the fluorochromes described in U.S. Pat. Nos. 5,227,487, 5,274,113, 5,405,975, 5,433,896, 5,442,045, 5,451,663, 5,453,517, 5,459,276, 5,516,864, 5,648,270 and 5,723,218. Commercially available fluorescent labels include, for example, fluorescein phosphoramidites such as Fluoreprime™ (Pharmacia), Fluoredite™ (Millipore) and FAM (Applied Biosystems International)

Radioactive reporter molecules include, for example, ³²P, which can be detected by an X-ray or phosphoimager techniques.

The hybrid-forming step can be performed under suitable conditions for hybridizing oligonucleotide probes to test nucleic acid including DNA or RNA. In this regard, reference may be made, for example, to NUCLEIC ACID HYBRIDIZATION, A PRACTICAL APPROACH (Homes and Higgins, eds.) (IRL press, Washington D.C., 1985). In general, whether hybridization takes place is influenced by the length of the oligonucleotide probe and the polynucleotide sequence under test, the pH, the temperature, the concentration of mono- and divalent cations, the proportion of G and C nucleotides in the hybrid-forming region, the viscosity of the medium and the possible presence of denaturants. Such variables also influence the time required for hybridization. The preferred conditions will therefore depend upon the particular application. Such empirical conditions, however, can be routinely determined without undue experimentation.

In certain advantageous embodiments, high discrimination hybridization conditions are used. For example, reference may be made to Wallace et al. (1979, Nucl. Acids Res. 6: 3543) who describe conditions that differentiate the hybridization of 11 to 17 base long oligonucleotide probes that match perfectly and are completely homologous to a target sequence as compared to similar oligonucleotide probes that contain a single internal base pair mismatch. Reference also may be made to Wood et al. (1985, Proc. Natl. Acid. Sci. USA 82: 1585) who describe conditions for hybridization of 11 to 20 base long oligonucleotides using 3M tetramethyl ammonium chloride wherein the melting point of the hybrid depends only on the length of the oligonucleotide probe, regardless of its GC content. In addition, Drmanac et al. (supra) describe hybridization conditions that allow stringent hybridization of 6-10 nucleotide long oligomers, and similar conditions may be obtained most readily by using nucleotide analogues such as ‘locked nucleic acids (Christensen et al., 2001, Biochem J 354: 481-4).

Generally, a hybridization reaction can be performed in the presence of a hybridization buffer that optionally includes a hybridization-optimizing agent, such as an isostabilising agent, a denaturing agent and/or a renaturation accelerant. Examples of isostabilising agents include, but are not restricted to, betaines and lower tetraalkyl ammonium salts. Denaturing agents are compositions that lower the melting temperature of double stranded nucleic acid molecules by interfering with hydrogen bonding between bases in a double stranded nucleic acid or the hydration of nucleic acid molecules. Denaturing agents include, but are not restricted to, formamide, formaldehyde, dimethylsulfoxide, tetraethyl acetate, urea, guanidium isothiocyanate, glycerol and chaotropic salts. Hybridization accelerants include heterogeneous nuclear ribonucleoprotein (hnRP) A1 and cationic detergents such as cetyltrimethylammonium bromide (CTAB) and dodecyl trimethylammonium bromide (DTAB), polylysine, spermine, spermidine, single stranded binding protein (SSB), phage T4 gene 32 protein and a mixture of ammonium acetate and ethanol. Hybridization buffers may include target polynucleotides at a concentration between about 0.005 nM and about 50 nM, preferably between about 0.5 nM and 5 nM, more preferably between about 1 nM and 2 nM.

A hybridization mixture containing the target IRC marker polynucleotides is placed in contact with the array of probes and incubated at a temperature and for a time appropriate to permit hybridization between the target sequences in the target polynucleotides and any complementary probes. Contact can take place in any suitable container, for example, a dish or a cell designed to hold the solid support on which the probes are bound. Generally, incubation will be at temperatures normally used for hybridization of nucleic acids, for example, between about 20° C. and about 75° C., example, about 25° C., about 30° C., about 35° C., about 40° C., about 45° C., about 50° C., about 55° C., about 60° C., or about 65° C. For probes longer than 14 nucleotides, 20° C. to 50° C. is desirable. For shorter probes, lower temperatures are preferred. A sample of target polynucleotides is incubated with the probes for a time sufficient to allow the desired level of hybridization between the target sequences in the target polynucleotides and any complementary probes. For example, the hybridization may be carried out at about 45° C.+/−10° C. in formamide for 1-2 days.

After the hybrid-forming step, the probes are washed to remove any unbound nucleic acid with a hybridization buffer, which can typically comprise a hybridization optimizing agent in the same range of concentrations as for the hybridization step. This washing step leaves only bound target polynucleotides. The probes are then examined to identify which probes have hybridized to a target polynucleotide.

The hybridization reactions are then detected to determine which of the probes has hybridized to a corresponding target sequence. Depending on the nature of the reporter molecule associated with a target polynucleotide, a signal may be instrumentally detected by irradiating a fluorescent label with light and detecting fluorescence in a fluorimeter; by providing for an enzyme system to produce a dye which could be detected using a spectrophotometer; or detection of a dye particle or a colored colloidal metallic or non metallic particle using a reflectometer; in the case of using a radioactive label or chemiluminescent molecule employing a radiation counter or autoradiography. Accordingly, a detection means may be adapted to detect or scan light associated with the label which light may include fluorescent, luminescent, focussed beam or laser light. In such a case, a charge couple device (CCD) or a photocell can be used to scan for emission of light from a probe:target polynucleotide hybrid from each location in the micro-array and record the data directly in a digital computer. In some cases, electronic detection of the signal may not be necessary. For example, with enzymatically generated color spots associated with nucleic acid array format, visual examination of the array will allow interpretation of the pattern on the array. In the case of a nucleic acid array, the detection means is suitably interfaced with pattern recognition software to convert the pattern of signals from the array into a plain language genetic profile. In certain embodiments, oligonucleotide probes specific for different IRC marker polynucleotides are in the form of a nucleic acid array and detection of a signal generated from a reporter molecule on the array is performed using a ‘chip reader’. A detection system that can be used by a ‘chip reader’ is described for example by Pirrung et al (U.S. Pat. No. 5,143,854). The chip reader will typically also incorporate some signal processing to determine whether the signal at a particular array position or feature is a true positive or maybe a spurious signal. Exemplary chip readers are described for example by Fodor et al (U.S. Pat. No., 5,925,525). Alternatively, when the array is made using a mixture of individually addressable kinds of labeled microbeads, the reaction may be detected using flow cytometry.

7.2 Protein-Based Diagnostics

Consistent with the present invention, a difference in concentration of a IRC marker protein between a test subject or sample and a control subject or reference sample is indicative of the presence or absence of sepsis or inSIRS or distinguishes between sepsis and inSIRS. IRC marker protein levels in biological samples can be assayed using any suitable method known in the art. For example, when a IRC marker protein is an enzyme, the protein can be quantified based upon its catalytic activity or based upon the number of molecules of the protein contained in a sample. Antibody-based techniques may be employed, such as, for example, immunohistological and immunohistochemical methods for measuring the level of a protein of interest in a tissue sample. For example, specific recognition is provided by a primary antibody (polyclonal or monoclonal) and a secondary detection system is used to detect presence (or binding) of the primary antibody. Detectable labels can be conjugated to the secondary antibody, such as a fluorescent label, a radiolabel, or an enzyme (e.g., alkaline phosphatase, horseradish peroxidase) which produces a quantifiable, e.g., colored, product. In another suitable method, the primary antibody itself can be detectably labeled. As a result, immunohistological labeling of a tissue section is provided. In some embodiments, a protein extract is produced from a biological sample (e.g., tissue, cells) for analysis. Such an extract (e.g., a detergent extract) can be subjected to western-blot or dot/slot assay of the level of the protein of interest, using routine immunoblotting methods (Jalkanen et al., 1985, J. Cell. Biol. 101: 976-985; Jalkanen et al., 1987, J. Cell. Biol. 105: 3087-3096).

Other useful antibody-based methods include immunoassays, such as the enzyme-linked immunosorbent assay (ELISA) and the radioimmunoassay (MA). For example, a protein-specific monoclonal antibody, can be used both as an immunoadsorbent and as an enzyme-labeled probe to detect and quantify a IRC marker protein of interest. The amount of such protein present in a sample can be calculated by reference to the amount present in a standard preparation using a linear regression computer algorithm (see Lacobilli et al., 1988, Breast Cancer Research and Treatment 11: 19-30). In other embodiments, two different monoclonal antibodies to the protein of interest can be employed, one as the immunoadsorbent and the other as an enzyme-labeled probe.

Additionally, recent developments in the field of protein capture arrays permit the simultaneous detection and/or quantification of a large number of proteins. For example, low-density protein arrays on filter membranes, such as the universal protein array system (Ge, 2000 Nucleic Acids Res. 28(2):e3) allow imaging of arrayed antigens using standard ELISA techniques and a scanning charge-coupled device (CCD) detector. Immuno-sensor arrays have also been developed that enable the simultaneous detection of clinical analytes. It is now possible using protein arrays, to profile protein expression in bodily fluids, such as in sera of healthy or diseased subjects, as well as in subjects pre- and post-drug treatment.

Protein capture arrays typically comprise a plurality of protein-capture agents each of which defines a spatially distinct feature of the array. The protein-capture agent can be any molecule or complex of molecules which has the ability to bind a protein and immobilize it to the site of the protein-capture agent on the array. The protein-capture agent may be a protein whose natural function in a cell is to specifically bind another protein, such as an antibody or a receptor. Alternatively, the protein-capture agent may instead be a partially or wholly synthetic or recombinant protein which specifically binds a protein. Alternatively, the protein-capture agent may be a protein which has been selected in vitro from a mutagenized, randomized, or completely random and synthetic library by its binding affinity to a specific protein or peptide target. The selection method used may optionally have been a display method such as ribosome display or phage display, as known in the art. Alternatively, the protein-capture agent obtained via in vitro selection may be a DNA or RNA aptamer which specifically binds a protein target (see, e.g., Potyrailo et al., 1998 Anal. Chem. 70:3419-3425; Cohen et al., 1998, Proc. Natl. Acad. Sci. USA 95:14272-14277; Fukuda, et al., 1997 Nucleic Acids Symp. Ser. 37:237-238; available from SomaLogic). For example, aptamers are selected from libraries of oligonucleotides by the Selex™ process and their interaction with protein can be enhanced by covalent attachment, through incorporation of brominated deoxyuridine and UV-activated crosslinking (photoaptamers). Aptamers have the advantages of ease of production by automated oligonucleotide synthesis and the stability and robustness of DNA; universal fluorescent protein stains can be used to detect binding. Alternatively, the in vitro selected protein-capture agent may be a polypeptide (e.g., an antigen) (see, e.g., Roberts and Szostak, 1997 Proc. Natl. Acad. Sci. USA 94:12297-12302).

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins which have the appropriate primary amino acid sequence (e.g., available from ProteinPrint™ and Aspira Biosystems).

Exemplary protein capture arrays include arrays comprising spatially addressed antigen-binding molecules, commonly referred to as antibody arrays, which can facilitate extensive parallel analysis of numerous proteins defining a proteome or subproteome. Antibody arrays have been shown to have the required properties of specificity and acceptable background, and some are available commercially (e.g., BD Biosciences, Clontech, BioRad and Sigma). Various methods for the preparation of antibody arrays have been reported (see, e.g., Lopez et al., 2003 J. Chromatogr. B 787:19-27; Cahill, 2000 Trends in Biotechnology 7:47-51; U.S. Pat. App. Pub. 2002/0055186; U.S. Pat. App. Pub. 2003/0003599; PCT publication WO 03/062444; PCT publication WO 03/077851; PCT publication WO 02/59601; PCT publication WO 02/39120; PCT publication WO 01/79849; PCT publication WO 99/39210). The antigen-binding molecules of such arrays may recognise at least a subset of proteins expressed by a cell or population of cells, illustrative examples of which include growth factor receptors, hormone receptors, neurotransmitter receptors, catecholamine receptors, amino acid derivative receptors, cytokine receptors, extracellular matrix receptors, antibodies, lectins, cytokines, serpins, proteases, kinases, phosphatases, ras-like GTPases, hydrolases, steroid hormone receptors, transcription factors, heat-shock transcription factors, DNA-binding proteins, zinc-finger proteins, leucine-zipper proteins, homeodomain proteins, intracellular signal transduction modulators and effectors, apoptosis-related factors, DNA synthesis factors, DNA repair factors, DNA recombination factors, cell-surface antigens, hepatitis C virus (HCV) proteases and HIV proteases.

Antigen-binding molecules for antibody arrays are made either by conventional immunization (e.g., polyclonal sera and hybridomas), or as recombinant fragments, usually expressed in E. coli, after selection from phage display or ribosome display libraries (e.g., available from Cambridge Antibody Technology, Bioinvent, Affitech and Biosite). Alternatively, ‘combibodies’ comprising non-covalent associations of VH and VL domains, can be produced in a matrix format created from combinations of diabody-producing bacterial clones (e.g., available from Domantis). Exemplary antigen-binding molecules for use as protein-capture agents include monoclonal antibodies, polyclonal antibodies, Fv, Fab, Fab′ and F(ab′)₂ immunoglobulin fragments, synthetic stabilized Fv fragments, e.g., single chain Fv fragments (scFv), disulfide stabilized Fv fragments (dsFv), single variable region domains (dAbs) minibodies, combibodies and multivalent antibodies such as diabodies and multi-scFv, single domains from camelids or engineered human equivalents.

Individual spatially distinct protein-capture agents are typically attached to a support surface, which is generally planar or contoured. Common physical supports include glass slides, silicon, microwells, nitrocellulose or PVDF membranes, and magnetic and other microbeads.

While microdrops of protein delivered onto planar surfaces are widely used, related alternative architectures include CD centrifugation devices based on developments in microfluidics (e.g., available from Gyros) and specialized chip designs, such as engineered microchannels in a plate (e.g., The Living Chip™, available from Biotrove) and tiny 3D posts on a silicon surface (e.g., available from Zyomyx).

Particles in suspension can also be used as the basis of arrays, providing they are coded for identification; systems include color coding for microbeads (e.g., available from Luminex, Bio-Rad and Nanomics Biosystems) and semiconductor nanocrystals (e.g., QDots™, available from Quantum Dots), and barcoding for beads (UltraPlex™, available from Smartbeads) and multimetal microrods (Nanobarcodes™ particles, available from Surromed). Beads can also be assembled into planar arrays on semiconductor chips (e.g., available from LEAPS technology and BioArray Solutions). Where particles are used, individual protein-capture agents are typically attached to an individual particle to provide the spatial definition or separation of the array. The particles may then be assayed separately, but in parallel, in a compartmentalized way, for example in the wells of a microtiter plate or in separate test tubes.

In operation, a protein sample, which is optionally fragmented to form peptide fragments (see, e.g., U.S. Pat. App. Pub. 2002/0055186), is delivered to a protein-capture array under conditions suitable for protein or peptide binding, and the array is washed to remove unbound or non-specifically bound components of the sample from the array. Next, the presence or amount of protein or peptide bound to each feature of the array is detected using a suitable detection system. The amount of protein bound to a feature of the array may be determined relative to the amount of a second protein bound to a second feature of the array. In certain embodiments, the amount of the second protein in the sample is already known or known to be invariant.

For analyzing differential expression of proteins between two cells or cell populations, a protein sample of a first cell or population of cells is delivered to the array under conditions suitable for protein binding. In an analogous manner, a protein sample of a second cell or population of cells to a second array, is delivered to a second array which is identical to the first array. Both arrays are then washed to remove unbound or non-specifically bound components of the sample from the arrays. In a final step, the amounts of protein remaining bound to the features of the first array are compared to the amounts of protein remaining bound to the corresponding features of the second array. To determine the differential protein expression pattern of the two cells or populations of cells, the amount of protein bound to individual features of the first array is subtracted from the amount of protein bound to the corresponding features of the second array.

In an illustrative example, fluorescence labeling can be used for detecting protein bound to the array. The same instrumentation as used for reading DNA microarrays is applicable to protein-capture arrays. For differential display, capture arrays (e.g. antibody arrays) can be probed with fluorescently labeled proteins from two different cell states, in which cell lysates are labeled with different fluorophores (e.g., Cy-3 and Cy-5) and mixed, such that the color acts as a readout for changes in target abundance. Fluorescent readout sensitivity can be amplified 10-100 fold by tyramide signal amplification (TSA) (e.g., available from PerkinElmer Lifesciences). Planar waveguide technology (e.g., available from Zeptosens) enables ultrasensitive fluorescence detection, with the additional advantage of no washing procedures. High sensitivity can also be achieved with suspension beads and particles, using phycoerythrin as label (e.g., available from Luminex) or the properties of semiconductor nanocrystals (e.g., available from Quantum Dot). Fluorescence resonance energy transfer has been adapted to detect binding of unlabelled ligands, which may be useful on arrays (e.g., available from Affibody). Several alternative readouts have been developed, including adaptations of surface plasmon resonance (e.g., available from HTS Biosystems and Intrinsic Bioprobes), rolling circle DNA amplification (e.g., available from Molecular Staging), mass spectrometry (e.g., available from Sense Proteomic, Ciphergen, Intrinsic and Bioprobes), resonance light scattering (e.g., available from Genicon Sciences) and atomic force microscopy (e.g., available from BioForce Laboratories). A microfluidics system for automated sample incubation with arrays on glass slides and washing has been co-developed by NextGen and Perkin Elmer Life Sciences.

In certain embodiments, the techniques used for detection of IRC marker expression products will include internal or external standards to permit quantitative or semi-quantitative determination of those products, to thereby enable a valid comparison of the level or functional activity of these expression products in a biological sample with the corresponding expression products in a reference sample or samples. Such standards can be determined by the skilled practitioner using standard protocols. In specific examples, absolute values for the level or functional activity of individual expression products are determined.

In specific embodiments, the diagnostic methods are implemented using a system as disclosed, for example, in International Publication No. WO 02/090579 and in copending PCT Application No. PCT/AU03/01517 filed Nov. 14, 2003, comprising at least one end station coupled to a base station. The base station is typically coupled to one or more databases comprising predetermined data from a number of individuals representing the level or functional activity of IRC marker expression products, together with indications of the actual status of the individuals (e.g., presence, absence of sepsis or inSIRS or post-surgical inflammation) when the predetermined data was collected. In operation, the base station is adapted to receive from the endstation, typically via a communications network, subject data representing a measured or normalized level or functional activity of at least one expression product in a biological sample obtained from a test subject and to compare the subject data to the predetermined data stored in the database(s). Comparing the subject and predetermined data allows the base station to determine the status of the subject in accordance with the results of the comparison. Thus, the base station attempts to identify individuals having similar parameter values to the test subject and once the status has been determined on the basis of that identification, the base station provides an indication of the diagnosis to the end station.

7.3 Kits

All the essential materials and reagents required for detecting and quantifying IRC marker expression products may be assembled together in a kit. The kits may also optionally include appropriate reagents for detection of labels, positive and negative controls, washing solutions, blotting membranes, microtiter plates dilution buffers and the like. For example, a nucleic acid-based detection kit may include (i) an IRC marker polynucleotide (which may be used as a positive control), (ii) a primer or probe that specifically hybridizes to an IRC marker polynucleotide. Also included may be enzymes suitable for amplifying nucleic acids including various polymerases (Reverse Transcriptase, Taq, Sequenase™ DNA ligase etc. depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe. Alternatively, a protein-based detection kit may include (i) an IRC marker polypeptide (which may be used as a positive control), (ii) an antigen-binding molecule that is immuno-interactive with an IRC marker polypeptide. The kit can also feature various devices and reagents for performing one of the assays described herein; and/or printed instructions for using the kit to quantify the expression of an sepsis marker gene.

### 8. Methods of Treatment or Prophylaxis

The present invention also extends to the management of post-surgical inflammation, inSIRS and sepsis, or prevention of further progression of post-surgical inflammation, inSIRS and sepsis, or assessment of the efficacy of therapies in subjects following positive diagnosis for the presence of post-surgical inflammation, inSIRS or sepsis in a subject. Post-surgical inflammation is typically managed using intravenous fluids, anti-inflammatories, antibiotics or immunotherapy. However, the management of sepsis or inSIRS conditions is generally highly intensive and can include identification and amelioration of the underlying cause and aggressive use of therapeutic compounds such as, vasoactive compounds, antibiotics, steroids, antibodies to endotoxin, anti tumour necrosis factor agents, recombinant protein C. In addition, palliative therapies as described for example in Cohen and Glauser (1991, Lancet 338: 736-739) aimed at restoring and protecting organ function can be used such as intravenous fluids and oxygen and tight glycemic control. Therapies for sepsis are reviewed in Healy (2002, Ann. Pharmacother. 36(4): 648-54) and Brindley (2005, CJEM. 7(4): 227) and Jenkins (2006, J Hosp Med. 1(5): 285-295).

Typically, the therapeutic agents will be administered in pharmaceutical (or veterinary) compositions together with a pharmaceutically acceptable carrier and in an effective amount to achieve their intended purpose. The dose of active compounds administered to a subject should be sufficient to achieve a beneficial response in the subject over time such as a reduction in, or relief from, the symptoms of post-surgical inflammation, sepsis or inSIRS. The quantity of the pharmaceutically active compounds(s) to be administered may depend on the subject to be treated inclusive of the age, sex, weight and general health condition thereof. In this regard, precise amounts of the active compound(s) for administration will depend on the judgment of the practitioner. In determining the effective amount of the active compound(s) to be administered in the treatment or prevention of post-surgical inflammation, sepsis or inSIRS, the medical practitioner or veterinarian may evaluate severity of any symptom associated with the presence of post-surgical inflammation, sepsis or inSIRS including, inflammation, blood pressure anomaly, tachycardia, tachypnea fever, chills, vomiting, diarrhoea, skin rash, headaches, confusion, muscle aches, seizures. In any event, those of skill in the art may readily determine suitable dosages of the therapeutic agents and suitable treatment regimens without undue experimentation.

The therapeutic agents may be administered in concert with adjunctive (palliative) therapies to increase oxygen supply to major organs, increase blood flow to major organs and/or to reduce the inflammatory response. Illustrative examples of such adjunctive therapies include non steroidal-anti inflammatory drugs (NSAIDs), intravenous saline and oxygen.

In order that the invention may be readily understood and put into practical effect, particular preferred embodiments will now be described by way of the following non-limiting examples.

### EXAMPLES

### Example 1

### Identification of Diagnostic Genes that Distinguish Between Post-Surgical Inflammation, Sepsis and inSIRS

### Experimental Disease Trial Designs

Clinical trials were performed to determine whether transcripts of genes could distinguish between patients with sepsis, inSIRS and post-surgical inflammation.

Blood samples were collected at various time points to provide time course data and gene expression was analysed using an Affymetrix exon array (Affymetrix HuEx-1.0) Analysis of these data (see “Identification of Diagnostic Marker Genes” below) revealed 235 specific genes that show evidence of splice variation that also differ in expression between sepsis-positive patients, inSIRS-positive patients and post-surgical patients. Of these 235 only a limited number (57) were identified that can be used as classifiers to distinguish between these patient groups. The 57 genes produce 258 transcripts that are differentially expressed between post-surgical inflammation and inSIRS, post-surgical inflammation and sepsis and sepsis and inSIRS. It is possible to design a nucleic acid assay that measures the RNA level in the sample corresponding to at least one and desirably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 39, 40, 41, 42, 43, 44, 45, 46, 47, 48, 49, 50, 51, 52, 53, 64, 55, 56, 57 IRC marker transcripts, representative transcript sequences of which are set forth in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513 or 515. Alternatively, or in addition, it is possible to design an assay that measures the protein level in the sample corresponding to at least one and desirably at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 39, 40, 41, 42, 43, 44, 45, 46, 47, 48, 49, 50, 51, 52, 53, 64, 55, 56, 57 IRC marker polypeptides, representative amino acid sequences of which are set forth in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514 or 516.

### Materials and Methods

### Study Design

In Phase I of a sepsis clinical research program, the trial was conducted at a single tertiary referral centre. Intensive care sepsis patients, as well as healthy controls were prospectively enrolled and attended a single visit where 5 mL of blood was collected for gene expression analysis using an Affymetrix exon array.

A definitive diagnosis of sepsis was unlikely to be known at the time patients were enrolled in the Phase II study, and thus confirmation of sepsis diagnosis and the assignment of patients to the sepsis cohorts was made retrospectively. Data collected from participants not diagnosed with inSIRS or sepsis were only assessed for frequency of adverse events.

Clinical data collection and blood specimens were not collected until after surgery during their post-operative admission to the ICU. Patients who had clinical signs and/or symptoms of sepsis or inSIRS were consented and enrolled into the study as soon as possible after they have been identified, in most cases within 24 hours of admission. Final assessment of whether the participant had inSIRS or sepsis or post-surgical inflammation was made retrospectively as clinical information became available.

### Study Population

In the Phase I clinical trial, 12 patients presenting with clinical signs and symptoms of sepsis (SIRS criteria as well as suspected infection) from the ICU were enrolled. A further 10 male and 10 female control participants from within were also enrolled. Study participants were all over 18 years and either they or their surrogate decision maker signed and dated the clinical trial information sheet and consent form. All of the control participants were considered to be in good health based on an abbreviated physical examination by the Principal Investigator, and known medical history at the time of clinical trial enrolment.

Phase II of this clinical research program comprised of two cohorts of patients and included a cohort of 36 inSIRS patients with clinical signs and symptoms consistent with inSIRS, and a cohort of 17 patients with the clinical signs and symptoms consistent with sepsis.

Patients or their surrogate decision maker were offered the opportunity to participate in this study if the patient presented with signs and symptoms of either inSIRS or sepsis at the time of admission to ICU. All inSIRS and sepsis participants displayed clinical signs of fever, hypotension, leukocytosis or leucopoenia and decreased peripheral vascular resistance. These criteria are based on the American College of Physicians and the Society of Critical Care Medicine standard definition of sepsis. That is, inSIRS and sepsis participants needed a variable combination of clinical conditions including two or more of the following within the last 24 hours: temperature >38° C. or <36° C.; heart rate >90 beats/min; respiratory rate >20 breathes/min or a PaCO₂ of <4.3 kPa (<32 mm Hg); and evidence of a white blood cell count <4,000 cells/mm³ (<4×10⁹ cells/L) or >12,000 cells/mm³ (>12×10⁹ cells/L) or >10% immature neutrophils (band forms). Participants were excluded if they had any chronic systemic immune-inflammatory disorders including SLE, Crohn's disease, IDDM; were transplant recipients or were currently receiving chemotherapy treatment for cancer. Most patients had other underlying co-morbidities. Patients who were admitted for planned major open surgery were approached and consented prior to the surgical procedure, and their study blood samples drawn after surgery during their post-operative admission to the ICU. All study participants were 18 years of age or older and had a body mass index of less than 40.

### Collection of Data

Demography, vital signs measurements (blood pressure, heart rate, respiratory rate, oxygen saturation, temperature), hematology (full blood count), clinical chemistry. (urea, electrolytes, liver function enzymes, blood glucose) as well as microbial status was recorded. Blood was drawn into maximally 6 PAXgene tubes for gene expression analysis using RT-PCR.

### Blood Collection

Blood is collected for the purpose of extraction of high quality RNA or protein. Suitable blood collection tubes for the collection, preservation, transport and isolation of RNA include PAXgene™ tubes (PreAnalytix Inc., Valencia, Calif., USA). Alternatively, blood can be collected into tubes containing solutions designed for the preservation of nucleic acids (available from Roche, Ambion, Invitrogen and ABI). For the determination of protein levels, 50 mL of blood is prevented from clotting by collection into a tube containing 4 mL of 4% sodium citrate. White blood cells and plasma are isolated and stored frozen for later analysis and detection of specific proteins. PAXgene tubes can be kept at room temperature prior to RNA extraction. Clinical signs are recorded in a standard format.

### Total RNA Extraction

A kit available from Qiagen Inc (Valencia, Calif., USA) has the reagents and instructions for the isolation of total RNA from 2.5 mL blood collected in the PAXgene Blood RNA Tube. Isolation begins with a centrifugation step to pellet nucleic acids in the PAXgene blood RNA tube. The pellet is washed and resuspended and incubated in optimized buffers together with Proteinase K to bring about protein digestion. An additional centrifugation is carried out to remove residual cell debris and the supernatant is transferred to a fresh microcentrifuge tube. Ethanol is added to adjust binding conditions, and the lysate is applied to the PAXgene RNA spin column. During brief centrifugation, RNA is selectively bound to the silica-gel membrane as contaminants pass through. Remaining contaminants are removed in three efficient wash steps and RNA is then eluted in Buffer BR5.

Determination of RNA quantity and quality is necessary prior to proceeding and can be achieved using an Agilent Bioanalyzer and Absorbance 260/280 ratio using a spectrophotometer.

### Choice of Method

Measurement of specific RNA levels in a tissue sample can be achieved using a variety of technologies. Two common and readily available technologies that are well known in the art are:
GeneChip® analysis using Affymetrix technology.
Real-Time Polymerase Chain Reaction (TaqMan™ from Applied Biosystems for example).

GeneChips® quantitate RNA by detection of labeled cRNA hybridized to short oligonucleotides built on a silicon substrate. Details on the technology and methodology can be found at www.affymetrix.com.

Real-Time Polymerase Chain Reaction (RT-PCR) quantitates RNA using two PCR primers, a labeled probe and a thermostable DNA polymerase. As PCR product is generated a dye is released into solution and detected. Internal controls such as 18S RNA probes are often used to determine starting levels of total RNA in the sample. Each gene and the internal control are run separately. Details on the technology and methods can be found at www.appliedbiosytems.com or www.qiagen.com or www.biorad.com. Applied Biosystems offer a service whereby the customer provides DNA sequence information and payment and is supplied in return all of the reagents required to perform RT-PCR analysis on individual genes.

GeneChip® analysis has the advantage of being able to analyze thousands of genes at a time. However it is expensive and takes over 3 days to perform a single assay. RT-PCR generally only analyses one gene at a time, but is inexpensive and can be completed within a single day.

RT-PCR is the method, of choice for gene expression analysis if the number of specific genes to be analyzed is less than 20. GeneChip® or other gene expression analysis technologies (such as Illumina Bead Arrays) are the method of choice when many genes need to be analyzed simultaneously.

The methodology for GeneChip® data generation and analysis and Real Time PCR is presented below in brief.

### GeneChip® Data Generation

### cDNA & cRNA Generation

The following method for cDNA and cRNA generation from total RNA has been adapted from the protocol provided and recommended by Affymetrix (www.affymetrix.com).

The steps are:
A total of 3 μg of total RNA is used as a template to generate double stranded cDNA.
cRNA is generated and labeled using biotinylated Uracil (dUTP).
biotin-labeled cRNA is cleaned and the quantity determined using a spectrophotometer and MOPS gel analysis.
labeled cRNA is fragmented to ˜300 bp in size.
RNA quantity is determined on an Agilent “Lab-on-a-Chip” system (Agilent Technologies).

### Hybridization, Washing & Staining

The steps are:
A hybridization cocktail is prepared containing 0.05 μg/μL of labeled and fragmented cRNA, spike-in positive hybridization controls, and the Affymetrix oligonucleotides B2, bioB, bioC, bioD and cre.
The final volume (80 μL) of the hybridization cocktail is added to the GeneChip® cartridge.
The cartridge is placed in a hybridization oven at constant rotation for 16 hours.
The fluid is removed from the GeneChip® and stored.
The GeneChip® is placed in the fluidics station.
The experimental conditions for each GeneChip® are recorded as an .EXP file.
All washing and staining procedures are carried out by the Affymetrix fluidics station with an attendant providing the appropriate solutions.
The GeneChip® is washed, stained with steptavidin-phycoerythin dye and then washed again using low salt solutions.
After the wash protocols are completed, the dye on the probe array is ‘excited’ by laser and the image captured by a CCD camera using an Affymetrix Scanner (manufactured by Agilent).

### Scanning & Data File Generation

The scanner and MAS 5 software generates an image file from a single GeneChip® called a .DAT file.

The .DAT file is then pre-processed prior to any statistical analysis.

Data pre-processing steps (prior to any statistical analysis) include:
.DAT File Quality Control (QC).
.CEL File Generation.
Scaling and Normalization.

### .DAT File Quality Control

The .DAT file is an image. The image is inspected manually for artifacts (e.g. high/low intensity spots, scratches, high regional or overall background). (The B2 oligonucleotide hybridization performance is easily identified by an alternating pattern of intensities creating a border and array name.) The MAS 5 software used the B2 oligonucleotide border to align a grid over the image so that each square of oligonucleotides was centered and identified.

The other spiked hybridization controls (bioB, bioC, bioD and cre) are used to evaluate sample hybridization efficiency by reading “present” gene detection calls with increasing signal values, reflecting their relative concentrations. (If the .DAT file is of suitable quality it is converted to an intensity data file (.CEL file) by Affymetrix MAS 5 software).

### .CEL File Generation

The .CEL files generated by the MAS 5 software from .DAT files contain calculated raw intensities for the probe sets. Gene expression data is obtained by subtracting a calculated background from each cell value. To eliminate negative intensity values, a noise correction fraction based from a local noise value from the standard deviation of the lowest 2% of the background is applied.

All .CEL files generated from the GeneChips® are subjected to specific quality metrics parameters.

Some metrics are routinely recommended by Affymetrix and can be determined from Affymetrix internal controls provided as part of the GeneChip®. Other metrics are based on experience and the processing of many GeneChips®.

Analysis of GeneChip® Data

Two illustrative approaches to normalising data may be used:
Affymetrix MAS 5 Algorithm.
Robust Multi-chip Analysis (RMA) algorithm of Irizarry (Irizarray et al., 2002, Biostatistics (in print)).

Those of skill in the art will recognize that many other approaches might be adopted, without materially affecting the invention.

### Preprocessing

The arrays were preprocessed using the Affymetrix Power Tools (APT) apt-probeset-summarize program. The analysis used the array description files current at the time, (\HuEx-1 0-st-v2.r2.pgf″ and \HuEx-1 0-st-v2.r2.clf″), the antigenomic probes for background (\HuEx-1 0-st-v2.r2.antigenomic.bgp″) and the standard QC probes (\HuEx-1 0-st-v2.r2.qcc″). Additionally, in all the analyses, the Robust Multichip Average (RMA) approach was used.

Using various Affymetrix mapping files, it is possible to compute expression measures at either the Exon or Gene level, and for subsets of Exons or Genes entitled, Core, Extend or Full. To date in subsequent analysis the focus has been on the Core set of Exons and Genes as these are the most well understood and annotated subsets. There is an exon analysis package available for the R statistical software package (www.r-project.org) called exonmap. It is provided by the X:Map genome browser project (http://xmap.picr.man.ac.uk). Exonmap provides an alternative chip description \exon.pmcdf″ that can be used to produce exon level RMA normalised measures of expression for the Core set of exons. On comparison with the output of the APT utilities, the differences were found to be minor. Since the APT utilities also provide gene level measures, these version were used throughout.

### Quality Checking

The APT utility provides various quality control summaries including the use of boxplots of the mean expression levels for the positive and negative controls.

### Model for the Data

The data were analysed to identify differential features (exons or genes) using the linear model approach embodied in the limma package of R. limma proceeds by estimating the coefficients for each feature and computing a moderated t-statistic for each contrast of interest. In addition, an overall F-statistic is computed for the 3 contrasts together. The equivalent p-values can then be adjusted for multiple tests in various ways. In this case, Holm's method of adjustment and Benjamini & Hochberg's false discovery rate (FDR).

### Affymetrix MAS 5 Algorithm

.CEL files are used by Affymetrix MAS 5 software to normalize or scale the data. Scaled data from one chip are compared to similarly scaled data from other chips.

Affymetrix MAS 5 normalization is achieved by applying the default “Global Scaling” option of the MAS 5 algorithm to the .CEL files. This procedure subtracts a robust estimate of the center of the distribution of probe values, and divides by a robust estimate of the probe variability. This produces a set of chips with common location and scale at the probe level.

Gene expression indices are generated by a robust averaging procedure on all the probe pairs for a given gene. The results are constrained to be non-negative.

Given that scaling takes place at the level of the probe, rather than at the level of the gene, it is possible that even after normalization there may be chip-to-chip differences in overall gene expression level. Following standard MAS5 normalization, values for each gene were de-trended with respect to median chip intensity. That is, values for each gene were regressed on the median chip intensity, and residuals were calculated. These residuals were taken as the de-trended estimates of expression for each gene

Median chip intensity was calculated using the Affymetrix MAS5 algorithm, but with a scale factor fixed at one.

### RMA Algorithm

This algorithm quantifies the expression of a set of chips, rather than of a single chip. It estimates background intensities using a robust statistical model applied to perfect match probe data. It does not make use of mis-match probe data. Following implicit background correction, chips are processed using Quantile Quantile normalization (Rizarray et al., 2002, Biostatistics (in print)).

### DNA Extraction

A kit available from Qiagen Inc (Valencia, Calif., USA) has the reagents and instructions for the isolation of total DNA from 8.5 mL blood collected in the PAXgene Blood DNA Tube. Isolation begins with the addition of additional lysis solution followed by a centrifugation step. The pellet is washed and resuspended and incubated in optimized buffers together with Proteinase K to bring about protein digestion. DNA is precipitated using alcohol and an additional centrifugation is carried out to pellet the nucleic acid. Remaining contaminants are removed in a wash step and the DNA is then resuspended in Buffer BG4.

Determination of DNA quantity and quality is necessary prior to proceeding and can be achieved using a spectrophotometer or agarose gel electrophoresis.

### Genotyping Analysis

Many methods are available to genotype DNA. A review of allelic discrimination methods can be found in Kristensen et al. (Biotechniques 30(2): 318-322 (2001). An illustrative method for genotyping using allele-specific PCR is described here.

### Primer Design

Upstream and downstream PCR primers specific for particular alleles can be designed using freely available computer programs, such as Primer3 (http://frodo.wi.mit.edu/primer3/primer3_code.html). Alternatively the DNA sequences of the various alleles can be aligned using a program such as ClustalW (http://www.ebi.ac.uk/clustalw/) and specific primers designed to areas where DNA sequence differences exist but retaining enough specificity to ensure amplification of the correct amplicon. Preferably a PCR amplicon is designed to have a restriction enzyme site in one allele but not the other. Primers are generally 18-25 base pairs in length with similar melting temperatures.

### PCR Amplification

The composition of PCR reactions has been described elsewhere (Clinical Applications of PCR, Dennis Lo (Editor), Blackwell Publishing, 1998). Briefly, a reaction contains primers, DNA, buffers and a thermostable polymerase enzyme. The reaction is cycled (up to 50 times) through temperature steps of denaturation, hybridization and DNA extension on a thermocycler such as the MJ Research Thermocycler model PTC-96V.

### DNA Analysis

PCR products can be analyzed using a variety of methods including size differentiation using mass spectrometry, capillary gel electrophoresis and agarose gel electrophoresis. If the PCR amplicons have been designed to contain differential restriction enzyme sites, the DNA in the PCR reaction is purified using DNA-binding columns or precipitation and re-suspended in water, and then restricted using the appropriate restriction enzyme. The restricted DNA can then be run on an agarose gel where DNA is separated by size using electric current. Various alleles of a gene will have different sizes depending on whether they contain restriction sites. Thus, homozygotes and heterozygotes can be determined.

### Real-Time PCR Data Generation

Background information for conducting Real-time PCR may be obtained, for example, at http://dorakmt.tripod.com/genetics/realtime.html and in a review by Bustin SA (2000, J Mol Endocrinol 25:169-193).

### TaqMan™ Primer and Probe Design Guidelines

1. The Primer Express™ (ABI) software designs primers with a melting temperature (Tm) of 58-60° C., and probes with a Tm value of 10° C. higher. The Tm of both primers should be equal.

2. Primers should be 15-30 bases in length.

3. The G+C content should ideally be 30-80%. If a higher G+C content is unavoidable, the use of high annealing and melting temperatures, cosolvents such as glycerol, DMSO, or 7-deaza-dGTP may be necessary:

4. The run of an identical nucleotide should be avoided. This is especially true for G, where runs of four or more Gs is not allowed.

5. The total number of Gs and Cs in the last five nucleotides at the 3′ end of the primer should not exceed two (the newer version of the software has an option to do this automatically). This helps to introduce relative instability to the 3′ end of primers to reduce non-specific priming. The primer conditions are the same for SYBR Green assays.

6. Maximum amplicon size should not exceed 400 by (ideally 50-150 bases). Smaller amplicons give more consistent results because PCR is more efficient and more tolerant of reaction conditions (the short length requirement has nothing to do with the efficiency of 5′ nuclease activity).

7. The probes should not have runs of identical nucleotides (especially four or more consecutive Gs), G+C content should be 30-80%, there should be more Cs than Gs, and not a G at the 5′ end. The higher number of Cs produces a higher αRn. The choice of probe should be made first.

8. To avoid false-positive results due to amplification of contaminating genomic DNA in the cDNA preparation, it is preferable to have primers spanning exon-exon junctions. This way, genomic DNA will not be amplified (the PDAR kit for human GAPDH amplification has such primers),

9. If a TaqMan™ probe is designed for allelic discrimination, the mismatching nucleotide (the polymorphic site) should be in the middle of the probe rather than at the ends,

10. Use primers that contain dA nucleotides near the 3′ ends so that any primer-dimer generated is efficiently degraded by AmpErase™ UNG (mentioned in p. 9 of the manual for EZ RT-PCR kit; P/N402877). If primers cannot be selected with dA nucleotides near the ends, the use of primers with 3′ terminal dU-nucleotides should be considered.

(See also the general principles of PCR Primer Design by InVitroGen.)

### General Method

1. Reverse transcription of total RNA to cDNA should be done with random hexamers (not with oligo-dT). If oligo-dT has to be used long mRNA transcripts or amplicons greater than two kilobases upstream should be avoided, and 18S RNA cannot be used as normalizer,

2. Multiplex PCR will only work properly if the control primers are limiting (ABI control reagents do not have their primers limited),

3. The range of target cDNA used is 10 ng to 1 μg. If DNA is used (mainly for allelic discrimination studies), the optimum amount is 100 ng to 1 μg,

4. It is ideal to treat each RNA preparation with RNAse free DNAse to avoid genomic DNA contamination. Even the best RNA extraction methods yield some genomic DNA. Of course, it is ideal to have primers not amplifying genomic DNA at all but sometimes this may not be possible,

5. For optimal results, the reagents (before the preparation of the PCR mix) and the PCR mixture itself (before loading) should be vortexed and mixed well. Otherwise there may be shifting Rn value during the early (0-5) cycles of PCR. It is also important to add probe to the buffer component and allow it to equilibrate at room temperature prior to reagent mix formulation.

### TaqMan™ Primers and Probes

The TaqMan™ probes ordered from ABI at midi-scale arrive already resuspended at 100 □M. If a 1/20 dilution is made, this gives a 5 μM solution. This stock solution should be aliquoted, frozen and kept in the dark. Using 1 μL of this in a 50 μL reaction gives the recommended 100 nM final concentration.

The primers arrive lyophilized with the amount given on the tube in pmols (such as 150.000 pmol which is equal to 150 nmol). If X nmol of primer is resuspended in X μL of H2O, the resulting solution is 1 mM. It is best to freeze this stock solution in aliquots. When the 1 mM stock solution is diluted 1/100, the resulting working solution will be 10 μM. To get the recommended 50-900 nM final primer concentration in 50 μL reaction volume, 0.25-4.50 □L should be used per reaction (2.5 μL for 500 nM final concentration).

The PDAR primers and probes are supplied as a mix in one tube. They have to be used 2.5 μL in a 50 μL reaction volume.

### Setting Up One-Step TaqMan™ Reaction

One-step real-time PCR uses RNA (as opposed to cDNA) as a template. This is the preferred method if the RNA solution has a low concentration but only if singleplex reactions are run. The disadvantage is that RNA carryover prevention enzyme AmpErase cannot be used in one-step reaction format. In this method, both reverse transcriptase and real-time PCR take place in the same tube. The downstream PCR primer also acts as the primer for reverse transcriptase (random hexamers or oligo-dT cannot be used for reverse transcription in one-step RT-PCR). One-step reaction requires higher dNTP concentration (greater than or equal to 300 mM vs 200 mM) as it combines two reactions needing dNTPs in one. A typical reaction mix for one-step PCR by Gold RT-PCR kit is as follows:

| | |
|---|---|
| H₂O + RNA: | 20.5 μL [24 μL if PDAR is used] |
| 10X TaqMan buffer: | 5.0 μL |
| MgCl₂ (25 mM): | 11.0 μL |
| dATP (10 mM): | 1.5 μL [for final concentration of 300 μM] |
| dCTP (10 mM): | 1.5 μL [for final concentration of 300 μM] |
| dGTP (10 mM): | 1.5 μL [for final concentration of 300 μM] |
| dUTP (20 mM): | 1.5 μL [for final concentration of 600 μM] |
| Primer F (10 μM) *: | 2.5 μL [for final concentration of 500 nM] |
| Primer R (10 μM) *: | 2.5 μL [for final concentration of 500 nM] |
| TaqMan Probe *: | 1.0 μL [for final concentration of 100 nM] |
| AmpliTaq Gold: | 0.25 μL [can be increased for higher efficiency] |
| Reverse Transcriptase: | 0.25 μL |
| RNAse inhibitor: | 1.00 μL |

* If a PDAR is used, 2.5 μL of primer + probe mix used.

Ideally 10 pg-100 ng RNA should be used in this reaction. Note that decreasing the amount of template from 100 ng to 50 ng will increase the C_{T} value by 1. To decrease a C_{T} value by 3, the initial amount of template should be increased 8-fold. ABI claims that 2 picograms of RNA can be detected by this system and the maximum amount of RNA that can be used is 1 microgram. For routine analysis, 10 pg-100 ng RNA and 100 pg-1 μg genomic DNA can be used.

### Cycling Parameters for One-Step PCR

Reverse transcription (by MuLV) 48° C. for 30 min.

AmpliTaq activation 95° C. for 10 min.

PCR: denaturation 95° C. for 15 sec and annealing/extension 60° C. for 1 min (repeated 40 times) (On ABI 7700, minimum holding time is 15 seconds.)

The recently introduced EZ One-Step™ RT-PCR kit allows the use of UNG as the incubation time for reverse transcription is 60° C. thanks to the use of a thermostable reverse transcriptase. This temperature also a better option to avoid primer dimers and non-specific bindings at 48° C.

### Operating the ABI 7700

Make sure the following before starting a run:

1. Cycle parameters are correct for the run.

2. Choice of spectral compensation is correct (off for singleplex, on for multiplex reactions).

3. Choice of “Number of PCR Stages” is correct in the Analysis Options box (Analysis/Options). This may have to be manually assigned after a run if the data is absent in the amplification plot but visible in the plate view, and the X-axis of the amplification is displaying a range of 0-1 cycles.

4. No Template Control is labeled as such (for accurate □Rn calculations).

5. The choice of dye component should be made correctly before data analysis.

6. You must save the run before it starts by giving it a name (not leaving as untitled). Also at the end of the run, first save the data before starting to analyze.

7. The ABI software requires extreme caution. Do not attempt to stop a run after clicking on the Run button. You will have problems and if you need to switch off and on the machine, you have to wait for at least an hour to restart the run.

When analyzing the data, remember that the default setting for baseline is 3-15. If any CT value is <15, the baseline should be changed accordingly (the baseline stop value should be 1-2 smaller than the smallest CT value). For a useful discussion of this matter, see the ABI Tutorial on Setting Baselines and Thresholds. (Interestingly, this issue is best discussed in the manual for TaqMan™ Human Endogenous Control Plate.)

If the results do not make sense, check the raw spectra for a possible CDC camera saturation during the run. Saturation of CDC camera may be prevented by using optical caps rather than optical adhesive cover. It is also more likely to happen when SYBR Green I is used, when multiplexing and when a high concentration of probe is used.

### Interpretation of Results

At the end of each reaction, the recorded fluorescence intensity is used for the following calculations:

Rn+ is the Rn value of a reaction containing all components, Rn− is the Rn value of an unreacted sample (baseline value or the value detected in NTC). ΔRn is the difference between Rn+ and Rn−. It is an indicator of the magnitude of the signal generated by the PCR.

There are three illustrative methods to quantitate the amount of template:

1. Absolute standard method: In this method, a known amount of standard such as in vitro translated RNA (cRNA) is used.

2. Relative standard: Known amounts of the target nucleic acid are included in the assay design in each run,

3. Comparative CT method: This method uses no known amount of standard but compares the relative amount of the target sequence to any of the reference values chosen and the result is given as relative to the reference value (such as the expression level of resting lymphocytes or a standard cell line).

### The Comparative CT Method (ΔΔCT) for Relative Quantitation of Gene Expression

This method enables relative quantitation of template and increases sample throughput by eliminating the need for standard curves when looking at expression levels relative to an active reference control (normalizer). For this method to be successful, the dynamic range of both the target and reference should be similar. A sensitive method to control this is to look at how ΔC_{T} (the difference between the two C_{T} values of two PCRs for the same initial template amount) varies with template dilution. If the efficiencies of the two amplicons are approximately equal, the plot of log input amount versus ΔC_{T} will have a nearly horizontal line (a slope of <0.10). This means that both PCRs perform equally efficiently across the range of initial template amounts. If the plot shows unequal efficiency, the standard curve method should be used for quantitation of gene expression. The dynamic range should be determined for both (1) minimum and maximum concentrations of the targets for which the results are accurate and (2) minimum and maximum ratios of two gene quantities for which the results are accurate. In conventional competitive RT-PCR, the dynamic range is limited to a target-to-competitor ratio of about 10:1 to 1:10 (the best accuracy is obtained for 1:1 ratio). The real-time PCR is able to achieve a much wider dynamic range.

Running the target and endogenous control amplifications in separate tubes and using the standard curve method requires the least amount of optimization and validation. The advantage of using the comparative C_{T} method is that the need for a standard curve is eliminated (more wells are available for samples). It also eliminates the adverse effect of any dilution errors made in creating the standard curve samples.

As long as the target and normalizer have similar dynamic ranges, the comparative CT method (ΔΔC_{T} method) is the most practical method. It is expected that the normalizer will have a higher expression level than the target (thus, a smaller C_{T} value). The calculations for the quantitation start with getting the difference (ΔC_{T}) between the C_{T} values of the target and the normalizer:

ΔC_{T}=C_{T}(target)−C_{T}(normalizer)

This value is calculated for each sample to be quantitated (unless, the target is expressed at a higher level than the normalizer, this should be a positive value. It is no harm if it is negative). One of these samples should be chosen as the reference (baseline) for each comparison to be made. The comparative ΔΔC_{T} calculation involves finding the difference between each sample's ΔC_{T} and the baseline's ΔC_{T}. If the baseline value is representing the minimum level of expression, the ΔΔC_{T} values are expected to be negative (because the ΔC_{T} for the baseline sample will be the largest as it will have the greatest CT value). If the expression is increased in some samples and decreased in others, the ΔΔC_{T} values will be a mixture of negative and positive ones. The last step in quantitation is to transform these values to absolute values. The formula for this is:

comparative expression level=2−ΔΔC_{T}

For expressions increased compared to the baseline level this will be something like 23=8 times increase, and for decreased expression it will be something like 2-3=⅛ of the reference level. Microsoft Excel can be used to do these calculations by simply entering the CT values (there is an online ABI tutorial at http://www.appliedbiosystems.com/support/tutorials/7700 amp/ on the use of spread sheet programs to produce amplification plots; the TaqMan™ Human Endogenous Control Plate protocol also contains detailed instructions on using MS Excel for real-time PCR data analysis).

The other (absolute) quantification methods are outlined in the ABI User Bulletins (http://docs.appliedbiosystems.com/search.taf?_UserReference=A8658327189850A13A° C598E). The Bulletins #2 and #5 are most useful for the general understanding of real-time PCR and quantification.

Recommendations on Procedures:

1. Use positive-displacement pipettes to avoid inaccuracies in pipetting,

2. The sensitivity of real-time PCR allows detection of the target in 2 pg of total RNA. The number of copies of total RNA used in the reaction should ideally be enough to give a signal by 25-30 cycles (preferably less than 100 ng). The amount used should be decreased or increased to achieve this.

3. The optimal concentrations of the reagents are as follows:

i. Magnesium chloride concentration should be between 4 and 7 mM. It is optimized as 5.5 mM for the primers/probes designed using the Primer Express software.

ii. Concentrations of dNTPs should be balanced with the exception of dUTP (if used). Substitution of dUTP for dTTP for control of PCR product carryover requires twice dUTP that of other dNTPs. While the optimal range for dNTPs is 500 μM to 1 mM (for one-step RT-PCR), for a typical TaqMan reaction (PCR only), 200 μM of each dNTP (400 μM of dUTP) is used.

iii. Typically 0.25 μL (1.25 U) AmpliTaq DNA Polymerase (5.0 U/μL) is added into each 50 μL reaction. This is the minimum requirement. If necessary, optimization can be done by increasing this amount by 0.25 U increments.

iv. The optimal probe concentration is 50-200 nM, and the primer concentration is 100-900 nM. Ideally, each primer pair should be optimized at three different temperatures (58, 60 and 620 C for TaqMan primers) and at each combination of three concentrations (50, 300, 900 nM). This means setting up three different sets (for three temperatures) with nine reactions in each (50/50 mM, 50/300 mM, 50/900, 300/50, 300/300, 300/900, 900/50, 900/300, 900/900 mM) using a fixed amount of target template. If necessary, a second round of optimization may improve the results. Optimal performance is achieved by selecting the primer concentrations that provide the lowest CT and highest ΔRn. Similarly, the probe concentration should be optimized for 25-225 nM.

4. If AmpliTaq Gold DNA Polymerase is being used, there has to be a 9-12 min pre-PCR heat step at 92-950 C to activate it. If AmpliTaq Gold DNA Polymerase is used, there is no need to set up the reaction on ice. A typical TaqMan reaction consists of 2 min at 500 C for UNG (see below) incubation; 10 min at 95° C. for Polymerase activation, and 40 cycles of 15 sec at 95° C. (denaturation) and 1 min at 60° C. (annealing and extension). A typical reverse transcription cycle (for cDNA synthesis), which should precede the TaqMan reaction if the starting material is total RNA, consists of 10 min at 250 C (primer incubation), 30 min at 48° C. (reverse transcription with conventional reverse transcriptase) and 5 min at 95° C. (reverse transcriptase inactivation).

5. AmpErase uracil-N-glycosylase (UNG) is added in the reaction to prevent the reamplification of carry-over PCR products by removing any uracil incorporated into amplicons. This is why dUTP is used rather than dTTP in PCR reaction. UNG does not function above 55° C. and does not cut single-stranded DNA with terminal dU nucleotides. UNG-containing master mix should not be used with one-step RT-PCR unless rTth DNA polymerase is being used for reverse transcription and PCR (TaqMan EZ RT-PCR kit).

6. It is necessary to include at least three No Amplification Controls (NAC) as well as three No Template Controls (NTC) in each reaction plate (to achieve a 99.7% confidence level in the definition of +/−thresholds for the target amplification, six replicates of NTCs must be run). NAC former contains sample and no enzyme. It is necessary to rule out the presence of fluorescence contaminants in the sample or in the heat block of the thermal cycler (these would cause false positives). If the absolute fluorescence of the NAC is greater than that of the NTC after PCR, fluorescent contaminants may be present in the sample or in the heating block of the thermal cycler.

7. The dynamic range of a primer/probe system and its normalizer should be examined if the ΔΔCT method is going to be used for relative quantitation. This is done by running (in triplicate) reactions of five RNA concentrations (for example, 0, 80 pg/μL, 400 pg/μL, 2 ng/μL and 50 ng/μL). The resulting plot of log of the initial amount vs. CT values (standard curve) should be a (near) straight line for both the target and normalizer real-time RT-PCRs for the same range of total RNA concentrations.

8. The passive reference is a dye (ROX) included in the reaction (present in the TaqMan universal PCR master mix). It does not participate in the 5′ nuclease reaction. It provides an internal reference for background fluorescence emission. This is used to normalize the reporter-dye signal. This normalization is for non-PCR-related fluorescence fluctuations occurring well-to-well (concentration or volume differences) or over time and different from the normalization for the amount of cDNA or efficiency of the PCR. Normalization is achieved by dividing the emission intensity of reporter dye by the emission intensity of the passive reference. This gives the ratio defined as Rn.

9. If multiplexing is done, the more abundant of the targets will use up all the ingredients of the reaction before the other target gets a chance to amplify. To avoid this, the primer concentrations for the more abundant target should be limited.

10. TaqMan Universal PCR master mix should be stored at 2 to 8° C. (not at −20° C.).

11. The GAPDH probe supplied with the TaqMan Gold RT-PCR kit is labeled with a JOE reporter dye, the same probe provided within the Pre-Developed TaqMan™ Assay Reagents (PDAR) kit is labeled with VIC. Primers for these human GAPDH assays are designed not to amplify genomic DNA.

12. The carryover prevention enzyme, AmpErase UNG, cannot be used with one-step RT-PCR which requires incubation at 48° C. but may be used with the EZ RT-PCR kit.

13. One-step RT-PCR can only be used for singleplex reactions, and the only choice for reverse transcription is the downstream primer (not random hexamers or oligo-dT).

14. It is ideal to run duplicates to control pipetting errors but this inevitably increases the cost.

15. If multiplexing, the spectral compensation option (in Advanced Options) should be checked before the run.

16. Normalization for the fluorescent fluctuation by using a passive reference (ROX) in the reaction and for the amount of cDNA/PCR efficiency by using an endogenous control (such as GAPDH, active reference) are different processes.

17. ABI 7700 can be used not only for quantitative RT-PCR but also end-point PCR. The latter includes presence/absence assays or allelic discrimination assays (such as SNP typing).

18. Shifting Rn values during the early cycles (cycle 0-5) of PCR means initial disequilibrium of the reaction components and does not affect the final results as long as the lower value of baseline range is reset.

19. If an abnormal amplification plot has been noted (C_{T} value<15 cycles with amplification signal detected in early cycles), the upper value of the baseline range should be lowered and the samples should be diluted to increase the C_{T} value (a high C_{T} value may also be due to contamination).

20. A small ΔRn value (or greater than expected C_{T} value) indicates either poor PCR efficiency or low copy number of the target.

21. A standard deviation >0.16 for C_{T} value indicates inaccurate pipetting.

22. SYBR Green entry in the Pure Dye Setup should be abbreviated as “SYBR” in capitals. Any other abbreviation or lower case letters will cause problems.

23. The SDS software for ABI 7700 have conflicts with the Macintosh Operating System version 8.1. The data should not be analyzed on such computers.

24. The ABI 7700 should not be deactivated for extended periods of time. If it has ever been shutdown, it should be allowed to warm up for at least one hour before a run. Leaving the instrument on all times is recommended and is beneficial for the laser. If the machine has been switched on just before a run, an error box stating a firmware version conflict may appear. If this happens, choose the “Auto Download” option.

25. The ABI 7700 is only one of the real-time PCR systems available, others include systems from BioRad, Cepheid, Corbett Research, Roche and Stratagene.

### Example 2

### Determining Splice Variants

For a given gene, an anova approach to detecting splice variants was used. The approach taken was similar to the Affymetrix MIDAS approach. In the exon level data, there is an intensity for each probe set, for each subject. A simple model for the intensity would be an overall gene mean, plus a probe set effect plus a subject effect plus error. Where i indexes the probesets and j the subjects.

Yij=α+βi+γj+εij

This model applies only when there is no alternate splicing. If probe set i maps to exon e(i) and subject j is in class c(j) then alternate splicing would be represented by the presence of a term δe(i) c(j) in the model. In X:Map annotation, probe sets may match to multiple exons. This is associated with alternate exon layouts in the gene, so a test for a term δic(j), that is a probe set by class interaction, was performed. For simplicity, the subject effect was ignored (this variation becomes part of the noise).

### Example 3

### Gene Transcripts Distinguishing Sepsis from Post-Surgical Inflammation

Any of the gene transcripts in Table 7 are able to distinguish sepsis from post-surgical inflammation (the sign on values in the column logFC indicates comparative up or down regulation. By example, transcripts for ankdd1a can be expected to be relatively up-regulated in sepsis compared to post-surgical and transcripts for OTX1 can be expected to be relatively down-regulated in sepsis compared to post-surgical).

### Example 4

### Gene Transcripts Distinguishing Sepsis from inSIRS

Any of the gene transcripts in Table 8 are able to distinguish sepsis from inSIRS (the sign on values in the column logFC indicates comparative up or down regulation).

### Example 7

### Genes Distinguishing inSIRS from Post-Surgical

Gene transcripts in Table 9 may able to distinguish inSIRS and post-surgical inflammation (the sign on values in the column logFC indicates comparative up or down regulation).

### Example 8

### Area Under Curve (AUC) for Classifiers Separating Groups Using Exons from Splice Variants Using Several Statistical Techniques

Table 10 summarizes the area under the ROC curves (AAUC) as percentages. The closer to 100% these are the better the classifier.

It can be seen by looking at the percentage AUC for the various statistical techniques that Post-surgical inflammation versus Sepsis, Sepsis versus inSIRS, Post-surgical inflammation and inSIRS versus Sepsis and Post-surgical versus inflammation Sepsis and inSIRS provide good classifiers.

### Example 9

### Monitoring of Post-Surgical Patients

All surgery results in an acute phase response and inflammation and the severity of the response is proportional to the level of insult. Many cardiac surgery and abdominal surgery patients develop a bacterial translocation and endotoxemia which can lead to organ failure and death unless appropriately managed. In fact, it has been demonstrated that patients with pre-existing high plasma levels of anti-endotoxin antibody have a better survival rate compared to those patients with low anti-endotoxin plasma antibodies demonstrating that endotoxin and the immune response to endotoxin play a key role in survival in these patients. This post-surgical immune response often presents clinically as fever. Nurses and intensivists working with post-surgical patients with fever must therefore decide whether the cause of the fever relates to bacterial infection. The IRC biomarkers of the present invention, which are able to distinguish between post-surgical inflammation, SIRS and sepsis, would therefore be useful in determining an appropriate course of action in such patients which could include the use of antibiotics, anti-pyretics, immune modulators and/or anti-inflammatories. Monitoring such patients with these biomarkers would also allow for informed decisions on when to withdraw such treatments.

### Example 10

### Monitoring Trauma and Burns Patients

Severe trauma (especially head trauma) and burns patients have high levels of tissue damage and the resultant acute phase response and inflammation often causes swelling, fever and damage to vital organs such as the brain and skin. Such patients are often treated with steroids (or other anti-inflammatories) to reduce the level of inflammation which then makes then susceptible to bacterial infection. Brain damaged patients also often develop fevers. A therapeutic balancing act between the use of anti-inflammatories, immune modulating agents and antibiotics is therefore created in these patients. The IRC biomarkers of the present invention, which are able to distinguish between sterile inflammation and inflammation caused by bacterial infection, are therefore useful monitoring tools that are able to assist medical practitioners in determining appropriate therapies for the best outcome in such patients.

### Example 11

### Monitoring Patients in Intensive Care

Patients in intensive care are usually administered a number of therapeutic compounds—many of which have opposing actions on the immune system. Further, intensive care patients often have, or develop, inSIRS which can lead to multi-organ failure and death. Further still, intensive care patients often develop sepsis through hospital acquired infection. However, the ultimate aim of intensive care is to ensure the patient survives and is discharged to a general ward in the minimum time. The above factors confound this aim. Monitoring intensive care patients on a regular basis with the IRC biomarkers of the present invention will allow medical practitioners to: determine the level of inflammation, determine if the patient has a hospital acquired infection, and determine response to therapy. Information provided by these biomarkers will therefore allow medical practitioners to tailor and modify therapies to ensure patients survive and spend less time in intensive care. Less time in intensive care leads to considerable savings in medical expenses. In addition, informed use of antibiotics leads to less usage and further savings in medical expenses. Appropriate and informed use of antibiotics also leads to less antibiotic resistance.

### Example 12

### Patients with Fever—Distinguishing Between Inflammation, inSIRS and Sepsis

Many patients present to hospitals, or are in hospital, with fever of unknown origin. Fever can be caused by sterile inflammation or by microbial infection. The IRC biomarkers of the present invention, which are able to distinguish between inflammation, SIRS and sepsis, will be useful in screening, stratification, diagnosing and determining appropriate therapies in such patients.

### Example 13

### Determining the Severity of Immune Response to Insult

The IRC biomarkers disclosed herein are able to determine an inflammatory response continuum from the less severe inflammatory response of post-surgery through to the severe inflammatory response to bacterial infection (sepsis). Determining where a patient lies on this continuum is important with respect to deciding what therapies (if any) should be administered.

### Example 14

### Provision of a Prognosis

The IRC biomarkers of the present invention permit qualitative or quantitative grading of inflammatory response and provide a means to separate sepsis, inSIRS and post-surgical inflammation from each other. This, in turn, allows for the determination of a prognosis in patients determined to have any one of sepsis, inSIRS or post-surgical inflammation. It has been demonstrated that in-patients with inSIRS have a 6.9 times higher 28-day mortality compared to those without SIRS (Comstedt et al., 2007, Scand. J Trauma Resusc. Emerg. Med. 27: 17-67. 2009; Esteban et al., 2007, Crit. Care Med. 35(5): 1284-1289). Further, with respect to risk of dying, there is a graded severity from inSIRS to sepsis, severe sepsis and septic shock, with an associated 28-day mortality of approximately 10%, 20%, 20-40% and 40-60% respectively (Brun-Buisson, C., 2000, Intensive Care Medicine 26, Suppl 1: S64-74). Such information allows for informed decisions on choice of therapy and how aggressively to treat.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

Lengthy table referenced here
US20140037649A1-20140206-T00001
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20140037649A1). An electronic copy of the table will also be available from the USPTO upon request and payment of the fee set forth in 37 CFR 1.19(b)(3).

What is claimed is:

1. A method for assessing whether a subject has, or is at risk of developing, one of a plurality of conditions selected from sepsis, infection-negative SIRS (“inSIRS”) and post-surgical inflammation, the method comprising: comparing the level of at least one inflammatory response continuum (IRC) marker expression product of a multi-transcript-producing gene in the subject to the level of a corresponding IRC marker expression product in at least one control subject selected from: a post-surgical inflammation-positive subject, an inSIRS positive subject, a sepsis-positive subject and a normal subject, wherein a difference between the level of the at least one IRC marker expression product and the level of the corresponding IRC marker expression product indicates whether the subject has, or is at risk of developing, one of the conditions, wherein the at least one IRC marker expression product is predetermined as being differentially expressed between at least two of the conditions and wherein at least one other expression product from the multi-transcript producing gene is predetermined as being not so differentially expressed.

2. A method according to claim 1, wherein the multi-transcript-producing gene is selected from the group consisting of: ankyrin repeat and death domain containing 1A (ANKDD1A) gene, rho 2 (GABRR2) gene, orthodenticle homeobox 1 (OTX1) gene, pannexin 2 (PANX2) gene, rhomboid 5 homolog 2 (Drosophila) (RHBDF2) gene, SLAM family member 7 (SLAMF7) gene, autophagy/beclin-1 regulator 1 (AMBRA1) gene, carboxylesterase 2 (intestine, liver) (CES2) gene, caseinolytic peptidase B homolog (E. coli) (CLPB) gene, homeodomain interacting protein kinase 2 (HIPK2) gene and chromosome 1 open reading frame 91 (C1ORF91) gene, N-deacetylase/N-sulfotransferase (heparan glucosaminyl) 1 (NDST1) gene, solute carrier family 36 (proton/amino acid symporter) (member 1 (SLC36A1) gene, ADAM metallopeptidase domain 19 (meltrin beta) (ADAM19) gene, cullin 7 (CUL7) gene, thyroglobulin (TG) gene, programmed cell death 1 ligand 2 (PDCD1LG2) gene, glutamate receptor (ionotropic (N-methyl D-aspartate-like 1A (GRINL1A) gene, mahogunin (ring finger 1 (MGRN1) gene, syntrophin (beta 2 (dystrophin-associated protein A1 (59 kDa (basic component 2) (SNTB2) gene, cyclin-dependent kinase 5 (regulatory subunit 1 (p35) (CDK5R1) gene, glucosidase (alpha; acid (GAA) gene, katanin p60 subunit A-like 2 (KATNAL2) gene, carcinoembryonic antigen-related cell adhesion molecule 4 (CEACAM4) gene, zinc finger protein 335 (ZNF335) gene, aspartate beta-hydroxylase domain containing 2 (ASPHD2) gene, acidic repeat containing (ACRC) gene, butyrophilin-like 3/butyrophilin-like 8 (BTNL3, BTLN8) gene, Moloney leukemia virus 10 homolog (mouse) (MOV10) gene, mediator complex subunit 12-like (MED12L) gene, kelch-like 6 (Drosophila) (KLHL6) gene, PDZ and LIM domain 5 (PDLIM5) gene, UDP-N-acetyl-alpha-D-galactosamine:polypeptide N-acetylgalactosaminyltransferase 10 (GALNT10) gene, secernin 1 (SCRN1) gene, vesicular (overexpressed in cancer (prosurvival protein 1 (VOPP1, RP11-289I10.2) gene, FK506 binding protein 9, 63 kDa (FKBP9, FKBP9, FKBP9L, AC091812.2) gene, kinesin family member 27 (KIF27) gene, piwi-like 4 (Drosophila) (PIWIL4) gene, telomerase-associated protein 1 (TEP1) gene, GTP cyclohydrolase 1, (GCH1) gene, proline rich 11, (PRR11) gene, cadherin 2, type 1, N-cadherin (neuronal) (CDH2) gene, protein phosphatase 1B-like (FLJ40125, AC138534.1) (PPM1N) gene, related RAS viral (r-ras) oncogene homolog, (RRAS) gene, dolichyl-diphosphooligosaccharide-protein glycosyltransferase, (DDOST) gene, anterior pharynx defective 1 homolog A (C. elegans) (APH1A) gene, tubulin tyrosine ligase (TTL) gene, testis expressed 261, (TEX261) gene, coenzyme Q2 homolog, prenyltransferase (yeast) (COQ2) gene, FCH and double SH3 domains 1, (FCHSD1) gene, BCL2-antagonist/killer 1, (BAK1) gene, solute carrier family 25 (mitochondrial carrier; phosphate carrier) member 25, (SLC25A25) gene, RELT tumor necrosis factor receptor, (RELT) gene, acid phosphatase 2, lysosomal, (ACP2) gene, TBC1 domain family, member 2B, (TBC1D2B) gene, Fanconi anemia, complementation group A, (FANCA) gene, solute carrier family 39 (metal ion transporter) member 11, (SLC39A11) gene.

3. A method according to claim 1, comprising: comparing the level of at least one IRC marker transcript to the level of a corresponding IRC marker transcript, wherein the IRC marker transcript is selected from the group consisting of: (a) a polynucleotide comprising a nucleotide sequence that shares at least 80% (or at least 81% to at least 99% and all integer percentages in between) sequence identity with the sequence set forth in any one of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513 or 515, or a complement thereof; (b) a polynucleotide comprising a nucleotide sequence that encodes a polypeptide comprising the amino acid sequence set forth in any one of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514 or 516; (c) a polynucleotide comprising a nucleotide sequence that encodes a polypeptide that shares at least 80% (or at least 81% to at least 99% and all integer percentages in between) sequence similarity or identity with at least a portion of the sequence set forth in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514 or 516; (d) a polynucleotide expression product comprising a nucleotide sequence that hybridizes to the sequence of (a), (b), (c) or a complement thereof, under at least medium or high stringency conditions.

4. A method according to claim 1, comprising: comparing the level of at least one IRC marker polypeptide to the level of a corresponding IRC marker polypeptide, wherein the IRC marker polypeptide is selected from the group consisting of: (i) a polypeptide comprising the amino acid sequence set forth in any one of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514 or 516; and (ii) a polypeptide comprising an amino acid sequence that shares at least 80% (or at least 81% to at least 99% and all integer percentages in between) sequence similarity or identity with the sequence set forth in any one of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514 or 516.

5. A method according to any one of claims 1 to 4, comprising: (1) measuring in a biological sample obtained from the subject the level of the at least one IRC marker expression product and (2) comparing the measured level of each IRC marker expression product to the level of a corresponding IRC marker expression product in a reference sample obtained from the at least one control subject.

6. A method according to any one of claims 1 to 5, comprising: assessing whether the subject has, or is at risk of developing, one of the plurality of conditions when the measured level of the or each IRC marker expression product is different than the measured level of the or each corresponding IRC marker expression product.

7. A method according to claim 6, wherein the level of an individual IRC marker expression product is at least 110% of the level of an individual corresponding IRC expression product.

8. A method according to claim 6, wherein the level of an individual IRC marker expression product is no more than about 95% of the level of an individual corresponding IRC expression product.

9. A method according to any one of claims 1 to 6 or 8, wherein the presence or risk of development of sepsis is determined by detecting in the subject a decrease in the level of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 IRC marker expression products from a multi-transcript-producing gene selected from the group consisting of KIF27, OTX1, CDK5R1, FKBP9, CDH2, ADAM19, BTNL8/3 and PANX2 (hereafter referred to as “LIST A”), as compared to the level of a corresponding IRC marker expression product(s) in a post-surgical inflammation-positive control subject.

10. A method according to any one of claims 1 to 7, wherein the presence or risk of development of post-surgical inflammation is determined by detecting in the subject an increase in the level of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48 IRC marker expression product(s) from at least one multi-transcript-producing gene selected from the group consisting of: KIF27, OTX1, CDK5R1, FKBP9, CDH2, ADAM19, BTNL8/3 and PANX2 (i.e., LIST A), as compared to the level of a corresponding IRC marker expression product in a sepsis control subject.

11. A method according to claim 9 or claim 10, wherein the KIF27 IRC marker expression product comprises a nucleotide sequence corresponding to KIF27 exons 4 and 7 or an amino acid sequence encoded by that exon.

12. A method according to claim 11, wherein the KIF27 IRC marker expression product is a KIF27 IRC marker transcript as set forth in any one of SEQ ID NO: 1, 3, 5, 7, or 9.

13. A method according to claim 11, wherein the KIF271 IRC marker expression product is a KIF27 IRC marker polypeptide as set forth in any one of SEQ ID NO: 2, 4, 6, 8, or 10.

14. A method according to claim 9 or claim 10, wherein the OTX1 IRC marker expression product comprises a nucleotide sequence corresponding to OTX1 exon 5 or an amino acid sequence encoded by that exon.

15. A method according to claim 14, wherein the OTX1 IRC marker expression product is an OTX1 IRC marker transcript as set forth in any one of SEQ ID NO: 11 or 13.

16. A method according to claim 14, wherein the OTX1 IRC marker expression product is an OTX1 IRC marker polypeptide as set forth in any one of SEQ ID NO:12 or 14.

17. A method according to claim 9 or claim 10, wherein the CDK5R1 IRC marker expression product comprises a nucleotide sequence corresponding to CDK5R1 exon 2 or an amino acid sequence encoded by that exon.

18. A method according to claim 17, wherein the CDK5R1 IRC marker expression product is a CDK5R1 IRC marker transcript as set forth in any one of SEQ ID NO: 15.

19. A method according to claim 17, wherein the CDK5R1 IRC marker expression product is a CDK5R1 IRC marker polypeptide as set forth in any one of SEQ ID NO: 16.

20. A method according to claim 9 or claim 10, wherein the FKBP9 IRC marker expression product comprises a nucleotide sequence corresponding to FKBP9 exon 10 or an amino acid sequence encoded by that exon.

21. A method according to claim 20, wherein the IRC marker expression product is an FKBP9 IRC marker transcript as set forth in any one of SEQ ID NO: 17.

22. A method according to claim 20, wherein the FKBP9 IRC marker expression product is an FKBP9 IRC marker polypeptide as set forth in any one of SEQ ID NO: 18.

23. A method according to claim 9 or claim 10, wherein the CDH2 IRC marker expression product comprises a nucleotide sequence corresponding to CDH2 exon 10 or an amino acid sequence encoded by that exon.

24. A method according to claim 23, wherein the CDH2 IRC marker expression product is a CDH2 IRC marker transcript as set forth in any one of SEQ ID NO: 19 and 21.

25. A method according to claim 23, wherein the CDH2 IRC marker expression product is a CDH2 IRC marker polypeptide as set forth in any one of SEQ ID NO:19 and 21.

26. A method according to claim 9 or claim 10, wherein the ADAM19 IRC marker expression product comprises a nucleotide sequence corresponding to ADAM19 exon 10 or an amino acid sequence encoded by that exon.

27. A method according to claim 26, wherein the ADAM19 IRC marker expression product is an ADAM 19 IRC marker transcript as set forth in any one of SEQ NO: 23, 25, 27 and 29.

28. A method according to claim 26, wherein the ADAM19 IRC marker expression product is an ADAM19 IRC marker polypeptide as set forth in any one of SEQ ID NO:24, 26, 28 and 30.

29. A method according to claim 9 or claim 10, wherein the BTNL8/3 IRC marker expression product comprises a nucleotide sequence corresponding to BTNL8/3 exon 6 or an amino acid sequence encoded by that exon.

30. A method according to claim 29, wherein the BTNL8/3 IRC marker expression product is a BTNL8/3IRC marker transcript as set forth in any one of SEQ ID NO: 31, 33, 35, 37, 39 and 41.

31. A method according to claim 29, wherein the BTNL8/3 IRC marker expression product is a BTNL8/3 IRC marker polypeptide as set forth in any one of SEQ ID NO: 32, 34, 36, 38, 40 and 42.

32. A method according to claim 9 or claim 10, wherein the PANX2 IRC marker expression product comprises a nucleotide sequence corresponding to an exon selected from PANX2 exon 1 and exon 2, or an amino acid sequence encoded by that exon.

33. A method according to claim 32, wherein the PANX2 IRC marker expression product is a PANX2 IRC transcript as set forth in any one of SEQ ID NO: 43, 45 or 47.

34. A method according to claim 32, wherein the PANX2 IRC marker expression product is a PANX2 IRC polypeptide as set forth in any one of SEQ ID NO:44, 46 or 48.

35. A method according to any one of claims 1 to 7, wherein the presence or risk of development of sepsis is determined by detecting in the subject an increase in the level of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157 or 158 IRC marker expression product(s) from at least one multi-transcript-producing gene selected from the group consisting of: PDLIM5, SCRN1, ASPHD2, VOPP1, ACRC, GALNT10, AC1385341, MED12L, RHBDF2, KLHL6, TEP1, PIWIL6, PRR1, RRAS, TG, ANKDD1A, GABRR2, MOV10, SLAMF7, PDCDILG2 and GCH1 (hereafter referred to as “LIST B”), as compared to the level of a corresponding IRC marker expression product in a post-surgical-positive subject control subject.

36. A method according to any one of claims 1 to 6, or 8, wherein the presence or risk of development of post-surgical inflammation is determined by detecting in the subject a decrease in the level of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157 or 158 IRC marker expression product(s) from at least one multi-transcript-producing gene selected from the group consisting of PDLIM5, SCRN1, ASPHD2, VOPP1, ACRC, GALNT10, AC1385341, MED12L, RHBDF2, KLHL6, TEP1, PIWIL6, PRR1, RRAS, TG, ANKDD1A, GABRR2, MOV10, SLAMF7, PDCDILG2 and GCH1 (i.e., LIST B), as compared to the level of a corresponding IRC marker expression product in a sepsis control subject.

37. A method according to claim 35 or claim 36, wherein the PDLIM5 IRC marker expression product comprises a nucleotide sequence corresponding to PDLIM5 exon 5 or an amino acid sequence encoded by that exon.

38. A method according to claim 37, wherein the PDLIM5 IRC marker expression product is a PDLIM5 IRC transcript as set forth in any one of SEQ ID NO: 49.

39. A method according to claim 37, wherein the PDLIM5 IRC marker expression product is a PDLIM5 IRC polypeptide as set forth in any one of SEQ ID NO: 50.

40. A method according to claim 35 or claim 36, wherein the SCRN1 IRC marker expression product comprises a nucleotide sequence corresponding to SCRN1 exon 5 or an amino acid sequence encoded by that exon.

41. A method according to claim 40, wherein the SCRN1 IRC marker expression product is a SCRN1 IRC transcript as set forth in any one of SEQ ID NO: 51, 53, 55, 57, 59, 61 or 63.

42. A method according to claim 40, wherein the SCRN1 IRC marker expression product is a SCRN1 IRC polypeptide as set forth in any one of SEQ ID NO: 52, 54, 56, 58, 60, 62 or 64.

43. A method according to claim 35 or claim 36, wherein the ASPHD2 IRC marker expression product comprises a nucleotide sequence corresponding to ASPHD2 exon 4 or an amino acid sequence encoded by that exon.

44. A method according to claim 43, wherein the ASPHD2 IRC marker expression product is an ASPHD2 IRC transcript as set forth in any one of SEQ ID NO:65, 67 or 69.

45. A method according to claim 43, wherein the ASPHD2 IRC marker expression product is an ASPHD2 IRC polypeptide as set forth in any one of SEQ ID NO:66, 68 or 70.

46. A method according to claim 35 or claim 36, wherein the VOPP1 IRC marker expression product comprises a nucleotide sequence corresponding to VOPP1 exon 3 or an amino acid sequence encoded by that exon.

47. A method according to claim 46, wherein the VOPP1 IRC marker expression product is a VOPP1 IRC transcript as set forth in any one of SEQ ID NO: 71, 73, 75, 77, 79, 81, 83, 85, 87, 89, 91 or 93.

48. A method according to claim 46, wherein the VOPP1 IRC marker expression product is a VOPP1 IRC polypeptide as set forth in any one of SEQ ID NO: 72, 74, 76, 78, 80, 82, 84, 86, 88, 90, 92 or 94.

49. A method according to claim 35 or claim 36, wherein the ACRC IRC marker expression product comprises a nucleotide sequence corresponding to ACRC exons 3 and 5 or an amino acid sequence encoded by that exon.

50. A method according to claim 49, wherein the ACRC IRC marker expression product is an ACRC IRC transcript as set forth in any one of SEQ ID NO:95 or 97.

51. A method according to claim 49, wherein the ACRC IRC marker expression product is an ACRC IRC polypeptide as set forth in any one of SEQ ID NO:96 or 98.

52. A method according to claim 35 or claim 36, wherein the GALNT10 IRC marker expression product comprises a nucleotide sequence corresponding to GALNT10 exon 6 or an amino acid sequence encoded by that exon.

53. A method according to claim 52, wherein the GALNT10 IRC marker expression product is a GALNT10 IRC transcript as set forth in any one of SEQ ID NO:99 or 101.

54. A method according to claim 52, wherein the GALNT10 IRC marker expression product is a GALNT10 IRC polypeptide as set forth in any one of SEQ ID NO:100 or 102.

55. A method according to claim 35 or claim 36, wherein the PPM1N IRC marker expression product comprises a nucleotide sequence corresponding to PPM1N exon 3 or an amino acid sequence encoded by that exon.

56. A method according to claim 55, wherein the PPM1N IRC marker expression product is a PPM1N IRC transcript as set forth in any one of SEQ ID NO: 107, 109, 111, 113, 115, 117, 119, 121 or 123.

57. A method according to claim 55, wherein the PPM1N IRC marker expression product is a PPM1N IRC polypeptide as set forth in any one of SEQ ID NO: 108, 110, 112, 114, 116, 118, 120, 122, or 124.

58. A method according to claim 35 or claim 36, wherein the MED12L IRC marker expression product comprises a nucleotide sequence corresponding to MED12L exon 17 or an amino acid sequence encoded by that exon.

59. A method according to claim 58, wherein the MED12L IRC marker expression product is a MED12L IRC transcript as set forth in any one of SEQ ID NO: 125 or 127.

60. A method according to claim 58, wherein the MED12L IRC marker expression product is a MED12L IRC polypeptide as set forth in any one of SEQ ID NO:126 or 128.

61. A method according to claim 35 or claim 36, wherein the RHBDF2 IRC marker expression product comprises a nucleotide sequence corresponding to RHBDF2 exons 6, 9, 10, 11, 14, 17, 18 or 19 or an amino acid sequence encoded by that exon.

62. A method according to claim 61, wherein the RHBDF2 IRC marker expression product is an RHBDF2 IRC transcript as set forth in any one of SEQ ID NO: 129, 131 or 133.

63. A method according to claim 61, wherein the RHBDF2 IRC marker expression product is an RHBDF2 IRC polypeptide as set forth in any one of SEQ ID NO:130, 132 or 134.

64. A method according to claim 35 or claim 36, wherein the KLHL6 IRC marker expression product comprises a nucleotide sequence corresponding to KLHL6 exon 7 or an amino acid sequence encoded by that exon.

65. A method according to claim 64, wherein the KLHL6 IRC marker expression product is a KLHL6 IRC transcript as set forth in any one of SEQ ID NO: 135.

66. A method according to claim 64, wherein the KLHL6 IRC marker expression product is a KLHL6 IRC polypeptide as set forth in any one of SEQ ID NO:136.

67. A method according to claim 35 or claim 36, wherein the TEP1 IRC marker expression product comprises a nucleotide sequence corresponding to TEP1 exon 49 or an amino acid sequence encoded by that exon.

68. A method according to claim 67, wherein the TEP1 IRC marker expression product is a TEP1 IRC transcript as set forth in any one of SEQ ID NO: 137 or 139.

69. A method according to claim 67, wherein the TEP1 IRC marker expression product is a TEP1 IRC polypeptide as set forth in any one of SEQ ID NO:138 or 140.

70. A method according to claim 35 or claim 36, wherein the PIWIL4 IRC marker expression product comprises a nucleotide sequence corresponding to PIWIL4 exons 2 and 14 or an amino acid sequence encoded by that exon.

71. A method according to claim 70, wherein the PIWIL4 IRC marker expression product is a PIWIL4 IRC transcript as set forth in any one of SEQ ID NO: 141 or 143.

72. A method according to claim 70, wherein the PIWIL4 IRC marker expression product is a PIWIL4 IRC polypeptide as set forth in any one of SEQ ID NO:142 or 144.

73. A method according to claim 35 or claim 36, wherein the PRR11 IRC marker expression product comprises a nucleotide sequence corresponding to PRR11 exons 4 and 5 or an amino acid sequence encoded by that exon.

74. A method according to claim 73, wherein the PRR11 IRC marker expression product is a PRR11 IRC transcript as set forth in any one of SEQ ID NO: 145.

75. A method according to claim 73, wherein the PRR11 IRC marker expression product is a PRR11 IRC polypeptide as set forth in any one of SEQ ID NO:146.

76. A method according to claim 35 or claim 36, wherein the RRAS IRC marker expression product comprises a nucleotide sequence corresponding to RRAS exon 1 or an amino acid sequence encoded by that exon.

77. A method according to claim 76, wherein the RRAS IRC marker expression product is an RRAS IRC transcript as set forth in any one of SEQ ID NO: 147.

78. A method according to claim 76, wherein the RRAS IRC marker expression product is an RRAS IRC polypeptide as set forth in any one of SEQ ID NO:148.

79. A method according to claim 35 or claim 36, wherein the TG IRC marker expression product comprises a nucleotide sequence corresponding to TG exon 6 or an amino acid sequence encoded by that exon.

80. A method according to claim 79, wherein the TG IRC marker expression product is a TG IRC transcript as set forth in any one of SEQ ID NO: 149 or 151.

81. A method according to claim 79, wherein the TG IRC marker expression product is a TG IRC polypeptide as set forth in any one of SEQ ID NO:150 or 152.

82. A method according to claim 35 or claim 36, wherein the ANKDD1A IRC marker expression product comprises a nucleotide sequence corresponding to ANKDD1A exon 7 or an amino acid sequence encoded by that exon.

83. A method according to claim 82, wherein the ANKDD1A IRC marker expression product is an ANKDD1A IRC transcript as set forth in any one of SEQ ID NO: 153, 155, 157, 159 or 161.

84. A method according to claim 82, wherein the ANKDD1A IRC marker expression product is an ANKDD1A IRC polypeptide as set forth in any one of SEQ ID NO:154, 156, 158, 160 or 162.

85. A method according to claim 35 or claim 36, wherein the GABRR2 IRC marker expression product comprises a nucleotide sequence corresponding to GABRR2 exons 7, 8 or 9 or an amino acid sequence encoded by that exon.

86. A method according to claim 85, wherein the GABRR2 IRC marker expression product is an GABRR2 IRC transcript as set forth in any one of SEQ ID NO: 163 or 165.

87. A method according to claim 85, wherein the GABRR2 IRC marker expression product is an GABRR2 IRC polypeptide as set forth in any one of SEQ ID NO:164 or 166.

88. A method according to claim 35 or claim 36, wherein the MOV10 IRC marker expression product comprises a nucleotide sequence corresponding to MOV10 exon 6 or an amino acid sequence encoded by that exon.

89. A method according to claim 88, wherein the MOV10 IRC marker expression product is a MOV10 IRC transcript as set forth in any one of SEQ ID NO: 167, 169, 171, 173, 175 or 177.

90. A method according to claim 85, wherein the MOV10 IRC marker expression product is a MOV10 IRC polypeptide as set forth in any one of SEQ ID NO:168, 170, 172, 174, 176 or 178.

91. A method according to claim 35 or claim 36, wherein the SLAMF7 IRC marker expression product comprises a nucleotide sequence corresponding to SLAMF7 exons 2, 3, 4 or 5 or an amino acid sequence encoded by that exon.

92. A method according to claim 91, wherein the SLAMF7 IRC marker expression product is a SLAMF7 IRC transcript as set forth in any one of SEQ ID NO: 179, 181, 183, 185, 187, 189, 191 or 193.

93. A method according to claim 91, wherein the SLAMF7 IRC marker expression product is a SLAMF7 IRC polypeptide as set forth in any one of SEQ ID NO:180, 182, 184, 186, 188, 190, 192 or 194.

94. A method according to claim 35 or claim 36, wherein the PDCD1LG2 IRC marker expression product comprises a nucleotide sequence corresponding to PDCD1LG2 exons 1 or 2 or an amino acid sequence encoded by that exon.

95. A method according to claim 94, wherein the PDCD1LG2 IRC marker expression product is a PDCD1LG2 IRC transcript as set forth in any one of SEQ ID NO:195 or 197.

96. A method according to claim 94, wherein the PDCD1LG2 IRC marker expression product is a PDCD1LG2 IRC polypeptide as set forth in any one of SEQ ID NO: 196 or 198.

97. A method according to claim 35 or claim 36, wherein the GCH1 RC marker expression product comprises a nucleotide sequence corresponding to GCH1 exon 2 or an amino acid sequence encoded by that exon.

98. A method according to claim 97, wherein the GCH1 IRC marker expression product is a GCH1 IRC transcript as set forth in any one of SEQ ID NO:199, 201, 203 or 205.

99. A method according to claim 97, wherein the GCH1 IRC marker expression product is a GCH1 IRC polypeptide as set forth in any one of SEQ ID NO: 200, 202, 204 or 206.

100. A method according to any one of claims 1 to 7, wherein the presence or risk of development of sepsis is determined by detecting in the subject an increase in the level of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155 or 156 IRC marker expression product(s) from at least one multi-transcript-producing gene selected from the group consisting of: RELT, ACP2, FCHSD1, CLPB, SLC39A1, TBC1D2B, APH1A, DDOST, BAK1, SLC25A25A, COQ2, FANCA, PIWIL4, ZNF335, TEX261, GABRR2, VOPP1, TTL, CES2, GALNT10, CQORF91, AMBRA1 and SCRN1 (hereafter referred to as “LIST C”), as compared to the level of a corresponding IRC marker expression product in an inSIRS-positive control subject.

101. A method according to any one of claims 1 to 6, or 8, wherein the presence or risk of development of inSIRS is determined by detecting in the subject a decrease in the level of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155 or 156 IRC marker expression(s) product from at least one multi-transcript-producing gene selected from the group consisting of: RELT, ACP2, FCHSD1, CLPB, SLC39A1, TBC1D2B, APH1A, DDOST, BAK1, SLC25A25A, COQ2, FANCA, PIWIL4, ZNF335, TEX261, GABRR2, VOPP1, TTL, CES2, GALNT10, CQORF91, AMBRA1 and SCRN1 (i.e., LIST C), as compared to the level of the corresponding IRC marker expression product in a sepsis-positive control subject.

102. A method according to claim 100 or claim 101, wherein the RELT IRC marker expression product comprises a nucleotide sequence corresponding to an exon selected from RELT exon 4, or an amino acid sequence encoded by that exon.

103. A method according to claim 102, wherein the RELT IRC marker expression product is a RELT IRC transcript as set forth in any one of SEQ ID NO: 207 or 209.

104. A method according to claim 102, wherein the RELT IRC marker expression product is a RELT IRC polypeptide as set forth in any one of SEQ ID NO: 208 or 210.

105. A method according to claim 100 or claim 101, wherein the ACP2 IRC marker expression product comprises a nucleotide sequence corresponding to an exon selected from ACP2 exon 7, or an amino acid sequence encoded by that exon.

106. A method according to claim 105, wherein the ACP2 IRC marker expression product is a ACP2 IRC transcript as set forth in any one of SEQ ID NO: 211.

107. A method according to claim 105, wherein the ACP2 IRC marker expression product is a ACP2 IRC polypeptide as set forth in any one of SEQ ID NO: 212.

108. A method according to claim 100 or claim 101, wherein the FCHSD1 IRC marker expression product comprises a nucleotide sequence corresponding to an exon selected from FCHSD1 exon 14, or an amino acid sequence encoded by that exon.

109. A method according to claim 108, wherein the FCHSD1 IRC marker expression product is a FCHSD1 IRC transcript as set forth in any one of SEQ ID NO: 213 or 215.

110. A method according to claim 108, wherein the FCHSD1 IRC marker expression product is a FCHSD1 IRC polypeptide as set forth in any one of SEQ ID NO: 214 or 216.

111. A method according to claim 100 or claim 101, wherein the CLPB IRC marker expression product comprises a nucleotide sequence corresponding to an exon selected from CLPB exon 10, or an amino acid sequence encoded by that exon.

112. A method according to claim 111, wherein the CLPB IRC marker expression product is a CLPB IRC transcript as set forth in any one of SEQ ID NO: 217, 219 or 221.

113. A method according to claim 111, wherein the CLPB IRC marker expression product is a CLPB IRC polypeptide as set forth in any one of SEQ ID NO: 218, 220 or 222.

114. A method according to claim 100 or claim 101, wherein the SLC39A11 IRC marker expression product comprises a nucleotide sequence corresponding to an exon selected from SLC39A11 exon 2, or an amino acid sequence encoded by that exon.

115. A method according to claim 114, wherein the SLC39A11 IRC marker expression product is a SLC39A11 IRC transcript as set forth in any one of SEQ ID NO: 223.

116. A method according to claim 114, wherein the SLC39A11 IRC marker expression product is a SLC39A11 IRC polypeptide as set forth in any one of SEQ ID NO: 224.

117. A method according to claim 100 or claim 101, wherein the TBC1D2B IRC marker expression product comprises a nucleotide sequence corresponding to an exon selected from TBC1D2B exon 13, or an amino acid sequence encoded by that exon.

118. A method according to claim 117, wherein the TBC1D2B IRC marker expression product is a TBC1D2B IRC transcript as set forth in any one of SEQ ID NO: 225, 227 or 229.

119. A method according to claim 117, wherein the TBC1D2B IRC marker expression product is a TBC1D2B IRC polypeptide as set forth in any one of SEQ ID NO: 226, 228 or 230.

120. A method according to claim 100 or claim 101, wherein the APH1A IRC marker expression product comprises a nucleotide sequence corresponding to an exon selected from APH1A exon 1, or an amino acid sequence encoded by that exon.

121. A method according to claim 120, wherein the APH1A IRC marker expression product is an APH1A IRC transcript as set forth in any one of SEQ ID NO: 231, 233, 235, 237, 239 or 241.

122. A method according to claim 120, wherein the APH1A IRC marker expression product is a APH1A IRC polypeptide as set forth in any one of SEQ ID NO: 232, 234, 236, 238, 240 or 242.

123. A method according to claim 100 or claim 101, wherein the DDOST IRC marker expression product comprises a nucleotide sequence corresponding to an exon selected from DDOST exon 2, or an amino acid sequence encoded by that exon.

124. A method according to claim 123, wherein the DDOST IRC marker expression product is a DDOST IRC transcript as set forth in any one of SEQ ID NO: 243.

125. A method according to claim 123, wherein the DDOST IRC marker expression product is a DDOST IRC polypeptide as set forth in any one of SEQ ID NO: 244.

126. A method according to claim 100 or claim 101, wherein the BAK1 IRC marker expression product comprises a nucleotide sequence corresponding to an exon selected from BAK1 exon 7, or an amino acid sequence encoded by that exon.

127. A method according to claim 126, wherein the BAK1 IRC marker expression product is a BAK1 IRC transcript as set forth in any one of SEQ ID NO: 245 or 247.

128. A method according to claim 126, wherein the BAK1 IRC marker expression product is a BAK1 IRC polypeptide as set forth in any one of SEQ ID NO: 246 or 248.

129. A method according to claim 100 or claim 101, wherein the SLC25A25A IRC marker expression product comprises a nucleotide sequence corresponding to an exon selected from SLC25A25A exon 10, or an amino acid sequence encoded by that exon.

130. A method according to claim 129, wherein the SLC25A25A IRC marker expression product is an SLC25A25A IRC transcript as set forth in any one of SEQ ID NO: 249, 251, 253, 255, 257, 259 or 261.

131. A method according to claim 129, wherein the SLC25A25A IRC marker expression product is an SLC25A25A IRC polypeptide as set forth in any one of SEQ ID NO: 250, 252, 254, 256, 258, 260 or 262.

132. A method according to claim 100 or claim 101, wherein the COQ2 IRC marker expression product comprises a nucleotide sequence corresponding to an exon selected from COQ2 exon 1, or an amino acid sequence encoded by that exon.

133. A method according to claim 132, wherein the COQ2 IRC marker expression product is a COQ2 IRC transcript as set forth in any one of SEQ ID NO: 263, 265 or 267.

134. A method according to claim 132, wherein the COQ2 IRC marker expression product is a COQ2 IRC polypeptide as set forth in any one of SEQ ID NO: 264, 266 or 268.

135. A method according to claim 100 or claim 101, wherein the FANCA IRC marker expression product comprises a nucleotide sequence corresponding to an exon selected from FANCA exon 35, or an amino acid sequence encoded by that exon.

136. A method according to claim 135, wherein the FANCA IRC marker expression product is a FANCA IRC transcript as set forth in any one of SEQ ID NO: 269 or 271.

137. A method according to claim 135, wherein the FANCA IRC marker expression product is a FANCA IRC polypeptide as set forth in any one of SEQ ID NO: 270 or 272.

138. A method according to claim 100 or claim 101, wherein the PIWIL4 IRC marker expression product comprises a nucleotide sequence corresponding to an exon selected from PIWIL4 exons 2, 14, or an amino acid sequence encoded by that exon.

139. A method according to claim 138, wherein the PIWIL4 IRC marker expression product is a PIWIL4 IRC transcript as set forth in any one of SEQ ID NO: 273 or 275.

140. A method according to claim 138, wherein the PIWIL4 IRC marker expression product is a PIWIL4 IRC polypeptide as set forth in any one of SEQ ID NO: 274 or 276.

141. A method according to claim 100 or claim 101, wherein the ZNF335 IRC marker expression product comprises a nucleotide sequence corresponding to an exon selected from ZNF335 exon 5, or an amino acid sequence encoded by that exon.

142. A method according to claim 141, wherein the ZNF335 IRC marker expression product is a ZNF335 IRC transcript as set forth in any one of SEQ ID NO: 277, 279 or 281.

143. A method according to claim 141, wherein the ZNF335 IRC marker expression product is a ZNF335 IRC polypeptide as set forth in any one of SEQ ID NO: 278, 280 or 282.

144. A method according to claim 100 or claim 101, wherein the TEX261 IRC marker expression product comprises a nucleotide sequence corresponding to an exon selected from TEX261 exon 3, or an amino acid sequence encoded by that exon.

145. A method according to claim 144, wherein the TEX261 IRC marker expression product is a TEX261 IRC transcript as set forth in any one of SEQ ID NO: 283 or 285.

146. A method according to claim 144, wherein the TEX261 IRC marker expression product is a TEX261 IRC polypeptide as set forth in any one of SEQ ID NO: 284 or 286.

147. A method according to claim 100 or claim 101, wherein the GABRR2 IRC marker expression product comprises a nucleotide sequence corresponding to an exon selected from GABRR2 exons 7, 8, 9 or an amino acid sequence encoded by that exon.

148. A method according to claim 147, wherein the GABRR2 IRC marker expression product is a GABRR2 IRC transcript as set forth in any one of SEQ ID NO: 287 or 289.

149. A method according to claim 147, wherein the GABRR2 IRC marker expression product is a GABRR2 IRC polypeptide as set forth in any one of SEQ ID NO: 288 or 290.

150. A method according to claim 100 or claim 101, wherein the VOPP1 IRC marker expression product comprises a nucleotide sequence corresponding to an exon selected from VOPP1 exon 3 or an amino acid sequence encoded by that exon.

151. A method according to claim 150, wherein the VOPP1 IRC marker expression product is a VOPP1 IRC transcript as set forth in any one of SEQ ID NO: 291, 293, 295, 297, 299, 301, 303, 305, 307, 309, 311 or 313.

152. A method according to claim 150, wherein the VOPP1 IRC marker expression product is a VOPP1 IRC polypeptide as set forth in any one of SEQ ID NO: 292, 294, 296, 298, 300, 302, 304, 306, 308, 310, 312 or 314.

153. A method according to claim 100 or claim 101, wherein the TTL IRC marker expression product comprises a nucleotide sequence corresponding to an exon selected from TTL exon 7 or an amino acid sequence encoded by that exon.

154. A method according to claim 153, wherein the TTL IRC marker expression product is a TTL IRC transcript as set forth in any one of SEQ ID NO: 315.

155. A method according to claim 153, wherein the TTL IRC marker expression product is a TTL IRC polypeptide as set forth in any one of SEQ ID NO: 316.

156. A method according to claim 100 or claim 101, wherein the CES2 IRC marker expression product comprises a nucleotide sequence corresponding to an exon selected from CES2 exon 1 or an amino acid sequence encoded by that exon.

157. A method according to claim 156, wherein the CES2 IRC marker expression product is a CES2 IRC transcript as set forth in any one of SEQ ID NO: 317 or 319.

158. A method according to claim 156, wherein the CES2 IRC marker expression product is a CES2 IRC polypeptide as set forth in any one of SEQ ID NO: 318 or 320.

159. A method according to claim 100 or claim 101, wherein the GALNT10 IRC marker expression product comprises a nucleotide sequence corresponding to an exon selected from GALNT10 exon 6 or an amino acid sequence encoded by that exon.

160. A method according to claim 159, wherein the GALNT10 IRC marker expression product is a GALNT10 IRC transcript as set forth in any one of SEQ ID NO: 321 or 323.

161. A method according to claim 159, wherein the GALNT10 IRC marker expression product is a GALNT10 IRC polypeptide as set forth in any one of SEQ ID NO: 322 or 324.

162. A method according to claim 100 or claim 101, wherein the C1orf91 IRC marker expression product comprises a nucleotide sequence corresponding to an exon selected from C1orf91 exon 2 or an amino acid sequence encoded by that exon.

163. A method according to claim 162, wherein the C1orf91 IRC marker expression product is a C1orf91 IRC transcript as set forth in any one of SEQ ID NO: 325, 327, 329, 331, 333 or 335.

164. A method according to claim 162, wherein the C1orf91 IRC marker expression product is a C1orf91 IRC polypeptide as set forth in any one of SEQ ID NO: 326, 328, 330, 332, 334 or 336.

165. A method according to claim 100 or claim 101, wherein the AMBRA1 IRC marker expression product comprises a nucleotide sequence corresponding to an exon selected from AMBRA1 exons 2, 4 or an amino acid sequence encoded by that exon.

166. A method according to claim 165, wherein the AMBRA1 IRC marker expression product is a AMBRA1 IRC transcript as set forth in any one of SEQ ID NO: 337, 339, 341, 343, 345 or 347.

167. A method according to claim 165, wherein the AMBRA1 IRC marker expression product is a AMBRA1 IRC polypeptide as set forth in any one of SEQ ID NO: 338, 340, 342, 344, 346 or 348.

168. A method according to claim 100 or claim 101, wherein the SCRN IRC marker expression product comprises a nucleotide sequence corresponding to an exon selected from SCRN exon 5 or an amino acid sequence encoded by that exon.

169. A method according to claim 168, wherein the SCRN IRC marker expression product is a SCRN IRC transcript as set forth in any one of SEQ ID NO: 349, 3512, 353, 355, 357, 359 or 361.

170. A method according to claim 168, wherein the SCRN IRC marker expression product is a SCRN IRC polypeptide as set forth in any one of SEQ ID NO: 350, 352, 354, 356, 358, 360 or 362.

171. A method according to any one of claims 1 to 7, wherein the presence or risk of development of inSIRS is determined by detecting in the subject a increase in the level of at least 1, 2, 3, 4, 5, 6, 7, 8, 9, 10, 11, 12, 13, 14, 15, 16, 17, 18, 19 or 20 IRC marker expression(s) product from at least one multi-transcript-producing gene selected from the group consisting of: GRINL1A and KATLNAL2 (i.e., LIST D), as compared to the level of the corresponding IRC marker expression product in a sepsis-positive control subject.

172. A method according to any one of claims 1 to 6, or 8, wherein the presence or risk of development of sepsis is determined by detecting in the subject an decrease in the level of at least 1, 2, 3, 4, 5, 6, 7, 8, 9, 10, 11, 12, 13, 14, 15, 16, 17, 18, 19 or 20 IRC marker expression product(s) from at least one multi-transcript-producing gene selected from the group consisting of GRINL1A and KATLNAL2 (hereafter referred to as “LIST D”), as compared to the level of a corresponding IRC marker expression product in an inSIRS-positive control subject.

173. A method according to claim 171 or claim 172, wherein the GRINL1A marker expression product comprises a nucleotide sequence corresponding to an exon selected from GRINL1A exon 5 or an amino acid sequence encoded by that exon.

174. A method according to claim 173, wherein the GRINL1A IRC marker expression product is a GRINL1A IRC transcript as set forth in any one of SEQ ID NO: 363, 365, 367, 369, 371, 373, 375 or 377.

175. A method according to claim 173, wherein the GRINL1A IRC marker expression product is a GRINL1A IRC polypeptide as set forth in any one of SEQ ID NO: 364, 366, 368, 370, 372, 374, 376 or 378.

176. A method according to claim 171 or claim 172, wherein the KATNAL2 marker expression product comprises a nucleotide sequence corresponding to an exon selected from KATNAL2 exon 3 or an amino acid sequence encoded by that exon.

177. A method according to claim 176, wherein the KATNAL2 IRC marker expression product is a KATNAL2 IRC transcript as set forth in any one of SEQ ID NO: 379 or 381.

178. A method according to claim 176, wherein the KATNAL2 IRC marker expression product is a KATNAL2 IRC polypeptide as set forth in any one of SEQ ID NO: 380 or 382.

179. A method according to any one of claims 1 to 7, wherein the presence or risk of development of inSIRS is determined by detecting in the subject an increase in the level of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 IRC marker expression products) from at least one multi-transcript-producing gene selected from the group consisting of: KATLNAL2, GRINL1A, ACRC, TG and ASPHD2 (hereafter referred to as “LIST E”), as compared to the level of a corresponding IRC marker expression product in an post-surgical inflammation-positive control subject.

180. A method according to any one of claims 1 to 6, or 8, wherein the presence or risk of development of post-surgical inflammation is determined by detecting in the subject a decrease in the level of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 IRC marker expression(s) product from at least one multi-transcript-producing gene selected from the group consisting of: KATLNAL2, GRINL1A, ACRC, TG and ASPHD2 (i.e., LIST E), as compared to the level of the corresponding IRC marker expression product in a inSIRS-positive control subject.

181. A method according to claim 179 or claim 180, wherein the KATLNAL2 IRC marker expression product comprises a nucleotide sequence corresponding to an exon selected from KATLNAL2 exon 3, or an amino acid sequence encoded by that exon.

182. A method according to claim 181, wherein the KATLNAL2 IRC marker expression product is a KATLNAL2 IRC transcript as set forth in any one of SEQ ID NO: 387 or 389.

183. A method according to claim 181, wherein the KATLNAL2 IRC marker expression product is a KATLNAL2 IRC polypeptide as set forth in any one of SEQ ID NO: 388 or 390.

184. A method according to claim 179 or claim 180, wherein the GRINL1A IRC marker expression product comprises a nucleotide sequence corresponding to an exon selected from GRINL1A exon 5, or an amino acid sequence encoded by that exon.

185. A method according to claim 184, wherein the GRINL1A IRC marker expression product is a GRINL1A IRC transcript as set forth in any one of SEQ ID NO: 391, 393, 395, 397, 399, 401, 403 or 405.

186. A method according to claim 184, wherein the GRINL1A IRC marker expression product is a GRINL1 A IRC polypeptide as set forth in any one of SEQ ID NO: 392, 394, 396, 398, 400, 402, 404 or 406.

187. A method according to claim 179 or claim 180, wherein the ACRC IRC marker expression product comprises a nucleotide sequence corresponding to an exon selected from ACRC exon 3, 5 or an amino acid sequence encoded by that exon.

188. A method according to claim 187, wherein the ACRC IRC marker expression product is a ACRC IRC transcript as set forth in any one of SEQ ID NO:407 or 409.

189. A method according to claim 187, wherein the ACRC IRC marker expression product is a ACRC IRC polypeptide as set forth in any one of SEQ ID NO: 408 or 410.

190. A method according to claim 179 or claim 180, wherein the TG IRC marker expression product comprises a nucleotide sequence corresponding to an exon selected from TG exon 6 or an amino acid sequence encoded by that exon.

191. A method according to claim 190, wherein the TG IRC marker expression product is a TG IRC transcript as set forth in any one of SEQ ID NO:411 or 413.

192. A method according to claim 190, wherein the TG IRC marker expression product is a TG IRC polypeptide as set forth in any one of SEQ ID NO: 412 or 414.

193. A method according to claim 179 or claim 180, wherein the ASPHD2 IRC marker expression product comprises a nucleotide sequence corresponding to an exon selected from ASPHD2 exon 4 or an amino acid sequence encoded by that exon.

194. A method according to claim 193, wherein the ASPHD2 IRC marker expression product is an ASPHD2 IRC transcript as set forth in any one of SEQ ID NO:415, 417 or 419.

195. A method according to claim 193, wherein the ASPHD2 IRC marker expression product is an ASPHD2 IRC polypeptide as set forth in any one of SEQ ID NO: 416, 418 or 420.

196. A method according to any one of claims 1 to 7, wherein the presence or risk of development of post-surgical inflammation is determined by detecting in the subject an increase in the level of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95 or 96 IRC marker expression(s) product from at least one multi-transcript-producing gene selected from the group consisting of: CUL7, BTNL8/3, PANX2, C1orf91, ZNF335, MGRN1, GAA, CDK5R1, SNTB2, CLPB, ADMA19, SLC36A1, FKBP9, NDST1, HIPK2 and CEACAM4 (i.e., LIST F), as compared to the level of the corresponding IRC marker expression product in a inSIRS-positive control subject.

197. A method according to any one of claims 1 to 6, or 8, wherein the presence or risk of development of inSIRS is determined by detecting in the subject a decrease in the level of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95 or 96 IRC marker expression product(s) from at least one multi-transcript-producing gene selected from the group consisting of CUL7, BTNL8/3, PANX2, C1orf91, ZNF335, MGRN1, GAA, CDK5R1, SNTB2, CLPB, ADMA19, SLC36A1, FKBP9, NDST1, HIPK2 and CEACAM4 (hereafter referred to as “LIST F”), as compared to the level of a corresponding IRC marker expression product in an post-surgical inflammation-positive control subject.

198. A method according to claim 196 or claim 197, wherein the CUL7 IRC marker expression product comprises a nucleotide sequence corresponding to an exon selected from CUL7 exon 5 or an amino acid sequence encoded by that exon.

199. A method according to claim 198, wherein the CUL7 IRC marker expression product is a CUL7 IRC transcript as set forth in any one of SEQ ID NO: 421.

200. A method according to claim 198, wherein the CUL7 IRC marker expression product is a CUL7 IRC polypeptide as set forth in any one of SEQ ID NO: 422.

201. A method according to claim 196 or claim 197, wherein the BTNL8/3 IRC marker expression product comprises a nucleotide sequence corresponding to an exon selected from BTNL8/3 exon 6 or an amino acid sequence encoded by that exon.

202. A method according to claim 201, wherein the BTNL8 IRC marker expression product is a BTNL8/3 IRC transcript as set forth in any one of SEQ ID NO: 423, 425, 427, 429, 431 or 433.

203. A method according to claim 201, wherein the BTNL8 IRC marker expression product is a BTNL8/3 IRC polypeptide as set forth in any one of SEQ ID NO: 424, 426, 428, 430, 432 or 434.

204. A method according to claim 196 or claim 197, wherein the PANX2 IRC marker expression product comprises a nucleotide sequence corresponding to an exon selected from PANX2 exon 1, 2 or an amino acid sequence encoded by that exon.

205. A method according to claim 204, wherein the PANX2 IRC marker expression product is a PANX2 IRC transcript as set forth in any one of SEQ ID NO: 435, 437 or 439.

206. A method according to claim 204, wherein the PANX2 IRC marker expression product is a PANX2 IRC polypeptide as set forth in any one of SEQ ID NO: 436, 438 or 440.

207. A method according to claim 196 or claim 197, wherein the C1orf91 IRC marker expression product comprises a nucleotide sequence corresponding to an exon selected from C1orf91 exon 2 or an amino acid sequence encoded by that exon.

208. A method according to claim 207, wherein the C1orf91 IRC marker expression product is a C1orf91 IRC transcript as set forth in any one of SEQ ID NO: 441, 443, 445, 447, 449 or 451.

209. A method according to claim 207, wherein the C1orf91 IRC marker expression product is a C1orf91 IRC polypeptide as set forth in any one of SEQ ID NO: 442, 444, 446, 448, 450 or 452.

210. A method according to claim 196 or claim 197, wherein the ZNF335 IRC marker expression product comprises a nucleotide sequence corresponding to an exon selected from ZNF335 exon 5 or an amino acid sequence encoded by that exon.

211. A method according to claim 210, wherein the ZNF335 IRC marker expression product is a ZNF335 IRC transcript as set forth in any one of SEQ ID NO: 453, 455 or 457.

212. A method according to claim 210, wherein the ZNF335 IRC marker expression product is a ZNF335 IRC polypeptide as set forth in any one of SEQ ID NO: 454, 456 or 458.

213. A method according to claim 196 or claim 197, wherein the MGRN1 IRC marker expression product comprises a nucleotide sequence corresponding to an exon selected from MGRN1 exon 4 or an amino acid sequence encoded by that exon.

214. A method according to claim 213, wherein the MGRN1 IRC marker expression product is a MGRN1 IRC transcript as set forth in any one of SEQ ID NO: 459, 461 or 463.

215. A method according to claim 213, wherein the MGRN1 IRC marker expression product is a MGRN1 IRC polypeptide as set forth in any one of SEQ ID NO:460, 462 or 464.

216. A method according to claim 196 or claim 197, wherein the GAA IRC marker expression product comprises a nucleotide sequence corresponding to an exon selected from GAA exon 3 or an amino acid sequence encoded by that exon.

217. A method according to claim 216, wherein the GAA IRC marker expression product is a GAA IRC transcript as set forth in any one of SEQ ID NO: 465, 467 or 469.

218. A method according to claim 216, wherein the GAA IRC marker expression product is a GAA IRC polypeptide as set forth in any one of SEQ ID NO:466, 468 or 470.

219. A method according to claim 196 or claim 197, wherein the CDK5R1 IRC marker expression product comprises a nucleotide sequence corresponding to an exon selected from CDK5R1 exon 2 or an amino acid sequence encoded by that exon.

220. A method according to claim 219, wherein the CDK5R1 IRC marker expression product is a CDK5R1 IRC transcript as set forth in any one of SEQ ID NO: 471.

221. A method according to claim 219, wherein the CDK5R1 IRC marker expression product is a CDK5R1 IRC polypeptide as set forth in any one of SEQ ID NO: 472.

222. A method according to claim 196 or claim 197, wherein the SNTB2 IRC marker expression product comprises a nucleotide sequence corresponding to an exon selected from SNTB2 exon 4 or an amino acid sequence encoded by that exon.

223. A method according to claim 222, wherein the SNTB2 IRC marker expression product is a SNTB2 IRC transcript as set forth in any one of SEQ ID NO: 473.

224. A method according to claim 222, wherein the SNTB2 IRC marker expression product is a SNTB2 IRC polypeptide as set forth in any one of SEQ ID NO: 474.

225. A method according to claim 196 or claim 197, wherein the CLPB IRC marker expression product comprises a nucleotide sequence corresponding to an exon selected from CLPB exon 10 or an amino acid sequence encoded by that exon.

226. A method according to claim 225, wherein the CLPB IRC marker expression product is a CLPB IRC transcript as set forth in any one of SEQ ID NO: 475, 477 or 479.

227. A method according to claim 225, wherein the CLPB IRC marker expression product is a CLPB IRC polypeptide as set forth in any one of SEQ ID NO: 478 or 480.

228. A method according to claim 196 or claim 197, wherein the ADAM19 IRC marker expression product comprises a nucleotide sequence corresponding to an exon selected from ADAM19 exon 10 or an amino acid sequence encoded by that exon.

229. A method according to claim 228, wherein the ADAM19 IRC marker expression product is an ADAM19 IRC transcript as set forth in any one of SEQ ID NO: 481, 483, 485 or 487.

230. A method according to claim 228, wherein the ADAM19 IRC marker expression product is an ADAM19 IRC polypeptide as set forth in any one of SEQ ID NO: 482, 484, 486 or 488.

231. A method according to claim 196 or claim 197, wherein the SLC36A1 IRC marker expression product comprises a nucleotide sequence corresponding to an exon selected from SLC36A1 exon 5 or an amino acid sequence encoded by that exon.

232. A method according to claim 231, wherein the SLC36A1 IRC marker expression product is a SLC36A1 IRC transcript as set forth in any one of SEQ ID NO: 489, 491, 493 or 495.

233. A method according to claim 231, wherein the SLC36A1 IRC marker expression product is a SLC36A1 IRC polypeptide as set forth in any one of SEQ ID NO: 490, 492, 494 or 496.

234. A method according to claim 196 or claim 197, wherein the FKBP9 IRC marker expression product comprises a nucleotide sequence corresponding to an exon selected from FKBP9 exon 10 or an amino acid sequence encoded by that exon.

235. A method according to claim 234, wherein the FKBP9 IRC marker expression product is a FKBP9 IRC transcript as set forth in any one of SEQ ID NO: 497 or 499.

236. A method according to claim 234, wherein the FKBP9 IRC marker expression product is a FKBP9 IRC polypeptide as set forth in any one of SEQ ID NO: 498 or 500.

237. A method according to claim 196 or claim 197, wherein the NDST1 IRC marker expression product comprises a nucleotide sequence corresponding to an exon selected from NDST1 exon 3 or an amino acid sequence encoded by that exon.

238. A method according to claim 237, wherein the NDST1 IRC marker expression product is a NDST1 IRC transcript as set forth in any one of SEQ ID NO: 501 or 503.

239. A method according to claim 237, wherein the NDST1 IRC marker expression product is a NDST1 IRC polypeptide as set forth in any one of SEQ ID NO: 502 or 504.

240. A method according to claim 196 or claim 197, wherein the HIPK2 IRC marker expression product comprises a nucleotide sequence corresponding to an exon selected from HIPK2 exon 11 or an amino acid sequence encoded by that exon.

241. A method according to claim 240, wherein the HIPK2 IRC marker expression product is a HIPK2 IRC transcript as set forth in any one of SEQ ID NO: 505, 507, 509 or 511.

242. A method according to claim 240, wherein the HIPK2 IRC marker expression product is a HIPK2 IRC polypeptide as set forth in any one of SEQ ID NO: 506, 508, 510 or 512.

243. A method according to claim 196 or claim 197, wherein the CEACAM4 IRC marker expression product comprises a nucleotide sequence corresponding to an exon selected from CEACAM4 exon 5, 7, 23 or an amino acid sequence encoded by that exon.

244. A method according to claim 243, wherein the CEACAM4 IRC marker expression product is a CEACAM4 IRC transcript as set forth in any one of SEQ ID NO: 513 or 515.

245. A method according to claim 243, wherein the CEACAM4 IRC marker expression product is a CEACAM4 IRC polypeptide as set forth in any one of SEQ ID NO: 514 or 516.

246. A method according to any one of claims 1 to 245, comprising detecting the level of at least one IRC marker expression product from two or more of LISTS A, B, C, D, E and F.

247. A method according to claim 246, comprising detecting the level of at least one IRC marker expression product from LIST A and the level of at least one other IRC marker expression product from LIST B.

248. A method according to claim 246, comprising detecting the level of at least one IRC marker expression product from LIST A and the level of at least one other IRC marker expression product from LIST C.

249. A method according to claim 246, comprising detecting the level of at least one IRC marker expression product from LIST A and the level of at least one other IRC marker expression product from LIST D.

250. A method according to claim 246, comprising detecting the level of at least one IRC marker expression product from LIST A and the level of at least one other IRC marker expression product from LIST E.

251. A method according to claim 246, comprising detecting the level of at least one IRC marker expression product from LIST A and the level of at least one other IRC marker expression product from LIST F.

252. A method according to claim 246, comprising detecting the level of at least one IRC marker expression product from LIST B and the level of at least one other IRC marker expression product from LIST C.

253. A method according to claim 246, comprising detecting the level of at least one IRC marker expression product from LIST B and the level of at least one other IRC marker expression product from LIST D.

254. A method according to claim 246, comprising detecting the level of at least one IRC marker expression product from LIST B and the level of at least one other IRC marker expression product from LIST E.

255. A method according to claim 246, comprising detecting the level of at least one IRC marker expression product from LIST B and the level of at least one other IRC marker expression product from LIST F.

256. A method according to claim 246, comprising detecting the level of at least one IRC marker expression product from LIST C and the level of at least one other IRC marker expression product from LIST D.

257. A method according to claim 246, comprising detecting the level of at least one IRC marker expression product from LIST C and the level of at least one other IRC marker expression product from LIST E.

258. A method according to claim 246, comprising detecting the level of at least one IRC marker expression product from LIST C and the level of at least one other IRC marker expression product from LIST F.

259. A method according to claim 246, comprising detecting the level of at least one IRC marker expression product from LIST D and the level of at least one other IRC marker expression product from LIST E.

260. A method according to claim 246, comprising detecting the level of at least one IRC marker expression product from LIST D and the level of at least one other IRC marker expression product from LIST F.

261. A method according to claim 246, comprising detecting the level of at least one IRC marker expression product from LIST E and the level of at least one other IRC marker expression product from LIST F.

262. A method according to any one of claims 1 to 245, comprising detecting the level of at least one IRC marker expression product from each of three lists selected from LISTS A, B, C, D, E and F.

263. A method according to claim 262, comprising detecting the level of at least one IRC marker expression product from LIST A and the level of at least one other IRC marker expression product from LIST B and the level of at least one other IRC marker expression product from LIST C.

264. A method according to claim 262, comprising detecting the level of at least one IRC marker expression product from LIST A and the level of at least one other IRC marker expression product from LIST B and the level of at least one other IRC marker expression product from LIST D.

265. A method according to claim 262, comprising detecting the level of at least one IRC marker expression product from LIST A and the level of at least one other IRC marker expression product from LIST B and the level of at least one other IRC marker expression product from LIST E.

266. A method according to claim 262, comprising detecting the level of at least one IRC marker expression product from LIST A and the level of at least one other IRC marker expression product from LIST B and the level of at least one other IRC marker expression product from LIST F.

267. A method according to claim 262, comprising detecting the level of at least one IRC marker expression product from LIST B and the level of at least one other IRC marker expression product from LIST C and the level of at least one other IRC marker expression product from LIST D.

268. A method according to claim 262, comprising detecting the level of at least one IRC marker expression product from LIST B and the level of at least one other IRC marker expression product from LIST C and the level of at least one other IRC marker expression product from LIST E.

269. A method according to claim 262, comprising detecting the level of at least one IRC marker expression product from LIST B and the level of at least one other IRC marker expression product from LIST C and the level of at least one other IRC marker expression product from LIST F.

270. A method according to claim 262, comprising detecting the level of at least one IRC marker expression product from LIST C and the level of at least one other IRC marker expression product from LIST D and the level of at least one other IRC marker expression product from LIST E.

271. A method according to claim 262, comprising detecting the level of at least one IRC marker expression product from LIST C and the level of at least one other IRC marker expression product from LIST D and the level of at least one other IRC marker expression product from LIST F.

272. A method according to claim 262, comprising detecting the level of at least one IRC marker expression product from LIST D and the level of at least one other IRC marker expression product from LIST E and the level of at least one other IRC marker expression product from LIST F.

273. A method according to any one of claims 1 to 245, comprising detecting the level of at least one IRC marker expression product from each of four lists selected from LISTS A, B, C, D, E and F.

274. A method according to claim 273, comprising detecting the level of at least one IRC marker expression product from LIST A and the level of at least one other IRC marker expression product from LIST B and the level of at least one other IRC marker expression product from LIST C and the level of at least one other IRC marker expression product from LIST D.

275. A method according to claim 273, comprising detecting the level of at least one IRC marker expression product from LIST A and the level of at least one other IRC marker expression product from LIST B and the level of at least one other IRC marker expression product from LIST C and the level of at least one other IRC marker expression product from LIST E.

276. A method according to claim 273, comprising detecting the level of at least one IRC marker expression product from LIST A and the level of at least one other IRC marker expression product from LIST B and the level of at least one other IRC marker expression product from LIST C and the level of at least one other IRC marker expression product from LIST F.

277. A method according to claim 273, comprising detecting the level of at least one IRC marker expression product from LIST B and the level of at least one other IRC marker expression product from LIST C and the level of at least one other IRC marker expression product from LIST D and the level of at least one other IRC marker expression product from LIST E.

278. A method according to claim 273, comprising detecting the level of at least one IRC marker expression product from LIST B and the level of at least one other IRC marker expression product from LIST C and the level of at least one other IRC marker expression product from LIST D and the level of at least one other IRC marker expression product from LIST F.

279. A method according to claim 273, comprising detecting the level of at least one IRC marker expression product from LIST A and the level of at least one other IRC marker expression product from LIST C and the level of at least one other IRC marker expression product from LIST D and the level of at least one other IRC marker expression product from LIST E.

280. A method according to claim 273, comprising detecting the level of at least one IRC marker expression product from LIST A and the level of at least one other IRC marker expression product from LIST C and the level of at least one other IRC marker expression product from LIST D and the level of at least one other IRC marker expression product from LIST F.

281. A method according to claim 273, comprising detecting the level of at least one IRC marker expression product from LIST A and the level of at least one other IRC marker expression product from LIST D and the level of at least one other IRC marker expression product from LIST E and the level of at least one other IRC marker expression product from LIST F.

282. A method according to claim 273, comprising detecting the level of at least one IRC marker expression product from LIST A and the level of at least one other IRC marker expression product from LIST B and the level of at least one other IRC marker expression product from LIST D and the level of at least one other IRC marker expression product from LIST E.

283. A method according to claim 273, comprising detecting the level of at least one IRC marker expression product from LIST C and the level of at least one other IRC marker expression product from LIST D and the level of at least one other IRC marker expression product from LIST E and the level of at least one other RC marker expression product from LIST F.

284. A method according to any one of claims 1 to 245, comprising detecting the level of at least one IRC marker expression product from each of five lists selected from LISTS A, B, C, D, E and F.

285. A method according to claim 284, comprising detecting the level of at least one IRC marker expression product from LIST A and the level of at least one other IRC marker expression product from LIST B and the level of at least one other IRC marker expression product from LIST C and the level of at least one other IRC marker expression product from LIST D and the level of at least one other IRC marker expression product from LIST E.

286. A method according to claim 284, comprising detecting the level of at least one IRC marker expression product from LIST A and the level of at least one other IRC marker expression product from LIST B and the level of at least one other IRC marker expression product from LIST C and the level of at least one other IRC marker expression product from LIST D and the level of at least one other IRC marker expression product from LIST F.

287. A method according to claim 284, comprising detecting the level of at least one IRC marker expression product from LIST A and the level of at least one other IRC marker expression product from LIST C and the level of at least one other IRC marker expression product from LIST D and the level of at least one other IRC marker expression product from LIST E and the level of at least one other IRC marker expression product from LIST F.

288. A method according to claim 284, comprising detecting the level of at least one IRC marker expression product from LIST B and the level of at least one other IRC marker expression product from LIST C and the level of at least one other IRC marker expression product from LIST D and the level of at least one other IRC marker expression product from LIST E and the level of at least one other IRC marker expression product from LIST F.

289. A method according to claim 284, comprising detecting the level of at least one IRC marker expression product from LIST A and the level of at least one other IRC marker expression product from LIST B and the level of at least one other IRC marker expression product from LIST D and the level of at least one other IRC marker expression product from LIST E and the level of at least one other IRC marker expression product from LIST F.

290. A method according to any one of claims 1 to 245, comprising detecting the level of at least one IRC marker expression product from each of LISTS A, B, C, D, E and F.

291. A method according to claim 5, comprising diagnosing the absence of sepsis, inSIRS or post surgical inflammation when the measured level or functional activity of the or each IRC expression product is the same as or similar to the measured level or functional activity of the or each corresponding expression product when the control subject is a normal subject.

292. A method according to claim 291, wherein the measured level of an individual IRC expression product varies from the measured level of an individual corresponding expression product by no more than about 20%.

293. A method according to claim 5, wherein the biological sample comprises blood, especially peripheral blood, which suitably includes leukocytes.

294. A method for treating, preventing or inhibiting the development of at least one condition selected from sepsis, inSIRS or post-surgical inflammation in a subject, the method comprising:
comparing the level of at least one IRC expression product of a multi-transcript-producing gene in the subject to the level of a corresponding IRC marker expression product in at least one control subject selected from: a post-surgical inflammation-positive subject, an inSIRS positive subject, and a sepsis-positive subject, wherein a difference between the level of the at least one IRC marker expression product and the level of the corresponding IRC marker expression product indicates whether the subject has, or is at risk of developing, one of the conditions, wherein the at least one IRC marker expression product is predetermined as being differentially expressed between at least two of the conditions and wherein at least one other expression product from the multi-transcript producing gene is predetermined as being not so differentially expressed; and
administering to the subject, on the basis that the subject tests positive for sepsis, an effective amount of an agent that treats or ameliorates the symptoms or reverses or inhibits the development of sepsis, or
administering to the subject, on the basis that the subject tests positive for inSIRS, an effective amount of an agent that treats or ameliorates the symptoms or reverses or inhibits the development of inSIRS; or
administering to the subject, on the basis that the subject tests positive for post-surgical inflammation, an effective amount of an agent that treats or ameliorates the symptoms or reverses or inhibits the development of post-surgical inflammation.

295. A method according to claim 294, wherein the sepsis treatment or agent is selected from antibiotics, intravenous fluids, vasoactives, palliative support for damaged or distressed organs and close monitoring of vital organs.

296. A method according to claim 294, wherein the inSIRS treatment or agent is selected from antibiotics, steroids, intravenous fluids, glucocorticoids, vasoactives, palliative support for damaged or distressed organs (e.g. oxygen for respiratory distress, fluids for hypovolemia) and close monitoring of vital organs.

297. A method according to claim 294, wherein the post-surgical inflammation treatment or agent is selected from antibiotics, intravenous fluids and anti-inflammatory agents.
